(19) United States
(12) Patent Application Publication
(10) Pub. No.: US 2011/0244512 A1
(43) Pub. Date: Oct. 6, 2011

(54) PENTOSE PHOSPHATE PATHWAY UPREGULATION TO INCREASE PRODUCTION OF NON-NATIVE PRODUCTS OF INTEREST IN TRANSGENIC MICROORGANISMS

(75) Inventors: Seung-Pyo Hong, Hockessin, DE (US); Zhixiong Xue, Chadds Ford, PA (US); Quinn Qun Zhu, West Chester, PA (US)

(73) Assignee: E. I. DU PONT DE NEMOURS AND COMPANY, Wilmington, DE (US)

(21) Appl. No.: 13/074,069

(22) Filed: Mar. 29, 2011

Related U.S. Application Data
(60) Provisional application No. 61/319,473, filed on Mar. 31, 2010.

(51) Int. Cl. C12P 1/00; C12N 1/00; C12N 1/13; C12N 1/19; C12N 1/15

(52) U.S. Cl. 435/41; 435/243; 435/257.2; 435/254.2; 435/254.11

(57) ABSTRACT

Coordinately regulated over-expression of the genes encoding glucose 6-phosphate dehydrogenase [“G6PDH”] and 6-phospho-gluconolactonase [“6PGL”] in transgenic strains of the oleaginous yeast, Yarrowia lipolytica, comprising a functional polyunsaturated fatty acid [“PUFA”] biosynthetic pathway, resulted in increased production of PUFAs and increased total lipid content in the Yarrowia cells. This is achieved by increased cellular availability of the reduced form of nicotinamide adenine dinucleotide phosphate [“NADPH”], an important reducing equivalent for reductive biosynthetic reactions, within the transgenic microorganism.

This application claims the benefit of U.S. Provisional Application No. 61/319,473, filed Mar. 31, 2010, which is herein incorporated by reference in its entirety.

### FIELD OF THE INVENTION

This invention is in the field of biotechnology. More specifically, this invention pertains to methods useful for manipulating the cellular availability of the reduced form of nicotinamide adenine dinucleotide phosphate [“NADPH”] in transgenic microorganisms, based on coordinately regulated over-expression of pentose phosphate pathway genes (e.g., glucose-6-phosphate dehydrogenase [“G6PD”] and 6-phosphogluconolactonase [“6PGL”]).

### BACKGROUND OF THE INVENTION

The cofactor pair NADPH/NADP⁺ is essential for all living organisms, primarily as a result of its use as donor and/or acceptor of reducing equivalents in various oxidation-reduction reactions during anabolic metabolism. For example, NADPH is important for the production of amino acids, vitamins, aromatics, polyols, polyamines, hydroxyesters, isoprenoids, flavonoids and fatty acids including those that are polyunsaturated (e.g., omega-3 fatty acids and omega-6 fatty acids). In contrast, the cofactor pair NADH/NAD⁺ is used for catabolic activities within the cell.

A significant amount of NADPH reducing equivalents for reductive biosynthesis reactions within cells is produced via the pentose phosphate pathway [or “PP pathway”]. The PP pathway comprises a non-oxidative phase, responsible for the conversion of ribose-5-phosphate into substrates (i.e., glyceraldehyde-3-phosphate, fructose-6-phosphate) for the construction of nucleotides and nucleic acids, and an oxidative phase. The net reaction within the oxidative phase is set forth in the following chemical equation:

glucose 6-phosphate+2NADP⁺+H₂O→ribulose 5-phosphate+2NADPH+2H⁺+CO₂.

Production of many industrially useful compounds in recombinantly engineered organisms frequently increases cellular demand for NADPH. Optimization of the available NADPH thus is a useful means to maximize production of a compound(s) of interest. As such, several studies have demonstrated that increased quantities of NADPH in a recombinant organism results in increased quantities of the engineered product; however, numerous means have been utilized to achieve this goal.

One approach to increase cellular NADPH requires NADH. See, e.g., U.S. Pat. No. 5,830,716 which describes a method for production of increased L-threonine, L-lysine and L-phenylalanine in Escherichia coli, wherein the cells are modified by expression of a nicotinamide dinucleotide transhydrogenase (i.e., encoded by the E. coli pntA and pntB genes) so that increased NADPH is produced from NADH. Similarly, U.S. Pat. No. 7,326,557 describes a method of increasing the NADPH levels in E. coli by at least about 50%, by transformation of the host cell with a soluble pyridine nucleotide transhydrogenase (i.e., udhA), an enzyme that catalyzes the reversible reaction set forth as: NADH+NADP⁺ [symbol] NAD⁺+NADPH.

An alternate means to increase cellular NADPH is set forth in U.S. Pat. App. Pub. No. 2007-0087403 A1, which teaches strains of microorganisms having one or more of their NADPH-oxidizing activities limited and/or having one or more enzyme activities that allow the reduction of NADP⁺ favored. This can be accomplished by deletion of one or more genes coding for a quinine oxidoreductase or a soluble transhydrogenase. Additional optional modifications are also proposed, including deletion of a phosphoglucose isomerase or a phosphofructokinase and/or over-expression of glucose 6-phosphate dehydrogenase, 6-phosphogluconolactonase, 6-phosphogluconate dehydrogenase, isocitrate dehydrogenase, a membrane-bound transhydrogenase, 6-phosphogluconate dehydratase, malate synthase, isocitrate lyase, or isocitrate dehydrogenase kinase/phosphatase.

Previous methods have not manipulated genes directly within the oxidative phase of the PP pathway, which is responsible for production of NADPH from NADP⁺, in conjunction with the reduction of glucose-6-phosphate [“G-6-P”] to ribulose 5-phosphate. The oxidative branch of the PP pathway includes three consecutive reactions, as described below in Table 1 and FIG. 1.

TABLE 1
Reactions In The Oxidative Phase Of The Pentose Phosphate
Pathway

| Reactants | Products | Enzyme | Description |
|---|---|---|---|
| Glucose 6- | delta-6-phospho- | glucose 6- | Dehydrogenation. |
| phosphate + | gluconolactone + | phosphate | The hemiacetal |
| NADP⁺ | NADPH | dehydrogenase | hydroxyl group |
| | | [“G6PDH”] | located on carbon |
| | | E.C. 1.1.1.49 | 1 of glucose |
| | | | 6-phosphate is |
| | | | converted into a |
| | | | carbonyl group, |
| | | | generating a |
| | | | lactone, and, |
| | | | in the process, |
| | | | NADPH is |
| | | | generated. |
| delta-6- | 6-phospho- | 6-phospho- | Hydrolysis. |
| phospho- | gluconate + H⁺ | glucono- | |
| gluconolactone + | | lactonase | |
| H₂O | | [“6PGL”] | |
| | | E.C. 3.1.1.31 | |
| 6-phospho- | ribulose 5- | 6-phospho- | Oxidative |
| gluconate + | phosphate + | gluconate | decarboxylation. |
| NADP⁺ | NADPH + CO₂ | dehydrogenase | NADP⁺ is the |
| | | [6PGDH”] | electron acceptor, |
| | | E.C. 1.1.1.44 | generating |
| | | | another |
| | | | molecule of |
| | | | NADPH, a CO₂, |
| | | | and ribulose |
| | | | 5-phosphate. |

While it may be obvious to try and over-express glucose 6-phosphate dehydrogenase [“G6PDH”] as a means to increase production of NADPH, it is also lethal. Specifically, the product of this enzymatic reaction, i.e., delta-6-phosphogluconolactone, can be toxic to the cell. For example, Hager, P. W. et al. (J. Bacteriology, 182(14):3934-3941 (2000)) describe creation of a mutant strain of Pseudomonas aeruginosa in which the devB/SOL homolog encoding 6PGL was inactivitated. This mutant grew at only 9% of the wildtype rate using mannitol as the carbon source and at 50% of the wildtype rate using gluconate as the carbon source, thereby leading to the hypothesis that increased concentrations of 6-phosphogluconate were toxic to the cell. It is stated that “It seems essential that there should be similar amounts of 6PGL and G6PDH activity in the cell in order to maintain a balanced flux through this metabolic pathway.” Several organisms have 6PGL and G6PDH homologs that overlap on the chromosome on which they are co-located, further suggesting a very tight transcriptional control and the possibility of coordinately regulated expression. One solution to the need for efficient metabolic flux through 6PGL and G6PDH appears to be found in those animals having both enzymatic activities combined within a single protein.

Further insight into 6PGL and G6PDH regulation was gained following the NMR spectroscopic analysis of Miclet, E. et al. (J. Biol. Chem., 276(37):34840-34846 (2001)). This study showed that the delta form of 6-phosphogluconolactone [“δ-6-P-G-L”] was the only product of G-6-P oxidation, with the gamma form of 6-phosphogluconolactone [“γ-6-P-G-L”] produced subsequently by intermolecular rearrangement; however, only δ-6-P-G-L can be hydrolysed by 6PGL, while γ-6-P-G-L is a “dead end” that is unable to undergo further conversion. On the basis of this observation, Miclet et al. concluded that 6PGL activity accelerates hydrolysis of the delta form, thus preventing its conversion into the gamma form and 6PGL guards against the accumulation of δ-6-P-G-L, which may be toxic through its reaction with endogenous cellular nucleophiles and interrupt the functioning of the PP pathway.

Despite the difficulties noted above with respect to over-expression of G6PDH, Aon, J. C. et al. (AEM, 74(4):950-958 (2008)) report successful over-expression of 6PGL in Escherichia coli as a means to suppress the formation of gluconoylated adducts in heterologously expressed proteins. Specifically, a Pseudomonas aeruginosa gene encoding 6PGL expressed in E. coli BL21(DE3) cells was found to increase the biomass yield and specific productivity of a heterologous 18-kDa protein by 50% and 60%, respectively. It was concluded that the higher level of 6PGL expression allowed the strain to satisfy the extra demand for precursors, as well as the energy requirements, in order to replicate plasmid DNA and express heterologous genes, as metabolic flux analysis showed by the higher precursor and NADPH fluxes through the oxidative branch of the PP pathway.

Similarly, Ren, L.-J. et al. (Bioprocess Biosyst. Eng., 32:837-843 (2009)) appreciated the significance of ensuring an appropriate supply of NADPH during the biosynthesis of the omega-3 polyunsaturated fatty acid, docosahexaenoic acid [“DHA”], in Schizochytrium sp. HX-308. However, the solution utilized therein involved addition of malic acid to the fermentation system during the rapid lipid accumulation phase of the fermentation process, to enable conversion of malate to pyruvate with simultaneous reduction of NADP⁺ to NADPH. This modification prevented a deficiency in cellular NADPH and permitted a 15% increase in the total lipids accumulated in the organism and an increase from 35% to 60% in the final DHA content of total fatty acids.

Disclosed herein is a means to over-express both glucose-6-phosphate dehydrogenase [“G6PD”] and 6-phosphogluconolactonase [“6PGL”] as a means to enable increased cellular availability of the cofactor NADPH in transgenic microorganisms recombinantly engineered to produce a heterologous non-native product of interest. Optimization of cellular NADPH will result in increased production of heterologous products of interest, when these products of interest require the NADPH cofactor for their biosynthesis.

### SUMMARY

In a first embodiment, the invention concerns a transgenic microorganism comprising:
(a) at least one gene encoding glucose-6-phosphate dehydrogenase;
(b) at least one gene encoding 6-phosphogluconolactonase; and,
(c) at least one heterologous gene encoding a non-native product of interest;

wherein biosynthesis of the non-native product of interest comprises at least one enzymatic reaction that requires nicotinamide adenine dinucleotide phosphate;

wherein coordinately regulated over-expression of (a) and (b) results in an increased quantity of nicotinamide adenine dinucleotide phosphate; and,

wherein the increased quantity of nicotinamide adenine dinucleotide phosphate results in an increased quantity of the product of interest produced by expression of (c) in the transgenic microorganism when compared to the quantity of nicotinamide adenine dinucleotide phosphate and the quantity of the product of interest produced by a transgenic microorganism comprising (c) and either lacking or not over-expressing (a) and (b) in a coordinately regulated fashion.

Furthermore, the coordinately regulated over-expression of the at least one gene encoding G6PDH and the at least one gene encoding 6PGL is achieved by a means selected from the group consisting of:
(a) the at least one gene encoding G6PDH is operably linked to a first promoter and the at least one gene encoding 6PGL is operably linked to a second promoter, wherein the first promoter has equivalent or reduced activity when compared to the second promoter;
(b) the at least one gene encoding G6PDH is expressed in multicopy and the at least one gene encoding 6PGL is expressed in multicopy, wherein the copy number of the at least one gene encoding G6PDH is equivalent or reduced when compared to the copy number of the at least one gene encoding 6PGL;
(c) the enzymatic activity of the at least one gene encoding G6PDH is linked to the enzymatic activity of the at least one gene encoding 6PGL as a multizyme; and,
(d) a combination of any of the means set forth in (a), (b) and (c).

In a second embodiment, the invention concerns the transgenic microorganism supra wherein at least one gene encoding 6-phosphogluconate dehydrogenase is expressed in addition to the genes of (a), (b) and (c).

In a third embodiment, the invention concerns the transgenic microorganism supra, wherein the non-native product of interest is selected from the group consisting of: polyunsaturated fatty acids, carotenoids, amino acids, vitamins, sterols, flavonoids, organic acids, polyols and hydroxyesters.

In a fourth embodiment, the invention concerns the transgenic microorganism supra wherein:
(a) the non-native product of interest is selected from the group consisting of: an omega-3 fatty acid and an omega-6 fatty acid; and,
(b) the at least one heterologous gene of (c) is selected from the group consisting of: delta-12 desaturase, delta-6 desaturase, delta-8 desaturase, delta-5 desaturase, delta-17 desaturase, delta-15 desaturase, delta-9 desaturase, delta-4 desaturase, C_{14/16} elongase, C_{16/18} elongase, C_{18/20} elongase, C_{20/22} elongase and delta-9 elongase.

In a fifth embodiment, the invention concerns the transgenic microorganism wherein said transgenic microorganism is selected from the group consisting of: algae, yeast, euglenoids, stramenopiles, oomycetes and fungi. More particularly, the preferred transgenic microorganism is an oleaginous yeast.

In a sixth embodiment, the invention concerns a transgenic oleaginous yeast comprising:
(a) at least one gene encoding glucose-6-phosphate dehydrogenase;
(b) at least one gene encoding 6-phosphogluconolactonase; and,
(c) at least one heterologous gene encoding a non-native product of interest, wherein the product of interest is selected from the group consisting of: at least one polyunsaturated fatty acid, at least one quinone-derived compound, at least one carotenoid and at least one sterol;

wherein coordinately regulated over-expression of (a) and (b) results in an increased quantity of nicotinamide adenine dinucleotide phosphate;

and,

wherein the increased quantity of nicotinamide adenine dinucleotide phosphate results in an increased quantity of the product of interest produced by expression of (c) in the transgenic oleaginous yeast when compared to the quantity of nicotinamide adenine dinucleotide phosphate and the quantity of the product of interest produced by a transgenic oleaginous yeast comprising (c) and either lacking or not over-expressing (a) and (b) in a coordinately regulated fashion.

More particularly, the transgenic oleaginous yeast of the invention is Yarrowia lipolytica.

In a seventh embodiment, the invention concerns the transgenic oleaginous yeast supra wherein the at least one polyunsaturated fatty acid is selected from the group consisting of: linoleic acid, gamma-linolenic acid, eicosadienoic acid, dihomo-gamma-linolenic acid, arachidonic acid, docosatetraenoic acid, omega-6 docosapentaenoic acid, alpha-linolenic acid, stearidonic acid, eicosatrienoic acid, eicosatetraenoic acid, eicosapentaenoic acid, omega-3 docosapentaenoic acid and docosahexaenoic acid.

In an eighth embodiment, the invention concerns the transgenic oleaginous yeast supra wherein the total lipid content is increased in addition to the quantity of nicotinamide adenine dinucleotide phosphate and the quantity of the at least one polyunsaturated fatty acid, when compared to the total lipid content produced by a transgenic oleaginous yeast comprising (c) and either lacking or not over-expressing (a) and (b) in a coordinately regulated fashion.

In a ninth embodiment, the invention concerns the transgenic oleaginous yeast supra wherein the at least one carotenoid is selected from the group consisting of: antheraxanthin, adonirubin, adonixanthin, astaxanthin, canthaxanthin, capsorubrin, β-cryptoxanthin, α-carotene, β-carotene, β, ψ-carotene, δ-carotene, ε-carotene, echinenone, 3-hydroxyechinenone, 3′-hydroxyechinenone, γ-carotene, ψ-carotene, 4-keto-γ-carotene, ζ-carotene, α-cryptoxanthin, deoxyflexixanthin, diatoxanthin, 7,8-didehydroastaxanthin, didehydrolycopene, fucoxanthin, fucoxanthinol, isorenieratene, β-isorenieratene, lactucaxanthin, lutein, lycopene, myxobactone, neoxanthin, neurosporene, hydroxyneurosporene, peridinin, phytoene, phytofluene, rhodopin, rhodopin glucoside, 4-keto-rubixanthin, siphonaxanthin, spheroidene, spheroidenone, spirilloxanthin, torulene, 4-keto-torulene, 3-hydroxy-4-keto-torulene, uriolide, uriolide acetate, violaxanthin, zeaxanthin-β-diglucoside, zeaxanthin, a C₃₀ carotenoid, and combinations thereof.

In a tenth embodiment, the invention concerns the transgenic oleaginous yeast supra wherein the at least one quinone-derived compound is selected from the group consisting of: a ubiquinone, a vitamin K compound, and a vitamin E compound, and combinations thereof.

In an eleventh embodiment, the invention concerns the transgenic oleaginous yeast supra wherein the at least one sterol compound is selected from the group consisting of: squalene, lanosterol, zymosterol, ergosterol, 7-dehydrocholesterol (provitamin D3), and combinations thereof.

In a twelfth embodiment, the invention concerns a method for the production of a non-native product of interest comprising:
(a) providing a transgenic microorganism comprising:
   (i) at least one gene encoding glucose-6-phosphate dehydrogenase;
   (ii) at least one gene encoding 6-phosphogluconolactonase; and,
   (iii) at least one heterologous gene encoding a non-native product of interest;
      wherein (i) and (ii) are over-expressed in a coordinately regulated fashion and wherein an increased quantity of nicotinamide adenine dinucleotide phosphate is produced when compared to the quantity of nicotinamide adenine dinucleotide phosphate produced by a transgenic microorganism either lacking or not over-expressing (i) and (ii) in a coordinately regulated fashion;
(b) growing the transgenic microorganism of step (a) in the presence of a fermentable carbon source whereby expression of (iii) results in production of the non-native product of interest; and,
(c) optionally recovering the non-native product of interest.

### Biological Deposits

The following biological material has been deposited with the American Type Culture Collection (ATCC), 10801 University Boulevard, Manassas, Va. 20110-2209, and bears the following designation, accession number and date of deposit.

| Biological Material | Accession No. | Date of Deposit |
|---|---|---|
| Yarrowia lipolytica Y4128 | ATCC PTA-8614 | Aug. 23, 2007 |

The biological material listed above was deposited under the terms of the Budapest Treaty on the International Recognition of the Deposit of Microorganisms for the Purposes of Patent Procedure. The listed deposit will be maintained in the indicated international depository for at least 30 years and will be made available to the public upon the grant of a patent disclosing it. The availability of a deposit does not constitute a license to practice the subject invention in derogation of patent rights granted by government action.

Yarrowia lipolytica Y4305U was derived from Yarrowia lipolytica Y4128, according to the methodology described in U.S. Pat. App. Pub. No. 2008-0254191.

### BRIEF DESCRIPTION OF THE DRAWINGS AND SEQUENCE LISTINGS

FIG. 1 diagrams the biochemical reactions that occur during the oxidative phase of the pentose phosphate pathway.

FIG. 2 provides plasmid maps for the following: (A) pZWF-MOD1; and, (B) pZUF-MOD1.

FIG. 3 provides plasmid maps for the following: (A) pZKLY-PP2; and, (B) pZKLY-6PGL.

FIG. 4 provides a plasmid map for the following: (A) pGPM-G6PD.

The invention can be more fully understood from the following detailed description and the accompanying sequence descriptions, which form a part of this application.

The following sequences comply with 37 C.F.R. §1.821-1.825 (“Requirements for Patent Applications Containing Nucleotide Sequences and/or Amino Acid Sequence Disclosures—the Sequence Rules”) and are consistent with World Intellectual Property Organization (WIPO) Standard ST. 25 (1998) and the sequence listing requirements of the EPO and PCT (Rules 5.2 and 49.5 (a-bis), and Section 208 and Annex C of the Administrative Instructions). The symbols and format used for nucleotide and amino acid sequence data comply with the rules set forth in 37 C.F.R. §1.822.

SEQ ID NOs:1-25 are ORFs encoding genes or proteins (or portions thereof), or plasmids, as identified in Table 2.

TABLE 2
Summary Of Nucleic Acid And Protein SEQ ID Numbers

| | | Protein |
|---|---|---|
| | Nucleic acid | SEQ |
| Description and Abbreviation | SEQ ID NO. | ID NO. |
| Yarrowia lipolytica YALI0E22649p | 1 | 2 |
| (Gen Bank Accession No. XM_504275) | (1497 bp) | (498 AA) |
| [“G6PDH”] | | |
| Yarrowia lipolytica YALI0E11671p | 3 | 4 |
| (Gen Bank Accession No. XM_503830) | (747 bp) | (248 AA) |
| [“6PGL”] | | |
| Yarrowia lipolytica YALI0B15598p | 5 | 6 |
| (GenBank Accession No. XM_500938) | (1470 bp) | (489 AA) |
| [“6PGDH”] | | |
| Plasmid pZWF-MOD1 | 7 | — |
| | (9028 bp) | |
| Primer YZWF-F1 | 8 | — |
| Primer YZWF-R | 9 | — |
| Genomic DNA encoding Yarrowia lipolytica | 10 | 11 |
| G6PDH | (1937 bp) | (498 AA) |
| G6PDH intron | 12 | — |
| | (440 bp) | |
| Plasmid pZUF-MOD1 | 13 | — |
| | (7323 bp) | |
| Yarrowia lipolytica fructose-bisphosphate | 14 | — |
| aldolase + intron promoter [“FBAIN”] | (973 bp) | |
| Plasmid pZKLY-PP2 | 15 | — |
| | (11,180 bp) | |
| Primer YL961 | 16 | — |
| Primer YL962 | 17 | — |
| Yarrowia lipolytica fructose-bisphosphate | 18 | — |
| aldolase promoter [“FBA”] | (1001 bp) | |
| Plasmid pZKLY-6PGL | 19 | — |
| | (8585 bp) | |
| Primer YL959 | 20 | — |
| Primer YL960 | 21 | — |
| Plasmid pDMW224-S2 | 22 | — |
| | (9519 bp) | |
| Plasmid pGPM-G6PD | 23 | — |
| | (8500 bp) | |
| Yarrowia lipolytica phosphoglycerate mutase | 24 | — |
| promoter [“GPM”] | (878 bp) | |
| Plasmid pZKLY | 25 | — |
| | (9045 bp) | |

### DETAILED DESCRIPTION OF THE INVENTION

The disclosures of all patent and non-patent literature cited herein are incorporated by reference in their entirety.

In this disclosure, the following abbreviations are used:

“Open reading frame” is abbreviated as “ORF”.

“Polymerase chain reaction” is abbreviated as “PCR”.

“American Type Culture Collection” is abbreviated as “ATCC”.

“Pentose phosphate pathway” is abbreviated as “PP pathway”.

“Nicotinamide adenine dinucleotide phosphate” is abbreviated as “NADP⁺” or, in its reduced form, “NADPH”.

“Glucose 6-phosphate” is abbreviated as “G-6-P”.

“Glucose-6-phosphate dehydrogenase” is abbreviated as “G6PDH”.

“6-phosphogluconolactonase” is abbreviated as “6PGL”.

“6-phosphogluconate dehydrogenase” is abbreviated as “6PGDH”

“Polyunsaturated fatty acid(s)” is abbreviated as “PUFA(s)”.

“Triacylglycerols” are abbreviated as “TAGs”.

“Total fatty acids” are abbreviated as “TFAs”.

“Fatty acid methyl esters” are abbreviated as “FAMEs”.

“Dry cell weight” is abbreviated as “DCW”.

As used herein, the term “invention” or “present invention” is not meant to be limiting but applies generally to any of the inventions defined in the claims or described herein.

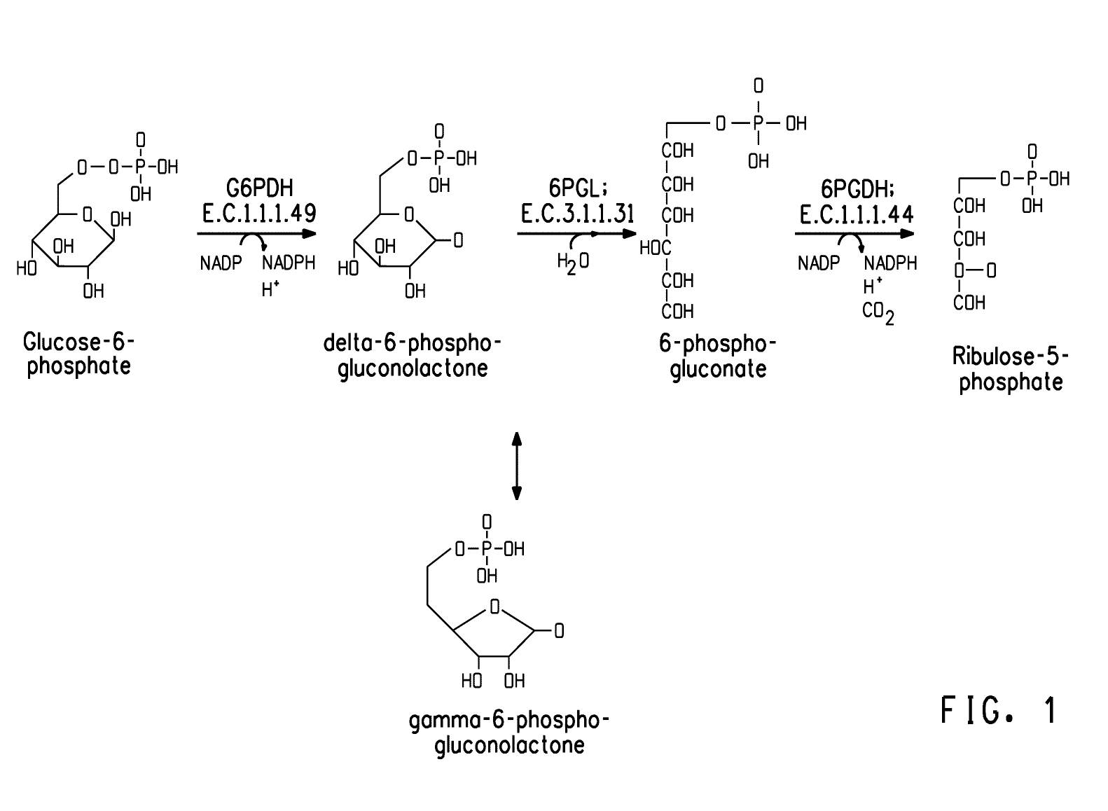

The term “pentose phosphate pathway” [“PP pathway”], “phosphogluconate pathway” and “hexose monophosphate shunt pathway” refers to a cytosolic process that occurs in two distinct phases. The non-oxidative phase is responsible for conversion of ribose-5-phosphate into substrates for the construction of nucleotides and nucleic acids. The oxidative phase, which can be summarized in the following chemical reaction: glucose 6-phosphate+2 NADP⁺+H₂O→ribulose 5-phosphate+2 NADPH+2H⁺+CO₂, serves to generate NADPH reducing equivalents for reductive biosynthesis reactions within cells. More specifically, the reactions that occur in the oxidative phase comprise a dehydrogenation, hydrolysis and an oxidative decarboxylation, as previously described in Table 1 and FIG. 1.

“Nicotinamide adenine dinucleotide phosphate” [“NADP⁺”], and its reduced form NADPH, are a cofactor pair having CAS Registry No. 53-59-8. NADP⁺ is used in anabolic reactions which require NADPH as a reducing agent. In animals, the oxidative phase of the PP pathway is the major source of NADPH in cells, producing approximately 60% of the NADPH required. NADPH provides reducing equivalents for cytochrome P450 hydroxylation (e.g., of aromatic compounds, steroids, alcohols) and various biosynthetic reactions (e.g., fatty acid chain elongation and lipid, cholesterol and isoprenoid synthesis). Additionally, NADPH provides reducing equivalents for oxidation-reduction involved in protection against the toxicity of reactive oxygen species.

The term “glucose-6-phosphate dehydrogenase” [“G6PD”] refers to an enzyme that catalyzes the conversion of glucose-6-phosphate [“G-6-P”] to a 6-phosphogluconolactone via dehydrogenation [E.C. 1.1.1.49].

The term “6-phosphogluconolactone” refers to compounds having CAS Registry No. 2641-81-8. These phosphogluconolactones are in either a delta-form or gamma-form through intramolecular conversion.

The term “6-phosphogluconolactonase” [“6PGL”] refers to an enzyme that catalyzes the conversion of delta-6-phospho-gluconolactone to 6-phospho-gluconate by hydrolysis [E.C. 3.1.1.31].

The term “6-phosphogluconate” refers to compounds having CAS Registry No. 921-62-0.

The term “6-phosphogluconate dehydrogenase” [“6PGDH”] refers to an enzyme that catalyzes the conversion of 6-phosphogluconate to ribulose-5-phosphate, along with NADPH and carbon dioxide via oxidative decarboxylation [E.C. 1.1.1.44].

The term “coordinately regulated over-expression of G6PD and 6PGL” means that approximately similar amounts of G6PDH and 6PGL activity are co-expressed in the cell in order to maintain a balanced flux through the PP pathway, or such that the G6PDH activity is less than the 6PGL activity. This ensures that the 6PGL activity accelerates hydrolysis of the delta form of 6-phosphogluconolactone [“δ-6-P-G-L”], thus preventing its conversion into the gamma form [“γ-6-P-G-L”], and prevents accumulation of significant concentrations of δ-6-P-G-L.

The term “expressed in multicopy” means that the gene copy number is greater than one.

The term “multizyme” or “fusion protein” refers to a single polypeptide having at least two independent and separable enzymatic activities, wherein the first enzymatic activity is preferably linked to the second enzymatic activity (U.S. Pat. Appl. Pub. No. 2008-0254191-A1). The “link” or “bond” between the at least two independent and separable enzymatic activities is minimally comprised of a single polypeptide bond, although the link may also be comprised of one amino acid residue, such as proline or glycine, or a polypeptide comprising at least one proline or glycine amino acid residue. U.S. Pat. Appl. Pub. No. 2008-0254191-A1 also describes some preferred linkers, selected from the group consisting of: SEQ ID NO:1, SEQ ID NO:2, SEQ ID NO:3, SEQ ID NO:4, SEQ ID NO:5, SEQ ID NO:6 and SEQ ID NO:7 therein.

The term “non-native product of interest” refers to any product that is not naturally produced in a wildtype microorganism. Typically, the non-native product of interest is produced via recombinant means, such that the appropriate heterologous gene(s) is introduced into the host microorganism to enable expression of the heterologous protein, which is the product of interest. For the purposes of the present invention herein, biosynthesis of a non-native product of interest requires at least one enzymatic reaction that utilizes NADPH as a reducing equivalent. Non-limiting examples of preferred non-native products of interest include, but are not limited to, polyunsaturated fatty acids, carotenoids, amino acids, vitamins, sterols, flavonoids, organic acids, polyols and hydroxyesters.

The term “at least one heterologous gene encoding a non-native product of interest” refers to a gene(s) derived from a different origin than of the host microorganism into which it is introduced. The heterologous gene facilitates production of a non-native product of interest in the host microorganism. In some cases, only a single heterologous gene may be needed to enable production of the product of interest, catalyzing conversion of a substrate directly into the desired product of interest without any intermediate steps or pathway intermediates. Alternatively, it may be desirable to introduce a series of genes encoding a novel biosynthetic pathway into the microorganism, such that a series of reactions occur to produce a desired non-native product of interest.

The term “oleaginous” refers to those organisms that tend to store their energy source in the form of oil (Weete, In: Fungal Lipid Biochemistry, 2^{nd} Ed., Plenum, 1980). Generally, the cellular oil content of oleaginous microorganisms follows a sigmoid curve, wherein the concentration of lipid increases until it reaches a maximum at the late logarithmic or early stationary growth phase and then gradually decreases during the late stationary and death phases (Yongmanitchai and Ward, Appl. Environ. Microbiol., 57:419-25 (1991)). It is not uncommon for oleaginous microorganisms to accumulate in excess of about 25% of their dry cell weight as oil.

The term “oleaginous yeast” refers to those microorganisms classified as yeasts that can make oil. Examples of oleaginous yeast include, but are no means limited to, the following genera: Yarrowia, Candida, Rhodotorula, Rhodosporidium, Cryptococcus, Trichosporon and Lipomyces.

The terms “polynucleotide”, “polynucleotide sequence”, “nucleic acid sequence”, “nucleic acid fragment” and “isolated nucleic acid fragment” are used interchangeably herein. These terms encompass nucleotide sequences and the like. A polynucleotide may be a polymer of RNA or DNA that is single- or double-stranded, that optionally contains synthetic, non-natural or altered nucleotide bases. A polynucleotide in the form of a polymer of DNA may be comprised of one or more segments of cDNA, genomic DNA, synthetic DNA, or mixtures thereof. Nucleotides (usually found in their 5′-monophosphate form) are referred to by a single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

A nucleic acid fragment is “hybridizable” to another nucleic acid fragment, such as a cDNA, genomic DNA, or RNA molecule, when a single-stranded form of the nucleic acid fragment can anneal to the other nucleic acid fragment under the appropriate conditions of temperature and solution ionic strength. Hybridization and washing conditions are well known and exemplified in Sambrook, J., Fritsch, E. F. and Maniatis, T. Molecular Cloning: A Laboratory Manual, 2^{nd} ed., Cold Spring Harbor Laboratory: Cold Spring Harbor, N.Y. (1989), which is hereby incorporated herein by reference, particularly Chapter 11 and Table 11.1. The conditions of temperature and ionic strength determine the “stringency” of the hybridization. Stringency conditions can be adjusted to screen for moderately similar fragments (such as homologous sequences from distantly related organisms), to highly similar fragments (such as genes that duplicate functional enzymes from closely related organisms). Post-hybridization washes determine stringency conditions. One set of preferred conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. A more preferred set of stringent conditions uses higher temperatures in which the washes are identical to those above except for the temperature of the final two 30 min washes in 0.2×SSC, 0.5% SDS was increased to 60° C. Another preferred set of highly stringent conditions uses two final washes in 0.1×SSC, 0.1% SDS at 65° C. An additional set of stringent conditions include hybridization at 0.1×SSC, 0.1% SDS, 65° C. and washes with 2×SSC, 0.1% SDS followed by 0.1×SSC, 0.1% SDS, for example.

Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of the thermal melting point [“Tₘ” or “Tm”] for hybrids of nucleic acids having those sequences. The relative stability, corresponding to higher Tm, of nucleic acid hybridizations decreases in the following order: RNA:RNA, DNA:RNA, DNA:DNA. For hybrids of greater than 100 nucleotides in length, equations for calculating Tm have been derived (see Sambrook et al., supra, 9.50-9.51). For hybridizations with shorter nucleic acids, i.e., oligonucleotides, the position of mismatches becomes more important, and the length of the oligonucleotide determines its specificity (see Sambrook et al., supra, 11.7-11.8). In one embodiment the length for a hybridizable nucleic acid is at least about 10 nucleotides. Preferably a minimum length for a hybridizable nucleic acid is at least about 15 nucleotides; more preferably at least about 20 nucleotides; and most preferably the length is at least about 30 nucleotides. Furthermore, the skilled artisan will recognize that the temperature and wash solution salt concentration may be adjusted as necessary according to factors such as length of the probe.

A “substantial portion” of an amino acid or nucleotide sequence is that portion comprising enough of the amino acid sequence of a polypeptide or the nucleotide sequence of a gene to putatively identify that polypeptide or gene, either by manual evaluation of the sequence by one skilled in the art, or by computer-automated sequence comparison and identification using algorithms such as the Basic Local Alignment Search Tool [“BLAST”] (Altschul, S. F., et al., J. Mol. Biol., 215:403-410 (1993)). In general, a sequence of ten or more contiguous amino acids or thirty or more nucleotides is necessary in order to putatively identify a polypeptide or nucleic acid sequence as homologous to a known protein or gene. Moreover, with respect to nucleotide sequences, gene specific oligonucleotide probes comprising 20-30 contiguous nucleotides may be used in sequence-dependent methods of gene identification (e.g., Southern hybridization) and isolation, such as, in situ hybridization of microbial colonies or bacteriophage plaques. In addition, short oligonucleotides of 12-15 bases may be used as amplification primers in PCR in order to obtain a particular nucleic acid fragment comprising the primers. Accordingly, a “substantial portion” of a nucleotide sequence comprises enough of the sequence to specifically identify and/or isolate a nucleic acid fragment comprising the sequence. The skilled artisan, having the benefit of the sequences as reported herein, may now use all or a substantial portion of the disclosed sequences for purposes known to those skilled in this art, based on the methodologies described herein.

The term “complementary” is used to describe the relationship between nucleotide bases that are capable of hybridizing to one another. For example, with respect to DNA, adenosine is complementary to thymine and cytosine is complementary to guanine.

The terms “homology” and “homologous” are used interchangeably. They refer to nucleic acid fragments wherein changes in one or more nucleotide bases do not affect the ability of the nucleic acid fragment to mediate gene expression or produce a certain phenotype. These terms also refer to modifications of the nucleic acid fragments such as deletion or insertion of one or more nucleotides that do not substantially alter the functional properties of the resulting nucleic acid fragment relative to the initial, unmodified fragment.

Moreover, the skilled artisan recognizes that homologous nucleic acid sequences are also defined by their ability to hybridize, under moderately stringent conditions, such as 0.5×SSC, 0.1% SDS, 60° C., with the sequences exemplified herein, or to any portion of the nucleotide sequences disclosed herein and which are functionally equivalent thereto. Stringency conditions can be adjusted to screen for moderately similar fragments.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have at least about 80% sequence identity, or 90% sequence identity, up to and including 100% sequence identity (i.e., fully complementary) with each other.

The term “stringent conditions” or “stringent hybridization conditions” includes reference to conditions under which a probe will selectively hybridize to its target sequence. Stringent conditions are sequence-dependent and will be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing). Generally, a probe is less than about 1000 nucleotides in length, optionally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C.

Specificity is typically the function of post-hybridization washes, the important factors being the ionic strength and temperature of the final wash solution. For DNA-DNA hybrids, the Tₘ can be approximated from the equation of Meinkoth et al., Anal. Biochem., 138:267-284 (1984): Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with ≧90% identity are sought, the T, can be decreased 10° C. Generally, stringent conditions are selected to be about 5° C. lower than the Tₘ for the specific sequence and its complement at a defined ionic strength and pH. However, severely stringent conditions can utilize a hybridization and/or wash at 1, 2, 3, or 4° C. lower than the Tₘ; moderately stringent conditions can utilize a hybridization and/or wash at 6, 7, 8, 9, or 10° C. lower than the Tₘ; and, low stringency conditions can utilize a hybridization and/or wash at 11, 12, 13, 14, 15, or 20° C. lower than the Tₘ. Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Tijssen, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, New York (1993); and Current Protocols in Molecular Biology, Chapter 2, Ausubel et al., Eds., Greene Publishing and Wiley-Interscience, New York (1995). Hybridization and/or wash conditions can be applied for at least 10, 30, 60, 90, 120 or 240 minutes.

The term “percent identity” refers to a relationship between two or more polypeptide sequences or two or more polynucleotide sequences, as determined by comparing the sequences. “Percent identity” also means the degree of sequence relatedness between polypeptide or polynucleotide sequences, as the case may be, as determined by the percentage of match between compared sequences. “Percent identity” and “percent similarity” can be readily calculated by known methods, including but not limited to those described in: 1) Computational Molecular Biology (Lesk, A. M., Ed.) Oxford University: NY (1988); 2) Biocomputing: Informatics and Genome Protects (Smith, D. W., Ed.) Academic: NY (1993); 3) Computer Analysis of Sequence Data, Part I (Griffin, A. M., and Griffin, H. G., Eds.) Humania: NJ (1994); 4) Sequence Analysis in Molecular Biology (von Heinje, G., Ed.) Academic (1987); and, 5) Sequence Analysis Primer (Gribskov, M. and Devereux, J., Eds.) Stockton: NY (1991).

Preferred methods to determine percent identity are designed to give the best match between the sequences tested. Methods to determine percent identity and percent similarity are codified in publicly available computer programs. Sequence alignments and percent identity calculations may be performed using the MegAlign™ program of the LASERGENE bioinformatics computing suite (DNASTAR Inc., Madison, Wis.). Multiple alignment of the sequences is performed using the “Clustal method of alignment” which encompasses several varieties of the algorithm including the “Clustal V method of alignment” and the “Clustal W method of alignment” (described by Higgins and Sharp, CABIOS, 5:151-153 (1989); Higgins, D. G. et al., Comput. Appl. Biosci., 8:189-191 (1992)) and found in the MegAlign™ (version 8.0.2) program of the LASERGENE bioinformatics computing suite (DNASTAR Inc.). After alignment of the sequences using either Clustal program, it is possible to obtain a “percent identity” by viewing the “sequence distances” table in the program.

The “BLASTN method of alignment” is an algorithm provided by the National Center for Biotechnology Information [“NCBI”] to compare nucleotide sequences using default parameters, while the “BLASTP method of alignment” is an algorithm provided by the NCBI to compare protein sequences using default parameters.

It is well understood by one skilled in the art that many levels of sequence identity are useful in identifying polypeptides, from other species, wherein such polypeptides have the same or similar function or activity. Suitable nucleic acid fragments, i.e., isolated polynucleotides encoding polypeptides in the methods and host cells described herein, encode polypeptides that are at least about 70-85% identical, while more preferred nucleic acid fragments encode amino acid sequences that are at least about 85-95% identical to the amino acid sequences reported herein. Although preferred ranges are described above, useful examples of percent identities include any integer percentage from 50% to 100%, such as 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%. Also, of interest is any full-length or partial complement of this isolated nucleotide fragment.

Suitable nucleic acid fragments not only have the above homologies but typically encode a polypeptide having at least 50 amino acids, preferably at least 100 amino acids, more preferably at least 150 amino acids, still more preferably at least 200 amino acids, and most preferably at least 250 amino acids.

The term “codon degeneracy” refers to the nature in the genetic code permitting variation of the nucleotide sequence without affecting the amino acid sequence of an encoded polypeptide. The skilled artisan is well aware of the “codon-bias” exhibited by a specific host cell in usage of nucleotide codons to specify a given amino acid. Therefore, when synthesizing a gene for improved expression in a host cell, it is desirable to design the gene such that its frequency of codon usage approaches the frequency of preferred codon usage of the host cell.

“Synthetic genes” can be assembled from oligonucleotide building blocks that are chemically synthesized using procedures known to those skilled in the art. These oligonucleotide building blocks are annealed and then ligated to form gene segments that are then enzymatically assembled to construct the entire gene. Accordingly, the genes can be tailored for optimal gene expression based on optimization of nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell, where sequence information is available. For example, the codon usage profile for Yarrowia lipolytica is provided in U.S. Pat. No. 7,125,672.

“Gene” refers to a nucleic acid fragment that expresses a specific protein, and which may refer to the coding region alone or may include regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene that is introduced into the host organism by gene transfer. Foreign genes can comprise native genes inserted into a non-native organism, native genes introduced into a new location within the native host, or chimeric genes. A “transgene” is a gene that has been introduced into the genome by a transformation procedure. A “codon-optimized gene” is a gene having its frequency of codon usage designed to mimic the frequency of preferred codon usage of the host cell.

“Coding sequence” refers to a DNA sequence which codes for a specific amino acid sequence. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, enhancers, silencers, 5′ untranslated leader sequence (e.g., between the transcription start site and the translation initiation codon), introns, polyadenylation recognition sequences, RNA processing sites, effector binding sites and stem-loop structures.

“Promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters that cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

The terms “3′ non-coding sequences” and “transcription terminator” refer to DNA sequences located downstream of a coding sequence. This includes polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The 3′ region can influence the transcription, RNA processing or stability, or translation of the associated coding sequence.

“RNA transcript” refers to the product resulting from RNA polymerase-catalyzed transcription of a DNA sequence. When the RNA transcript is a perfect complementary copy of the DNA sequence, it is referred to as the primary transcript or it may be a RNA sequence derived from post-transcriptional processing of the primary transcript and is referred to as the mature RNA. “Messenger RNA” or “mRNA” refers to the RNA that is without introns and which can be translated into protein by the cell. “cDNA” refers to a double-stranded DNA that is complementary to, and derived from, mRNA. “Sense” RNA refers to RNA transcript that includes the mRNA and so can be translated into protein by the cell. “Antisense RNA” refers to a RNA transcript that is complementary to all or part of a target primary transcript or mRNA and that blocks the expression of a target gene (U.S. Pat. No. 5,107,065; Int'l. App. Pub. No. WO 99/28508).

The term “operably linked” refers to the association of nucleic acid sequences on a single nucleic acid fragment so that the function of one is affected by the other. For example, a promoter is operably linked with a coding sequence when it is capable of affecting the expression of that coding sequence. That is, the coding sequence is under the transcriptional control of the promoter. Coding sequences can be operably linked to regulatory sequences in sense or antisense orientation.

The term “recombinant” refers to an artificial combination of two otherwise separated segments of sequence, e.g., by chemical synthesis or by the manipulation of isolated segments of nucleic acids by genetic engineering techniques.

The term “expression”, as used herein, refers to the transcription and stable accumulation of sense (mRNA) or antisense RNA derived from nucleic acid fragments. Expression may also refer to translation of mRNA into a polypeptide. Thus, the term “expression”, as used herein, also refers to the production of a functional end-product (e.g., an mRNA or a protein [either precursor or mature]).

“Transformation” refers to the transfer of a nucleic acid molecule into a host organism, resulting in genetically stable inheritance. The nucleic acid molecule may be a plasmid that replicates autonomously, for example, or, it may integrate into the genome of the host organism.

A “transgenic cell” or “transgenic organism” refers to a cell or organism that contains nucleic acid fragments from a transformation procedure. The transgenic cell or organism may also be are referred to as a “recombinant”, “transformed” or “transformant” cell or organism.

The terms “plasmid” and “vector” refer to an extra chromosomal element often carrying genes that are not part of the central metabolism of the cell, and usually in the form of circular double-stranded DNA fragments. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction that is capable of introducing an expression cassette(s) into a cell.

The term “expression cassette” refers to a fragment of DNA containing a foreign gene and having elements in addition to the foreign gene that allow for enhanced expression of that gene in a foreign host. Generally, an expression cassette will comprise the coding sequence of a selected gene and regulatory sequences preceding (5′ non-coding sequences) and following (3′ non-coding sequences) the coding sequence that are required for expression of the selected gene product. Thus, an expression cassette is typically composed of: 1) a promoter sequence; 2) a coding sequence, i.e., open reading frame [“ORF”]; and, 3) a 3′ untranslated region, i.e., a terminator that in eukaryotes usually contains a polyadenylation site. The expression cassette(s) is usually included within a vector, to facilitate cloning and transformation. Different expression cassettes can be transformed into different organisms including bacteria, yeast, plants and mammalian cells, as long as the correct regulatory sequences are used for each host.

The terms “recombinant construct”, “expression construct” and “construct” are used interchangeably herein. A recombinant construct comprises an artificial combination of nucleic acid fragments, e.g., regulatory and coding sequences that are not found together in nature. For example, a recombinant construct may comprise regulatory sequences and coding sequences that are derived from different sources, or regulatory sequences and coding sequences derived from the same source, but arranged in a manner different than that found in nature. Such a construct may be used by itself or may be used in conjunction with a vector. If a vector is used, then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells comprising any of the isolated nucleic acid fragments described herein. The skilled artisan will also recognize that different independent transformation events will result in different levels and patterns of expression (Jones et al., EMBO J., 4:2411-2418 (1985); De Almeida et al., Mol. Gen. Genetics, 218:78-86 (1989)), and thus that multiple events must be screened in order to obtain strains or lines displaying the desired expression level and pattern.

The term “sequence analysis software” refers to any computer algorithm or software program that is useful for the analysis of nucleotide or amino acid sequences. “Sequence analysis software” may be commercially available or independently developed. Typical sequence analysis software include, but is not limited to: 1) the GCG suite of programs (Wisconsin Package Version 9.0, Genetics Computer Group (GCG), Madison, Wis.); 2) BLASTP, BLASTN, BLASTX (Altschul et al., J. Mol. Biol., 215:403-410 (1990)); 3) DNASTAR (DNASTAR, Inc. Madison, Wis.); 4) Sequencher (Gene Codes Corporation, Ann Arbor, Mich.); and, 5) the FASTA program incorporating the Smith-Waterman algorithm (W. R. Pearson, Comput. Methods Genome Res., [Proc. Int. Symp.] (1994), Meeting Date 1992, 111-20. Editor(s): Suhai, Sandor. Plenum: New York, N.Y.). Within this description, whenever sequence analysis software is used for analysis, the analytical results are based on the “default values” of the program referenced, unless otherwise specified. As used herein “default values” means any set of values or parameters that originally load with the software when first initialized.

Standard recombinant DNA and molecular cloning techniques used herein are well known in the art and are described by Sambrook, J., Fritsch, E. F. and Maniatis, T., Molecular Cloning: A Laboratory Manual, 2^{nd} ed., Cold Spring Harbor Laboratory: Cold Spring Harbor, N.Y. (1989) (hereinafter “Maniatis”); by Silhavy, T. J., Bennan, M. L. and Enquist, L. W., Experiments with Gene Fusions, Cold Spring Harbor Laboratory: Cold Spring Harbor, N.Y. (1984); and by Ausubel, F. M. et al., Current Protocols in Molecular Biology, published by Greene Publishing Assoc. and Wiley-Interscience, Hoboken, N.J. (1987).

The oxidative branch of the pentose phosphate pathway, as described above, comprises three enzymes: glucose-6-phosphate dehydrogenase [“G6PDH”], 6-phosphogluconolactonase [“6PGL”] and 6-phosphogluconate dehydrogenase [“6PGDH”]. However, G6PDH is the rate-limiting enzyme of the PP pathway, allosterically stimulated by NADP⁺ (such that low concentrations of NADP⁺ shunt G-6-P towards glycolysis, while high concentrations of NADP⁺ shunt G-6-P into the PP pathway).

The enzymes of the PP pathway are well studied, particularly G6PDH. This is a result of G6PDH deficiency being the most common human enzyme deficiency in the world, present in more than 400 million people worldwide with the greatest prevalence in people of African, Mediterranean, and Asian ancestry. Specifically, G6PDH deficiency is an X-linked recessive hereditary disease characterized by abnormally low levels of G6PDH and non-immune hemolytic anemia in response to a number of causes, most commonly infection or exposure to certain medications or chemicals. As of 1998, there were almost 100 different known forms of G6PD enzyme molecules encoded by defective G6PD genes, although none were completely inactive—-suggesting that G6PD is indispensable in humans.

Based on the availability of partial and whole genome sequences, numerous gene sequences encoding G6PDH, 6PGL and 6PGDH are publicly available. For example, Tables 3, 4 and 5 present G6PDH, 6PGL and 6PGDH sequences, respectively, having high homology to the G6PDH, 6PGL and 6PGDH proteins of Yarrowia lipolytica. As is well known in the art, these may be used to readily search for G6PDH, 6PGL and/or 6PGDH homologs, respectively, in the same or other species using sequence analysis software. In general, such computer software matches similar sequences by assigning degrees of homology to various substitutions, deletions, and other modifications. Use of software algorithms, such as the BLASTP method of alignment with a low complexity filter and the following parameters: Expect value=10, matrix=Blosum 62 (Altschul, et al., Nucleic Acids Res., 25:3389-3402 (1997)), is well-known for comparing any G6PDH, 6PGL and/or 6PGDH protein in Table 3, Table 4 or Table 5 against a database of nucleic or protein sequences and thereby identifying similar known sequences within a preferred organism.

Use of a software algorithm to comb through databases of known sequences is particularly suitable for the isolation of homologs having a relatively low percent identity to publicly available G6PDH, 6PGL and/or 6PGDH sequences, such as those described in Table 3, Table 4 and Table 5, respectively. It is predictable that isolation would be relatively easier for G6PDH, 6PGL and/or 6PGDH homologs of at least about 70%-85% identity to publicly available G6PDH, 6PGL and/or 6PGDH sequences. Further, those sequences that are at least about 85%-90% identical would be particularly suitable for isolation and those sequences that are at least about 90%-95% identical would be the most easily isolated.

Some G6PDH homologs have also been isolated by the use of motifs unique to G6PDH enzymes. For example, it is well known that G6PDH possesses NADP⁺ binding motifs (Levy, H., et al., Arch. Biochem. Biophys., 326:145-151 (1996)). These regions of “conserved domain” correspond to a set of amino acids that are highly conserved at specific positions, which likely represent a region of the G6PDH protein that is essential to the structure, stability or activity of the protein. Motifs are identified by their high degree of conservation in aligned sequences of a family of protein homologues. As unique “signatures”, they can determine if a protein with a newly determined sequence belongs to a previously identified protein family. These motifs are useful as diagnostic tools for the rapid identification of novel G6PDH genes.

Alternatively, the publicly available G6PDH, 6PGL and/or 6PGDH sequences or their motifs may be hybridization reagents for the identification of homologs. The basic components of a nucleic acid hybridization test include a probe, a sample suspected of containing the gene or gene fragment of interest, and a specific hybridization method. Probes are typically single-stranded nucleic acid sequences that are complementary to the nucleic acid sequences to be detected. Probes are hybridizable to the nucleic acid sequence to be detected. Although probe length can vary from 5 bases to tens of thousands of bases, typically a probe length of about 15 bases to about 30 bases is suitable. Only part of the probe molecule need be complementary to the nucleic acid sequence to be detected. In addition, the complementarity between the probe and the target sequence need not be perfect. Hybridization does occur between imperfectly complementary molecules with the result that a certain fraction of the bases in the hybridized region are not paired with the proper complementary base.

Hybridization methods are well known. Typically the probe and the sample must be mixed under conditions that permit nucleic acid hybridization. This involves contacting the probe and sample in the presence of an inorganic or organic salt under the proper concentration and temperature conditions. The probe and sample nucleic acids must be in contact for a long enough time that any possible hybridization between the probe and the sample nucleic acid occurs. The concentration of probe or target in the mixture determine the time necessary for hybridization to occur. The higher the concentration of the probe or target, the shorter the hybridization incubation time needed. Optionally, a chaotropic agent may be added, such as guanidinium chloride, guanidinium thiocyanate, sodium thiocyanate, lithium tetrachloroacetate, sodium perchlorate, rubidium tetrachloroacetate, potassium iodide or cesium trifluoroacetate. If desired, one can add formamide to the hybridization mixture, typically 30-50% (v/v) [“by volume”].

Various hybridization solutions can be employed. Typically, these comprise from about 20 to 60% volume, preferably 30%, of a polar organic solvent. A common hybridization solution employs about 30-50% v/v formamide, about 0.15 to 1 M sodium chloride, about 0.05 to 0.1 M buffers (e.g., sodium citrate, Tris-HCl, PIPES or HEPES (pH range about 6-9)), about 0.05 to 0.2% detergent (e.g., sodium dodecylsulfate), or between 0.5-20 mM EDTA, FICOLL (Pharmacia Inc.) (about 300-500 kdal), polyvinylpyrrolidone (about 250-500 kdal), and serum albumin. Also included in the typical hybridization solution are unlabeled carrier nucleic acids from about 0.1 to 5 mg/mL, fragmented nucleic DNA such as calf thymus or salmon sperm DNA or yeast RNA, and optionally from about 0.5 to 2% wt/vol [“weight by volume”] glycine. Other additives may be included, such as volume exclusion agents that include polar water-soluble or swellable agents (e.g., polyethylene glycol), anionic polymers (e.g., polyacrylate or polymethylacrylate) and anionic saccharidic polymers, such as dextran sulfate.

Nucleic acid hybridization is adaptable to a variety of assay formats. One of the most suitable is the sandwich assay format. The sandwich assay is particularly adaptable to hybridization under non-denaturing conditions. A primary component of a sandwich-type assay is a solid support. The solid support has adsorbed or covalently coupled to it immobilized nucleic acid probe that is unlabeled and complementary to one portion of the sequence.

Any of the G6PDH, 6PGL and/or 6PGDH nucleic acid fragments described herein or in public literature, or any identified homologs, may be used to isolate genes encoding homologous proteins from the same or other species. Isolation of homologous genes using sequence-dependent protocols is well known in the art. Examples of sequence-dependent protocols include, but are not limited to: 1) methods of nucleic acid hybridization; 2) methods of DNA and RNA amplification, as exemplified by various uses of nucleic acid amplification technologies, such as polymerase chain reaction [“PCR”] (U.S. Pat. No. 4,683,202); ligase chain reaction [“LCR”] (Tabor, S. et al., Proc. Natl. Acad. Sci. U.S.A., 82:1074 (1985)); or strand displacement amplification [“SDA”] (Walker, et al., Proc. Natl. Acad. Sci. U.S.A., 89:392 (1992)); and, 3) methods of library construction and screening by complementation.

For example, genes encoding proteins or polypeptides similar to publicly available G6PDH, 6PGL and/or 6PGDH genes or their motifs could be isolated directly by using all or a portion of those publicly available nucleic acid fragments as DNA hybridization probes to screen libraries from any desired organism using well known methods. Specific oligonucleotide probes based upon the publicly available nucleic acid sequences can be designed and synthesized by methods known in the art (Maniatis, supra). Moreover, the entire sequences can be used directly to synthesize DNA probes by methods known to the skilled artisan, such as random primers DNA labeling, nick translation or end-labeling techniques, or RNA probes using available in vitro transcription systems. In addition, specific primers can be designed and used to amplify a part or the full length of the publicly available sequences or their motifs. The resulting amplification products can be labeled directly during amplification reactions or labeled after amplification reactions, and used as probes to isolate full-length DNA fragments under conditions of appropriate stringency.

Based on any of the well-known methods just discussed, it would be possible to identify and/or isolate G6PDH, 6PGL and/or 6PGDH gene homologs in any preferred organism of choice.

Most anabolic processes in the cell, wherein complex molecules are synthesized from smaller units, are powered by either adenosine triphosphate [“ATP”] or NADPH. With respect to NADPH, the oxidative phase of the PP pathway is the major source of NADPH in cells, producing approximately 60% of the NADPH required. Thus, the reactions catalyzed by G6PDH, 6PGL and 6PGDH play a significant role in cellular metabolism, based on their ability to generate cellular NADPH. This molecule then provides the reducing equivalents for numerous anabolic pathways.

The instant invention relates to increasing intracellular availability of NADPH, thereby allowing for increased production of non-native products that require this cofactor in their biosynthetic pathways. More specifically, described herein is a method for the production of a non-native product of interest comprising:
(a) providing a transgenic microorganism comprising:
   (i) at least one gene encoding glucose-6-phosphate dehydrogenase [“G6PDH”];
   (ii) at least one gene encoding 6-phosphogluconolactonase [“6PGL”]; and,
   (iii) at least one heterologous gene encoding a non-native product of interest;
      wherein biosynthesis of the non-native product of interest comprises at least one enzymatic reaction that requires nicotinamide adenine dinucleotide phosphate [“NADPH”]; and,
      wherein (i) and (ii) are over-expressed in a coordinately regulated fashion; and,
      wherein an increased quantity of NADPH is produced when compared to the quantity of NADPH produced by a transgenic microorganism either lacking or not over-expressing (i) and (ii) in a coordinately regulated fashion;
(b) growing the transgenic microorganism of step (a) in the presence of a fermentable carbon source whereby expression of (iii) results in production of the non-native product of interest; and,
(c) optionally recovering the non-native product of interest.

More specifically, the at least one gene encoding G6PDH and the at least one gene encoding 6PGL are over-expressed in a coordinately regulated fashion, which may be achieved by a means selected from the group consisting of:
(a) operable linkage of the at least one gene encoding G6PDH to a first promoter and operable linkage of the at least one gene encoding 6PGL to a second promoter, wherein the first promoter has equivalent or reduced activity when compared to the second promoter [i.e., the first promoter and the second promoter may be the same or different from one another];
(b) expression of the at least one gene encoding G6PDH in multicopy and expression of the at least one gene encoding 6PGL in multicopy, wherein the copy number of the at least one gene encoding G6PDH is equivalent or reduced when compared to the copy number of the at least one gene encoding 6PGL;
(c) linkage of the enzymatic activity of the at least one gene encoding G6PDH to the enzymatic activity of the at least one gene encoding 6PGL via creation of a multizyme; and,
(d) a combination of any of the means set forth in (a), (b) and (c).

Over-expression of biosynthetic routes comprising at least one NADPH-dependent reaction will dramatically increase the level of NADP⁺, thus stimulating G6PDH to produce additional NADPH.

In some embodiments of the methods described above, further increase in cellular availability of NADPH may be obtained by additionally expressing 6PGDH.

Any non-native product of interest possessing at least one NADPH-dependent reaction can be produced using the transgenic microorganism and/or method of the instant invention. Examples of such non-native products that possess NADPH-dependent reactions include, but are not limited to, polyunsaturated fatty acids, carotenoids, quinoines, stilbenes, vitamins, sterols, flavonoids, organic acids, polyols and hydroxyesters.

More specifically, in lipid synthesis, NADPH is required for fatty acid biosynthesis. Specifically, for example, synthesis of one molecule of the polyunsaturated fatty acid linoleic acid [“LA”, 18:2 ω-6] requires at least 16 molecules of NADPH, as illustrated in the following reaction: 9 acetyl-CoA+8 ATP+16 NADPH+2 NADH→LA+8 ADP+16 NADP⁺+2 NAD. Thus, lipid synthesis is dependent on cellular availability of NADPH. The term “fatty acids” refers to long chain aliphatic acids (alkanoic acids) of varying chain lengths, from about C₁₂ to C₂₂, although both longer and shorter chain-length acids are known. The predominant chain lengths are between C₁₆ and C₂₂. The structure of a fatty acid is represented by a simple notation system of “X:Y”, where X is the total number of carbon [“C”] atoms in the particular fatty acid and Y is the number of double bonds.

Additional details concerning the differentiation between “saturated fatty acids” versus “unsaturated fatty acids”, “monounsaturated fatty acids” versus “polyunsaturated fatty acids” [“PUFAs”], and “omega-6 fatty acids” [“n-6”] versus “omega-3 fatty acids” [“n-3”] are provided in U.S. Pat. No. 7,238,482, which is hereby incorporated herein by reference. U.S. Pat. App. Pub. No. 2009-0093543-A1, Table 3, provides a detailed summary of the chemical and common names of omega-3 and omega-6 PUFAs and their precursors, and well as commonly used abbreviations.

Some examples of PUFAs, however, include, but are not limited to, linoleic acid [‘LA”, 18:2 ω-6], gamma-linolenic acid [“GLA”, 18:3 ω-6], eicosadienoic acid [“EDA”, 20:2 ω-6], dihomo-gamma-linolenic acid [“GLA”, 20:3 ω-6], arachidonic acid [“ARA”, 20:4 ω-6], docosatetraenoic acid [“DTA”, 22:4 ω-6], docosapentaenoic acid [“DPAn-6”, 22:5 ω-6], alpha-linolenic acid [“ALA”, 18:3 ω-3], stearidonic acid [“STA”, 18:4 ω-3], eicosatrienoic acid [“ETA”, 20:3 ω-3], eicosatetraenoic acid [“ETrA”, 20:4 ω-3], eicosapentaenoic acid [“EPA”, 20:5 ω-3], docosapentaenoic acid [“DPAn-3”, 22:5 ω-3] and docosahexaenoic acid [“DHA”, 22:6 ω-3].

As a further example of the need for NADPH in PUFA biosynthesis, EPA biosynthesis from glucose can be expressed by the following chemical equations:

glucose+2ADP+4NAD→2 acetyl-CoA+2ATP+4NADH+2CO₂ (Equation 1)

10 acetyl-CoA+9ATP+18NADPH+5NADH→EPA+9ADP+18NADP⁺+5NAD (Equation 2)

In cholesterol synthesis, NADPH is required for reduction reactions and thus multiple moles of NADPH are required for synthesis of one mole of cholesterol. Thus, biosynthesis of sterols is dependent on cellular availability of NADPH. Examples of sterol compounds includes: squalene, lanosterol, zymosterol, ergosterol, 7-dehydrocholesterol (provitamin D3), and combinations thereof.

Similarly, in isoprenoid biosynthesis, NADPH is required as an electron donor for the reduction reactions. For example, two moles of NADPH are required for the conversion of HMG-CoA to mevalonate, which is the precursor to isoprene. Further conversion of isoprene to other isoprenoids also requires additional NADPH for the reduction/desaturation steps. The term “isoprenoid compound” refers to compounds formally derived from isoprene (2-methylbuta-1,3-diene; CH₂═C(CH₃)CH═CH₂), the skeleton of which can generally be discerned in repeated occurrence in the molecule. These compounds are produced biosynthetically via the isoprenoid pathway beginning with isopentenyl pyrophosphate and formed by the head-to-tail condensation of isoprene units, leading to molecules which may be, for example, of 5, 10, 15, 20, 30, or 40 carbons in length. Isoprenoid compounds include, for example: terpenes, terpenoids, carotenoids, quinone derived compounds, dolichols, and squalene; thus, biosynthesis of all of these compounds is dependent on cellular availability of NADPH.

As used herein, the term “carotenoid” refers to a class of hydrocarbons having a conjugated polyene carbon skeleton formally derived from isoprene. This class of molecules is composed of triterpenes [“C₃₀ diapocarotenoids”] and tetraterpenes [“C₄₀ carotenoids”] and their oxygenated derivatives; and, these molecules typically have strong light absorbing properties and may range in length in excess of C₂₀₀. Other “carotenoid compounds” are known which are C₃₅, C₅₀, C₆₀, C₇₀ and C₈₀ in length, for example. The term “carotenoid” may include both carotenes and xanthophylls. A “carotene” refers to a hydrocarbon carotenoid (e.g., phytoene, β-carotene and lycopene). In contrast, the term “xanthophyll” refers to a C₄₀ carotenoid that contains one or more oxygen atoms in the form of hydroxy-, methoxy-, oxo-, epoxy-, carboxy-, or aldehydic functional groups. Xanthophylls are more polar than carotenes and this property dramatically reduces their solubility in fats and lipids. Thus, suitable examples of carotenoids include: antheraxanthin, adonirubin, adonixanthin, astaxanthin (i.e., 3,3′-dihydroxy-β,β-carotene-4,4′-dione), canthaxanthin (i.e., β,β-carotene-4,4′-dione), capsorubrin, β-cryptoxanthin, α-carotene, β,ψ-carotene, δ-carotene, ε-carotene, β-carotene keto-γ-carotene, echinenone, 3-hydroxyechinenone, 3′-hydroxyechinenone, γ-carotene, ψ-carotene, ζ-carotene, zeaxanthin, adonirubin, tetrahydroxy-β,β′-caroten-4,4′-dione, tetrahydroxy-β,β′-caroten-4-one, caloxanthin, erythroxanthin, nostoxanthin, flexixanthin, 3-hydroxy-γ-carotene, 3-hydroxy-4-keto-γ-carotene, bacteriorubixanthin, bacteriorubixanthinal, lutein, 4-keto-γ-carotene, α-cryptoxanthin, deoxyflexixanthin, diatoxanthin, 7,8-didehydroastaxanthin, didehydrolycopene, fucoxanthin, fucoxanthinol, isorenieratene, β-isorenieratene, lactucaxanthin, lutein, lycopene, myxobactone, neoxanthin, neurosporene, hydroxyneurosporene, peridinin, phytoene, phytofluene, rhodopin, rhodopin glucoside, 4-keto-rubixanthin, siphonaxanthin, spheroidene, spheroidenone, spirilloxanthin, torulene, 4-keto-torulene, 3-hydroxy-4-keto-torulene, uriolide, uriolide acetate, violaxanthin, zeaxanthin-β-diglucoside, and combinations thereof.

The term “at least one quinone derived compound” refers to compounds having a redox-active quinone ring structure and includes compounds selected from the group consisting of: quinones of the CoQ series (i.e., that is Q₆, Q₇, Q₈, Q₉ and Q₁₀), vitamin K compounds, vitamin E compounds, and combinations thereof. For example, the term coenzyme Q₁₀ [“CoQ_{10″}”] refers to 2,3-dimethoxy-dimethyl-6-decaprenyl-1,4-benzoquinone, also known as ubiquinone-10 (CAS Registry No. 303-98-0). The benzoquinone portion of CoQ₁₀ is synthesized from tyrosine, whereas the isoprene sidechain is synthesized from acetyl-CoA through the mevalonate pathway. Thus, biosynthesis of CoQ compounds such as CoQ₁₀ requires NADPH. A “vitamin K compound” includes, e.g., menaquinone or phylloquinone, while a vitamin E compound includes, e.g., tocopherol, tocotrienol or an α-tocopherol.

In resveratrol biosynthesis, NADPH is required for the production of the aromatic precursor tyrosine. Thus, resveratrol [“3,4′,5-trihydroxystilbene”] biosynthesis is dependent on cellular availability of NADPH.

One of skill in the art could readily generate examples of other products of interest possessing at least one NADPH-dependent reaction. The present examples are not intended to be limiting and it should be clear that alternate products are also contemplated.

Any microorganism capable of being engineered to produce a non-native product of interest can be used to practice the invention. Examples of such microorganisms include, but are not limited to, various bacteria, algae, yeast, euglenoids, stramenopiles, oomycetes and fungi. These microorganisms are characterized as comprising at least one heterologous gene that enables biosynthesis of the non-native product of interest, prior to coordinately regulating over-expression of G6PDH and 6PGL as described herein. Alternatively, it is to be understood that one could manipulate the microorganism to coordinately regulate over-expression of G6PDH and 6PGL first and then introduce the at least one heterologous gene that enables biosynthesis of the non-native product of interest subsequently or the transformations could be performed simultaneously to accomplish the same end result.

In some cases, oleaginous organisms may be preferred if the product of interest is lipophilic. Oleaginous organisms are naturally capable of oil synthesis and accumulation, commonly accumulating in excess of about 25% of their dry cell weight as oil. Various algae, moss, fungi, yeast, stramenopiles and plants are naturally classified as oleaginous. More preferred are oleaginous yeasts; genera typically identified as oleaginous yeast include, but are not limited to: Yarrowia, Candida, Rhodotorula, Rhodosporidium, Cryptococcus, Trichosporon and Lipomyces. More specifically, illustrative oil-synthesizing yeasts include: Rhodosporidium toruloides, Lipomyces starkeyii, L. lipoferus, Candida revkaufi, C. pulcherrima, C. tropicalis, C. utilis, Trichosporon pullans, T. cutaneum, Rhodotorula glutinus, R. graminis and Yarrowia lipolytica (formerly classified as Candida lipolytica). The most preferred oleaginous yeast is Yarrowia lipolytica; and most preferred are Y. lipolytica strains designated as ATCC #76982, ATCC #20362, ATCC #8862, ATCC #18944 and/or LGAM S(7)1 (Papanikolaou S., and Aggelis G., Bioresour. Technol., 82(1):43-9 (2002)). In alternate embodiments, a non-oleaginous organism can be genetically modified to become oleaginous, e.g., yeast such as Saccharomyces cerevisiae (Int'l. App. Pub. No. WO 2006/102342).

Thus, for example, numerous microorganisms have been genetically engineered to produce long-chain PUFAs, by introduction of the appropriate combination of desaturase (i.e., delta-12 desaturase, delta-6 desaturase, delta-8 desaturase, delta-5 desaturase, delta-17 desaturase, delta-15 desaturase, delta-9 desaturase, delta-4 desaturase) and elongase (i.e., C_{14/16} elongase, C_{16/18} elongase, C_{18/20} elongase, C_{20/22} elongase and delta-9 elongase) genes. See, for example, work in Saccharomyces cerevisiae (Dyer, J. M. et al., Appl. Eniv. Microbiol., 59:224-230 (2002); Domergue, F. et al., Eur. J. Biochem., 269:4105-4113 (2002); U.S. Pat. No. 6,136,574; U.S. Pat. Appl. Pub. No. 2006-0051847-A1), in the marine cyanobacterium Synechococcus sp. (Yu, R., et al., Lipids, 35(10):1061-1064 (2006)), in the methylotrophic yeast Pichia pastoris (Kajikawa, M. et al., Plant Mol. Biol., 54(3):335-52 (2004)) and in the moss Physcomitrella patens (Kaewsuwan, S., et al., Bioresour. Technol., 101(11):4081-4088 (2010)).

Tremendous effort has also been invested towards engineering strains of the oleaginous yeast, Yarrowia lipolytica, for PUFA production, as described in the following references, hereby incorporated herein by reference in their entirety: U.S. Pat. No. 7,238,482; U.S. Pat. No. 7,465,564; U.S. Pat. No. 7,588,931; U.S. Pat. Appl. Pub. No. 2006-0115881-A1; U.S. Pat. No. 7,550,286; U.S. Pat. Appl. Pub. No. 2009-0093543-A1; U.S. Pat. Appl. Pub. No. 2010-0317-072 A1.

In each of these recombinant organisms engineered for PUFA biosynthesis, supra, it would be expected that coordinately regulated over-expression of G6PDH and 6PGL would result in an increased quantity of NADPH, thereby permitting an increased quantity of the PUFAs to be produced (as compared to a similarly engineered recombinant organism that is not over-expressing G6PDH and 6PGL in a coordinately regulated fashion).

In some embodiments wherein the microorganism is an oleaginous yeast and the non-native product of interest is a PUFA, the coordinately regulated over-expression of G6PDH and 6PGL will also result in increased the total lipid content (in addition to increased production of PUFAs).

In alternate embodiments, the microorganism may be manipulated for a variety of purposes to produce alternate non-native products of interest. For example, wildtype Yarrowia lipolytica is not normally carotenogenic and does not produce resveratrol, although it can natively produce coenzyme Q₉ and ergosterol. Int'l. App. Pub. No. WO 2008/073367 and Int'l. App. Pub. No. WO 2009/126890 describe the production of a suite of carotenoids in Y. lipolytica via introduction of carotenoid biosynthetic pathway genes, such as crtE encoding a geranyl geranyl pyrophosphate synthase, crtB encoding phytoene synthase, crtl encoding phytoene desaturase, crtY encoding lycopene cyclase, crtZ encoding carotenoid hydroxylase and/or crtW encoding carotenoid ketolase.

U.S. Pat. App. Pub. No. 2009/0142322-A1 and WO 2007/120423 describe production of various quinone derived compounds in Y. lipolytica via introduction of heterologous quinone biosynthetic pathway genes, such as ddsA encoding decaprenyl diphosphate synthase for production of coenzyme Q₁₀, genes encoding the MenF, MenD, MenC, MenE, MenB, MenA, UbiE, and/or MenG polypeptides for production of vitamin K compounds, and genes encoding the tyrA, pdsl(hppd), VTEI, HPT1 (VTE2), VTE3, VTE4, and/or GGH polypeptides for production of vitamin E compounds, etc. Int'l. App. Pub. No. WO 2008/130372 describes production of sterols in Y. lipolytica via introduction of ERG9/SQS1 encoding squalene synthase and ERG encoding squalene epoxidase. And, Int'l. App. Pub. No. WO 2006/125000 describes production of resveratrol in Y. lipolytica via introduction of a gene encoding resveratrol synthase.

In each of these recombinant organisms engineered for production of a non-native product, it would be expected that coordinately regulated over-expression of G6PDH and 6PGL would result in an increased quantity of NADPH, thereby permitting an increased quantity of the product (i.e., PUFAs, carotenoids, quinine derived compounds, vitamin K compounds, vitamin E compounds, sterols, resveratrol), as compared to a similarly engineered recombinant organism that is not over-expressing G6PDH and 6PGL in a coordinately regulated fashion.

One of ordinary skill in the art is well aware of other transgenic microorganisms that have been engineered to produce a variety of non-native products of interest and any of these are suitable for use in the disclosure herein, provided that at least one of the biosynthetic reactions leading to production of the non-native product is dependent on NADPH.

In another aspect the instant invention concerns a transgenic microorganism comprising:
(a) at least one gene encoding glucose-6-phosphate dehydrogenase [“G6PDH”];
(b) at least one gene encoding 6-phosphogluconolactonase [“6PGL”]; and,
(c) at least one heterologous gene encoding a non-native product of interest;

wherein biosynthesis of the non-native product of interest comprises at least one enzymatic reaction that requires nicotinamide adenine dinucleotide phosphate [“NADPH”]; and,

wherein coordinately regulated over-expression of (a) and (b) results in an increased quantity of NADPH; and,

wherein the increased quantity of NADPH results in an increased quantity of the product of interest produced by expression of (c) in the transgenic microorganism;

when compared to the quantity of NADPH and the quantity of the product of interest produced by a transgenic microorganism comprising (c) and either lacking or not over-expressing (a) and (b) in a coordinately regulated fashion.

In preferred embodiments, coordinately regulated over-expression of the at least one gene encoding G6PDH and the at least one gene encoding 6PGL is achieved by a means selected from the group consisting of:
(a) the at least one gene encoding G6PDH is operably linked to a first promoter and the at least one gene encoding 6PGL is operably linked to a second promoter, wherein the first promoter has equivalent or reduced activity when compared to the second promoter;
(b) the at least one gene encoding G6PDH is expressed in multicopy and the at least one gene encoding 6PGL is expressed in multicopy, wherein the copy number of the at least one gene encoding G6PDH is equivalent or reduced when compared to the copy number of the at least one gene encoding 6PGL;
(c) the enzymatic activity of the at least one gene encoding G6PDH is linked to the enzymatic activity of the at least one gene encoding 6PGL as a multizyme; and,
(d) a combination of any of the means set forth in (a), (b) and (c).

In some embodiments, the transgenic microorganism also expresses at least one gene encoding 6-phosphogluconate dehydrogenase, in addition to the genes of (a), (b) and (c).

It is necessary to create and introduce a recombinant construct(s) comprising at least one open reading frame [“ORF”] encoding a PP pathway gene into a host microorganism comprising at least one heterologous gene encoding a non-native product of interest. One of skill in the art is aware of standard resource materials that describe: 1) specific conditions and procedures for construction, manipulation and isolation of macromolecules, such as DNA molecules, plasmids, etc.; 2) generation of recombinant DNA fragments and recombinant expression constructs; and, 3) screening and isolating of clones. See Sambrook et al., Molecular Cloning: A Laboratory Manual, 2^{nd} ed., Cold Spring Harbor Laboratory: Cold Spring Harbor, N.Y. (1989); Maliga et al., Methods in Plant Molecular Biology, Cold Spring Harbor, N.Y. (1995); Birren et al., Genome Analysis: Detecting Genes, v. 1, Cold Spring Harbor, N.Y. (1998); Birren et al., Genome Analysis: Analyzing DNA, v. 2, Cold Spring Harbor: NY (1998); Plant Molecular Biology: A Laboratory Manual, Clark, ed. Springer: NY (1997).

In general, the choice of sequences included in a construct depends on the desired expression products, the nature of the host cell and the proposed means of separating transformed cells versus non-transformed cells. The skilled artisan is aware of the genetic elements that must be present on the plasmid vector to successfully transform, select and propagate host cells containing the chimeric gene. Typically, however, the vector or cassette contains sequences directing transcription and translation of the relevant gene(s), a selectable marker and sequences allowing autonomous replication or chromosomal integration. Suitable vectors comprise a region 5′ of the gene that controls transcriptional initiation, i.e., a promoter, and a region 3′ of the DNA fragment that controls transcriptional termination, i.e., a terminator. It is most preferred when both control regions are derived from genes from the transformed host cell.

Initiation control regions or promoters useful for driving expression of heterologous genes or portions of them in the desired host cell are numerous and well known. These control regions may comprise a promoter, enhancer, silencer, intron sequences, 3′ UTR and/or 5′ UTR regions, and protein and/or RNA stabilizing elements. Such elements may vary in their strength and specificity. Virtually any promoter, i.e., native, synthetic, or chimeric, capable of directing expression of these genes in the selected host cell is suitable. Expression in a host cell can occur in an induced or constitutive fashion. Induced expression occurs by inducing the activity of a regulatable promoter operably linked to the gene of interest. Constitutive expression occurs by the use of a constitutive promoter operably linked to the gene of interest. One of skill in the art will readily be able to discern strength of activity of a first promoter relative to that of a second promoter, using means well known to those of skill in the art.

When the host microorganism is, e.g., yeast, transcriptional and translational regions functional in yeast cells are provided, particularly from the host species. See, for example, Int'l. App. Pub. No. WO 2006/052870 and U.S. Pat. Pub. No. 2009-009-3543-A1 for preferred transcriptional initiation regulatory regions for use in Yarrowia lipolytica. Any number of regulatory sequences may be used, depending on whether constitutive or induced transcription is desired, the efficiency of the promoter in expressing the ORF of interest, the ease of construction, etc.

3′ non-coding sequences encoding transcription termination signals, i.e., a “termination region”, must be provided in a recombinant construct and may be from the 3′ region of the gene from which the initiation region was obtained or from a different gene. A large number of termination regions are known and function satisfactorily in a variety of hosts when utilized in both the same and different genera and species from which they were derived. The termination region is selected more for convenience rather than for any particular property. Termination regions may also be derived from various genes native to the preferred hosts.

Particularly useful termination regions for use in yeast are derived from a yeast gene, particularly Saccharomyces, Schizosaccharomyces, Candida, Yarrowia or Kluyveromyces. The 3′-regions of mammalian genes encoding γ-interferon and α-2 interferon are also known to function in yeast. The 3′-region can also be synthetic, as one of skill in the art can utilize available information to design and synthesize a 3′-region sequence that functions as a transcription terminator. A termination region may be unnecessary, but is highly preferred.

The vector may comprise a selectable and/or scorable marker, in addition to the regulatory elements described above. Preferably, the marker gene is an antibiotic resistance gene such that treating cells with the antibiotic results in growth inhibition, or death, of untransformed cells and uninhibited growth of transformed cells. For selection of yeast transformants, any marker that functions in yeast is useful with resistance to kanamycin, hygromycin and the amino glycoside G418 and the ability to grow on media lacking uracil, lysine, histine or leucine being particularly useful.

Merely inserting a gene into a cloning vector does not ensure its expression at the desired rate, concentration, amount, etc. In response to the need for a high expression rate, many specialized expression vectors have been created by manipulating a number of different genetic elements that control transcription, RNA stability, translation, protein stability and location, oxygen limitation, and secretion from the host cell. Some of the manipulated features include: the nature of the relevant transcriptional promoter and terminator sequences, the number of copies of the cloned gene and whether the gene is plasmid-borne or integrated into the genome of the host cell, the final cellular location of the synthesized foreign protein, the efficiency of translation and correct folding of the protein in the host organism, the intrinsic stability of the mRNA and protein of the cloned gene within the host cell and the codon usage within the cloned gene, such that its frequency approaches the frequency of preferred codon usage of the host cell. Each of these may be used in the methods and host cells described herein to further optimize expression of PP pathway genes.

In particular, coordinately regulated over-expression is required in the present invention for the at least one gene encoding G6PDH and the at least one gene encoding 6PGL. One method by which this can be accomplished is via ensuring that the gene encoding G6PDH is operably linked to a first promoter and the gene encoding 6PGL is operably linked to a second promoter, wherein the first promoter has equivalent or reduced activity which compared to the second promoter. In some cases, the first promoter and the second promoter are the same. This allows similar amounts of 6PGL and G6PDH activity in the cell, such that a balanced flux through the PP pathway is maintained.

As one of skill in the art is aware, a variety of methods are available to compare the activity of various promoters. This type of comparison is useful to facilitate a determination of each promoter's strength. Thus, it may be useful to indirectly quantitate promoter activity based on reporter gene expression (i.e., the E. coli gene encoding β-glucuronidase (GUS), wherein GUS activity in each expressed construct may be measured by histochemical and/or fluorometric assays (Jefferson, R. A. Plant Mol. Biol. Reporter 5:387-405 (1987)). In alternate embodiments, it may sometimes be useful to quantify promoter activity using more quantitative means. One suitable method is the use of real-time PCR (for a general review of real-time PCR applications, see Ginzinger, D. J., Experimental Hematology, 30:503-512 (2002)). Real-time PCR is based on the detection and quantitation of a fluorescent reporter. This signal increases in direct proportion to the amount of PCR product in a reaction. By recording the amount of fluorescence emission at each cycle, it is possible to monitor the PCR reaction during exponential phase where the first significant increase in the amount of PCR product correlates to the initial amount of target template. There are two general methods for the quantitative detection of the amplicon: (1) use of fluorescent probes; or (2) use of DNA-binding agents (e.g., SYBR-green I, ethidium bromide). For relative gene expression comparisons, it is necessary to use an endogenous control as an internal reference (e.g., a chromosomally encoded 16S rRNA gene), thereby allowing one to normalize for differences in the amount of total DNA added to each real-time PCR reaction. Specific methods for real-time PCR are well documented in the art. See, for example, the Real Time PCR Special Issue (Methods, 25(4):383-481 (2001)).

Following a real-time PCR reaction, the recorded fluorescence intensity is used to quantitate the amount of template by use of: 1) an absolute standard method (wherein a known amount of standard such as in vitro translated RNA (cRNA) is used); 2) a relative standard method (wherein known amounts of the target nucleic acid are included in the assay design in each run); or 3) a comparative C_{T} method (ΔΔC_{T}) for relative quantitation of gene expression (wherein the relative amount of the target sequence is compared to any of the reference values chosen and the result is given as relative to the reference value). The comparative C_{T} method requires one to first determine the difference (ΔC_{T}) between the C_{T} values of the target and the normalizer, wherein: ΔC_{T}=C_{T} (target)−C_{T} (normalizer). This value is calculated for each sample to be quantitated and one sample must be selected as the reference against which each comparison is made. The comparative ΔΔC_{T} calculation involves finding the difference between each sample's ΔC_{T} and the baseline's ΔC_{T}, and then transforming these values into absolute values according to the formula 2^{-ΔΔCT}.

Although not to be considered limiting to the invention herein, Int'l. App. Pub. No. WO 2006/2006/052870 does provide examples of means to directly compare the activity of seven different promoters in Yarrowia lipolytica, under comparable conditions.

After a recombinant construct is created comprising at least one chimeric gene comprising a promoter, a PP pathway ORF and a terminator, it is placed in a plasmid vector capable of autonomous replication in the host microorganism or is directly integrated into the genome of the host microorganism. Integration of expression cassettes can occur randomly within the host genome or can be targeted through the use of constructs containing regions of homology with the host genome sufficient to target recombination with the host locus. Where constructs are targeted to an endogenous locus, all or some of the transcriptional and translational regulatory regions can be provided by the endogenous locus.

When two or more genes are expressed from separate replicating vectors, each vector may have a different means of selection and should lack homology to the other construct(s) to maintain stable expression and prevent reassortment of elements among constructs. Judicious choice of regulatory regions, selection means and method of propagation of the introduced construct(s) can be experimentally determined so that all introduced genes are expressed at the necessary levels to provide for synthesis of the desired products.

Constructs comprising the gene of interest may be introduced into a host cell by any standard technique. These techniques include transformation, e.g., lithium acetate transformation (Methods in Enzymology, 194:186-187 (1991)), protoplast fusion, biolistic impact, electroporation, microinjection, vacuum filtration or any other method that introduces the gene of interest into the host cell.

For convenience, a host microorganism that has been manipulated by any method to take up a DNA sequence, for example, in an expression cassette, is referred to herein as “transformed” or “recombinant”. The transformed host will have at least one copy of the expression construct and may have two or more, depending upon whether the gene is integrated into the genome, amplified, or is present on an extrachromosomal element having multiple copy numbers.

An alternate means to achieve coordinately regulated over-expression of the at least one gene encoding G6PDH and the at least one gene encoding 6PGL occurs when the genes are expressed in multicopy. Specifically, if the copy number of the at least one gene encoding G6PDH is equivalent or reduced with respect to the copy number of the at least one gene encoding 6PGL, this allows similar amounts of 6PGL and G6PDH activity in the cell such that a balanced flux through the PP pathway is maintained.

Or, one of skill in the art could also ensure coordinately regulated over-expression of the at least one gene encoding G6PDH and the at least one gene encoding 6PGL by creating a multizyme comprising both enzymes. Int'l. App. Pub. No. WO 2008/124048 teaches means to link at least two independent and separable enzymatic activities in a single polypeptide as a “multizyme” or “fusion protein”. Appropriate bonds or links between the two or more polypeptides each having independent and separable enzymatic activities are also included therein and thus creation of a G6PDH-6PGL multizyme would be facile. This approach would also be suitable to ensure that similar amounts of 6PGL and G6PDH activity in the cell were obtained, thereby maintaining a balanced flux through the PP pathway.

The transformed host microorganism can be identified by selection for a marker contained on the introduced construct. Alternatively, a separate marker construct may be co-transformed with the desired construct, as many transformation techniques introduce many DNA molecules into host cells.

Typically, transformed hosts are selected for their ability to grow on selective media, which may incorporate an antibiotic or lack a factor necessary for growth of the untransformed host, such as a nutrient or growth factor. An introduced marker gene may confer antibiotic resistance, or encode an essential growth factor or enzyme, thereby permitting growth on selective media when expressed in the transformed host. Selection of a transformed host can also occur when the expressed marker protein can be detected, either directly or indirectly. The marker protein may be expressed alone or as a fusion to another protein. Cells expressing the marker protein or tag can be selected, for example, visually, or by techniques such as fluorescence-activated cell sorting or panning using antibodies.

Regardless of the selected host or expression construct, multiple transformants must be screened to obtain a strain or line displaying the desired expression level, regulation and pattern, as different independent transformation events result in different levels and patterns of expression (Jones et al., EMBO J., 4:2411-2418 (1985); De Almeida et al., Mol. Gen. Genetics, 218:78-86 (1989)). Such screening may be accomplished by Southern analysis of DNA blots (Southern, J. Mol. Biol., 98:503 (1975)), Northern analysis of mRNA expression (Kroczek, J. Chromatogr. Biomed. Appl., 618(1-2):133-145 (1993)), and Western and/or Elisa analyses of protein expression or phenotypic analysis. Alternately, by simply quantifying the amount of the non-native product of interest produced in the transgenic microorganism in which the expression level of G6PDH and 6PGL have been manipulated, and comparing this to the amount of non-native product of interest produced in the transgenic microorganism in which the expression level of G6PDH and 6PGL have not been manipulated, one will readily be able to determine if coordinately regulated over-expression of G6PDH and 6PGL has been achieved based on whether an increased amount of the non-native product of interest is observed in the cell. The particular assay will be determined based on the product of interest that is synthesized.

The transgenic microorganism is grown under conditions that optimize production of the at least one non-native product of interest. In general, media conditions may be optimized by modifying the type and amount of carbon source, the type and amount of nitrogen source, the carbon-to-nitrogen ratio, the amount of different mineral ions, the oxygen level, growth temperature, pH, length of the biomass production phase, length of the oil accumulation phase and the time and method of cell harvest. For example, the oleaginous yeast Yarrowia lipolytica is generally grown in a complex medium such as yeast extract-peptone-dextrose broth [“YPD”], a defined minimal media, or a defined minimal media that lacks a component necessary for growth and forces selection of the desired expression cassettes (e.g., Yeast Nitrogen Base (DIFCO Laboratories, Detroit, Mich.)).

Fermentation media for the methods and transgenic organisms described herein must contain a suitable carbon source such as taught in U.S. Pat. No. 7,238,482 and U.S. Pat. Pub. No. 2009-0325265-A1. Suitable sources of carbon encompass a wide variety of sources, with sugars (e.g., glucose), fructose, glycerol and/or fatty acids being preferred. Most preferred is glucose, sucrose, invert sucrose, fructose and/or fatty acids containing between 10-22 carbons. For example, the fermentable carbon source can be selected from the group consisting of invert sucrose (i.e., a mixture comprising equal parts of fructose and glucose resulting from the hydrolysis of sucrose), glucose, fructose and combinations of these, provided that glucose is used in combination with invert sucrose and/or fructose.

Nitrogen may be supplied from an inorganic (e.g., (NH₄)₂SO₄) or organic (e.g., urea or glutamate) source. In addition to appropriate carbon and nitrogen sources, the fermentation media must also contain suitable minerals, salts, cofactors, buffers, vitamins and other components known to those skilled in the art suitable for the growth of the oleaginous host and promotion of the enzymatic pathways necessary for production of the non-native product of interest. Preferred growth media are common commercially prepared media, such as Yeast Nitrogen Base (DIFCO Laboratories, Detroit, Mich.). Other defined or synthetic growth media may also be used and the appropriate medium for growth of the transformant host cells will be known by one skilled in the art of microbiology or fermentation science. A suitable pH range for the fermentation is typically between about pH 4.0 to pH 8.0, wherein pH 5.5 to pH 7.5 is preferred as the range for the initial growth conditions. The fermentation may be conducted under aerobic or anaerobic conditions, wherein microaerobic conditions are preferred.

One of skill in the art will also be familiar with the appropriate means to culture the transgenic microorganism, based on the particular product of interest that is being produced. For example, accumulation of high levels of PUFAs in oleaginous yeast cells typically requires a two-stage process, since the metabolic state must be “balanced” between growth and synthesis/storage of fats. Thus, most preferably, a two-stage fermentation process is necessary for the production of PUFAs in oleaginous yeast (e.g., Yarrowia lipolytica). This approach is described in U.S. Pat. No. 7,238,482, as are various suitable fermentation process designs (i.e., batch, fed-batch and continuous) and considerations during growth.

### EXAMPLES

The present invention is further defined in the following Examples. It should be understood that these Examples, while indicating preferred aspects of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

Unless otherwise specified, all referenced United States patents and patent applications are hereby incorporated by reference.

### General Methods

Standard recombinant DNA and molecular cloning techniques used in the Examples are well known in the art and are described by: 1) Sambrook, J., Fritsch, E. F. and Maniatis, T., Molecular Cloning: A Laboratory Manual; Cold Spring Harbor Laboratory: Cold Spring Harbor, N.Y. (1989) (Maniatis); 2) T. J. Silhavy, M. L. Bennan, and L. W. Enquist, Experiments with Gene Fusions; Cold Spring Harbor Laboratory: Cold Spring Harbor, N.Y. (1984); and, 3) Ausubel, F. M. et al., Current Protocols in Molecular Biology, published by Greene Publishing Assoc. and Wiley-Interscience, Hoboken, N.J. (1987).

Materials and methods suitable for the maintenance and growth of microbial cultures are well known in the art. Techniques suitable for use in the following examples may be found as set out in Manual of Methods for General Bacteriology (Phillipp Gerhardt, R. G. E. Murray, Ralph N. Costilow, Eugene W. Nester, Willis A. Wood, Noel R. Krieg and G. Briggs Phillips, Eds), American Society for Microbiology: Washington, D.C. (1994)); or by Thomas D. Brock in Biotechnology: A Textbook of Industrial Microbiology, 2^{nd} ed., Sinauer Associates Sunderland, Mass. (1989). All reagents, restriction enzymes and materials used for the growth and maintenance of microbial cells were obtained from Aldrich Chemicals (Milwaukee, Wis.), DIFCO Laboratories (Detroit, Mich.), New England Biolabs, Inc. (Beverly, Mass.), GIBCO/BRL (Gaithersburg, Md.), or Sigma Chemical Company (St. Louis, Mo.), unless otherwise specified. E. coli strains were typically grown at 37° C. on Luria Bertani [“LB”] plates.

Unless otherwise specified, PCR amplifications were carried out in a 50 μl total volume, comprising: PCR buffer (containing 10 mM KCl, 10 mM (NH₄)₂SO₄, 20 mM Tris-HCl (pH 8.75), 2 mM MgSO₄, 0.1% Triton X-100), 100 μg/mL BSA (final concentration), 200 μM each deoxyribonucleotide triphosphate, 10 pmole of each primer, 1 μl of Pfu DNA polymerase (Stratagene, San Diego, Calif.) and 20-100 ng of template DNA in 1 μl volume. Amplification was carried out as follows: initial denaturation at 95° C. for 1 min, followed by 30 cycles of denaturation at 95° C. for 30 sec, annealing at 55° C. for 1 min, and elongation at 72° C. for 1 min. A final elongation cycle at 72° C. for 10 min was carried out, followed by reaction termination at 4° C.

General molecular cloning was performed according to standard methods (Sambrook et al., supra). DNA sequence was generated on an ABI Automatic sequencer using dye terminator technology (U.S. Pat. No. 5,366,860; EP 272,007) using a combination of vector and insert-specific primers. Sequence editing was performed in Sequencher (Gene Codes Corporation, Ann Arbor, Mich.). All sequences represent coverage at least two times in both directions. Unless otherwise indicated herein comparisons of genetic sequences were accomplished using DNASTAR software (DNASTAR Inc., Madison, Wis.). The meaning of abbreviations is as follows: “sec” means second(s), “min” means minute(s), “h” means hour(s), “d” means day(s), “μL” means microliter(s), “mL” means milliliter(s), “L” means liter(s), “μM” means micromolar, “mM” means millimolar, “M” means molar, “mmol” means millimole(s), “μmole” mean micromole(s), “g” means gram(s), “μg” means microgram(s), “ng” means nanogram(s), “U” means unit(s), “bp” means base pair(s) and “kB” means kilobase(s).

### Nomenclature For Expression Cassettes

The structure of an expression cassette is represented by a simple notation system of “X::Y::Z”, wherein X describes the promoter fragment, Y describes the gene fragment, and Z describes the terminator fragment, which are all operably linked to one another.

Transformation And Cultivation Of Yarrowia lipolytica

Yarrowia lipolytica strain ATCC #20362 was purchased from the American Type Culture Collection (Rockville, Md.). Yarrowia lipolytica strains were routinely grown at 28-30° C. in several media, according to the recipes shown below.
High Glucose Media [“HGM”] (per liter): 80 glucose, 2.58 g KH₂PO₄ and
   5.36 g K₂HPO₄, pH 7.5 (do not need to adjust).
Synthetic Dextrose Media [“SD”] (per liter): 6.7 g Yeast Nitrogen base with ammonium sulfate and without amino acids, and 20 g glucose.
Fermentation medium [“FM”] (per liter): 6.70 g/L Yeast nitrogen base with ammonium sulfate and without amino acids, 6.00 g KH₂PO₄, 2.00 g K₂HPO₄, 1.50 g MgSO₄*7H₂O, 1.5 mg/L thiamine-HCl, 20 g glucose, and 5.00 g Yeast extract (BBL).

Transformation of Y. lipolytica was performed as described in U.S. Pat. Appl. Pub. No. 2009-0093543-A1, hereby incorporated herein by reference.

Generation Of Yarrowia lipolytica Strain Y4305U

Strain Y4305U, producing EPA relative to the total lipids via expression of a Δ9 elongase/Δ8 desaturase pathway, was generated as described in the General Methods of U.S. Pat. App. Pub. No. 2008-0254191, hereby incorporated herein by reference. Briefly, strain Y4305U was derived from Yarrowia lipolytica ATCC #20362 via construction of strain Y2224 (a 5-fluoroorotic acid [“FOA”] resistant mutant from an autonomous mutation of the Ura3 gene of wildtype Yarrowia strain ATCC #20362), strain Y4001 (producing 17% EDA with a Leu-phenotype), strain Y4001U1 (Leu- and Ura-), strain Y4036 (producing 18% DGLA with a Leu-phenotype), strain Y4036U (Leu- and Ura-), strain Y4070 (producing 12% ARA with a Ura-phenotype), strain Y4086 (producing 14% EPA), strain Y4086U1 (Ura3-), strain Y4128 (producing 37% EPA; deposited with the American Type Culture Collection on Aug. 23, 2007, bearing the designation ATCC PTA-8614), strain Y4128U3 (Ura-), strain Y4217 (producing 42% EPA), strain Y4217U2 (Ura-), strain Y4259 (producing 46.5% EPA), strain Y4259U2 (Ura-) and strain Y4305 (producing 53.2% EPA relative to the total TFAs).

The complete lipid profile of strain Y4305 was as follows: 16:0 (2.8%), 16:1 (0.7%), 18:0 (1.3%), 18:1 (4.9%), 18:2 (17.6%), ALA (2.3%), EDA (3.4%), DGLA (2.0%), ARA (0.6%), ETA (1.7%), and EPA (53.2%). The total lipid % dry cell weight [“DCW”] was 27.5.

The final genotype of strain Y4305 with respect to wild type Yarrowia lipolytica ATCC #20362 was SCP2-(YALI0E01298g), YALI0C18711g-, Pex10-, YALI0F24167g-, unknown 1-, unknown 3-, unknown 8-, GPD::FmD12::Pex20, YAT1::FmD12::OCT, GPM/FBAIN::FmD12S::OCT, EXP1::FmD12S::Aco, YAT1::FmD12S::Lip2, YAT1::ME3S::Pex16, EXP1::ME3S::Pex20 (3 copies), GPAT::EgD9e::Lip2, EXP1::EgD9eS::Lip1, FBAINm::EgD9eS::Lip2, FBA::EgD9eS::Pex20, GPD::EgD9eS::Lip2, YAT1::EgD9eS::Lip2, YAT1::E389D9eS::OCT, FBAINm::EgD8M::Pex20, FBAIN::EgD8M::Lip1 (2 copies), EXP1::EgD8M::Pex16, GPDIN::EgD8M::Lip1, YAT1::EgD8M::Aco, FBAIN::EgD5::Aco, EXP1::EgD5S::Pex20, YAT1::EgD5S::Aco, EXP1::EgD5S::ACO, YAT1::RD5S::OCT, YAT1::PaD17S::Lip1, EXP1::PaD17::Pex16, FBAINm::PaD17::Aco, YAT1::YICPT1::ACO, GPD::YICPT1::ACO (wherein FmD12 is a Fusarium moniliforme Δ12 desaturase gene [U.S. Pat. No. 7,504,259]; FmD12S is a codon-optimized Δ12 desaturase gene, derived from Fusarium moniliforme [U.S. Pat. No. 7,504,259]; ME3S is a codon-optimized C_{16/18} elongase gene, derived from Mortierella alpina [U.S. Pat. No. 7,470,532]; EgD9e is a Euglena gracilis Δ9 elongase gene [U.S. Pat. No. 7,645,604]; EgD9eS is a codon-optimized Δ9 elongase gene, derived from Euglena gracilis [U.S. Pat. No. 7,645,604]; E389D9eS is a codon-optimized Δ9 elongase gene, derived from Eutreptiella sp. CCMP389 [U.S. Pat. No. 7,645,604]; EgD8M is a synthetic mutant Δ8 desaturase [U.S. Pat. No. 7,709,239], derived from Euglena gracilis [U.S. Pat. No. 7,256,033]; EgD5 is a Euglena gracilis Δ5 desaturase [U.S. Pat. No. 7,678,560]; EgD5S is a codon-optimized Δ5 desaturase gene, derived from Euglena gracilis [U.S. Pat. No. 7,678,560]; RD5S is a codon-optimized Δ5 desaturase, derived from Peridinium sp. CCMP626 [U.S. Pat. No. 7,695,950]; PaD17 is a Pythium aphanidermatum Δ17 desaturase [U.S. Pat. No. 7,556,949]; PaD17S is a codon-optimized Δ17 desaturase, derived from Pythium aphanidermatum [U.S. Pat. No. 7,556,949]; and, YICPT1 is a Yarrowia lipolytica diacylglycerol cholinephosphotransferase gene [Int'l. App. Pub. No. WO 2006/052870]).

The Ura3 gene was subsequently disrupted in strain Y4305 (as described in the General Methods of U.S. Pat. App. Pub. No. 2008-0254191), such that a Ura3 mutant gene was integrated into the Ura3 gene of strain Y4305. Following selection of the transformants and analysis of the FAMEs, transformants #1, #6 and #7 were determined to produce 37.6%, 37.3% and 36.5% EPA of total lipids, respectively, when grown on MM+5-FOA plates. These three strains were designated as strains Y4305U1, Y4305U2 and Y4305U3, respectively, and are collectively identified as strain Y4305U.

Fatty Acid Analysis of Yarrowia lipolytica

For fatty acid analysis, cells were collected by centrifugation and lipids were extracted as described in Bligh, E. G. & Dyer, W. J. (Can. J. Biochem. Physiol., 37:911-917 (1959)). Fatty acid methyl esters [“FAMEs”] were prepared by transesterification of the lipid extract with sodium methoxide (Roughan, G., and Nishida I., Arch Biochem Biophys., 276(1):38-46 (1990)) and subsequently analyzed with a Hewlett-Packard 6890 GC fitted with a 30-m×0.25 mm (i.d.) HP-INNOWAX (Hewlett-Packard) column. The oven temperature was from 170° C. (25 min hold) to 185° C. at 3.5° C./min.

For direct base transesterification, Yarrowia culture (3 mL) was harvested, washed once in distilled water, and dried under vacuum in a Speed-Vac for 5-10 min. Sodium methoxide (100 μl of 1%) was added to the sample, and then the sample was vortexed and rocked for 20 min. After adding 3 drops of 1 M NaCl and 400 μl hexane, the sample was vortexed and spun. The upper layer was removed and analyzed by GC as described above.

### Yarrowia Genes Encoding G6PDH, 6PGL And 6PGDH

The Yarrowia lipolytica gene encoding glucose-6-phosphate dehydrogenase [“G6PDH”] is set forth herein as SEQ ID NO:1 and corresponds to GenBank Accession No. XM_{—}504275. Annotated therein as Yarrowia lipolytica ORF YALI0E22649p, the 1497 bp sequence is “similar to uniprot|P11412 Saccharomyces cerevisiae YNL241c ZWF1 glucose-6-phosphate dehydrogenase”.

Additionally, using the 498 amino acid protein sequence encoding the Yarrowia lipolytica G6PDH (SEQ ID NO:2), National Center for Biotechnology Information [“NCBI”] BLASTP 2.2.22+ (Basic Local Alignment Search Tool; Altschul, S. F., et al., Nucleic Acids Res., 25:3389-3402 (1997); Altschul, S. F., et al., FEBS J., 272:5101-5109 (2005)) searches were conducted to identify sequences having similarity within the BLAST “nr” database (comprising all non-redundant GenBank CDS translations, the Protein Data Bank [“PDB”] protein sequence database, the SWISS-PROT protein sequence database, the Protein Information Resource [“PIR”] protein sequence database and the Protein Research Foundation [“PRF”] protein sequence database, excluding environmental samples from whole genome shotgun [“WGS”] projects).

The results of the BLASTP comparison summarizing the sequence to which SEQ ID NO:2 has the most similarity are reported according to the % identity, % similarity and Expectation value. “% Identity” is defined as the percentage of amino acids that are identical between the two proteins. “% Similarity” is defined as the percentage of amino acids that are identical or conserved between the two proteins. “Expectation value” estimates the statistical significance of the match, specifying the number of matches, with a given score, that are expected in a search of a database of this size absolutely by chance.

A large number of proteins were identified as sharing significant similarity to the Yarrowia lipolytica G6PDH (SEQ ID NO:2). Table 3 provides a partial summary of those hits having annotation that specifically identified the protein as a “glucose-6-phosphate dehydrogenase”, although this should not be considered as limiting to the disclosure herein. The proteins in Table 3 had an e-value greater than 2e-132 with SEQ ID NO:2.

TABLE 3
Examples Of Some Publicly Available Genes Encoding Glucose-6-
Phosphate Dehydrogenase

| | | Query | |
|---|---|---|---|
| Accession | Description | coverage | E value |
| XP_365081.2 | glucose-6-phosphate 1-dehydrogenase | 97% | 0.0 |
| | [Magnaporthe grisea 70-15] | | |
| XP_381455.1 | Glucose-6-phosphate 1-dehydrogenase | 97% | 0.0 |
| | (G6PD) [Gibberella zeae PH-1] | | |
| XP_001553624.1 | glucose-6-phosphate 1-dehydrogenase | 97% | 0.0 |
| | [Botryotinia fuckeliana B05.10] | | |
| XP_660585.1 | Glucose-6-phosphate 1-dehydrogenase | 98% | 0.0 |
| | (G6PD) [Aspergillus nidulans FGSC A4] | | |
| EEH10762.1 | glucose-6-phosphate 1-dehydrogenase | 98% | 0.0 |
| | [Ajellomyces capsulatus G186AR] | | |
| XP_002373576.1 | glucose-6-phosphate 1-dehydrogenase | 97% | 0.0 |
| | [Aspergillus flavus NRRL3357] | | |
| XP_002627278.1 | glucose-6-phosphate dehydrogenase | 97% | 0.0 |
| | [Ajellomyces dermatitidis SLH14081] | | |
| XP_001400342.1, | glucose-6-phosphate 1-dehydrogenase | 98% | 0.0 |
| CAA54840.1 | [Aspergillus niger] | | |
| EEQ33299.1 | glucose-6-phosphate 1-dehydrogenase | 97% | 0.0 |
| | [Microsporum canis CBS 113480] | | |
| XP_002153443.1, | glucose-6-phosphate 1-dehydrogenase | 97% | 1e−180 |
| XP_002153442.1 | [Penicillium marneffei ATCC 18224] | | |
| XP_001208988.1 | glucose-6-phosphate 1-dehydrogenase | 99% | 1e−180 |
| | [Aspergillus terreus NIH2624] | | |
| XP_001931341.1 | glucose-6-phosphate 1-dehydrogenase | 97% | 3e−180 |
| | [Pyrenophora tritici-repentis Pt-1C-BFP] | | |
| XP_001240498.1 | glucose-6-phosphate 1-dehydrogenase | 97% | 3e−180 |
| | [Coccidioides immitis RS] | | |
| XP_001263592.1 | glucose-6-phosphate 1-dehydrogenase | 97% | 8e−180 |
| | [Neosartorya fischeri NRRL 181] | | |
| EEH48116.1 | glucose-6-phosphate 1-dehydrogenase | 97% | 2e−179 |
| | [Paracoccidioides brasiliensis Pb18] | | |
| EEH37712.1 | glucose-6-phosphate 1-dehydrogenase | 97% | 2e−179 |
| | [Paracoccidioides brasiliensis Pb01] | | |
| XP_002487987.1, | glucose-6-phosphate 1-dehydrogenase | 97% | 2e−179 |
| XP_002487986.1 | [Talaromyces stipitatus ATCC 10500] | | |
| XP_001270867.1 | glucose-6-phosphate 1-dehydrogenase | 97% | 2e−179 |
| | [Aspergillus clavatus NRRL 1] | | |
| XP_754767.1 | glucose-6-phosphate 1-dehydrogenase | 97% | 1e−178 |
| | [Aspergillus fumigatus Af293] | | |
| XP_958320.2 | glucose-6-phosphate 1-dehydrogenase | 99% | 1e−178 |
| | [Neurospora crassa OR74A] | | |
| XP_001220826.1 | glucose-6-phosphate 1-dehydrogenase | 97% | 8e−177 |
| | [Chaetomium globosum CBS 148.51] | | |
| XP_001540489.1 | glucose-6-phosphate 1-dehydrogenase | 98% | 2e−175 |
| | [Ajellomyces capsulatus NAm1] | | |
| EEQ88494.1 | glucose-6-phosphate dehydrogenase | 91% | 9e−175 |
| | [Ajellomyces dermatitidis ER-3] | | |
| XP_001386049.2 | Glucose-6-phosphate 1-dehydrogenase | 97% | 5e−174 |
| | [Pichia stipitis CBS 6054] | | |
| XP_002582851.1 | glucose-6-phosphate dehydrogenase | 97% | 3e−173 |
| | [Uncinocarpus reesii 1704] | | |
| XP_002548953.1 | glucose-6-phosphate 1-dehydrogenase | 97% | 1e−172 |
| | [Candida tropicalis MYA-3404] | | |
| XP_002491203.1 | Glucose-6-phosphate dehydrogenase | 98% | 2e−172 |
| | (G6PD), [Pichia pastoris GS115] | | |
| ACJ12748.1 | glucose-6-phosphate dehydrogenase | 97% | 2e−171 |
| | [Candida tropicalis] | | |
| EEH19267.1 | glucose-6-phosphate 1-dehydrogenase | 97% | 3e−171 |
| | [Paracoccidioides brasiliensis Pb03] | | |
| XP_002417491.1 | glucose-6-phosphate 1-dehydrogenase, | 98% | 2e−170 |
| | putative [Candida dubliniensis CD36] | | |
| P11410.2 | glucose-6-phosphate dehydrogenase [Pichia | 97% | 2e−170 |
| | jadinii] | | |
| XP_723251.1 | likely glucose-6-phosphate dehydrogenase | 97% | 7e−170 |
| | [Candida albicans SC5314] | | |
| XP_723440.1 | likely glucose-6-phosphate dehydrogenase | 97% | 2e−169 |
| | [Candida albicans SC5314]] | | |
| XP_001527991.1 | glucose-6-phosphate 1-dehydrogenase | 98% | 1e−167 |
| | [Lodderomyces elongisporus NRRL YB-4239] | | |
| XP_572045.1 | glucose-6-phosphate 1-dehydrogenase | 99% | 2e−167 |
| | [Cryptococcus neoformans var. neoformans | | |
| | JEC21] | | |
| XP_453944.1 | Glucose-6-phosphate 1-dehydrogenase | 97% | 1e−165 |
| | (G6PD) [Kluyveromyces lactis] | | |
| EDN62584.1 | glucose-6-phosphate dehydrogenase | 98% | 2e−165 |
| | [Saccharomyces cerevisiae YJM789] | | |
| EEU07329.1 | Zwf1p [Saccharomyces cerevisiae JAY291] | 98% | 3e−165 |
| CAY82368.1 | Zwf1p [Saccharomyces cerevisiae EC1118] | 98% | 4e−165 |
| NP_014158.1 | Glucose-6-phosphate 1-dehydrogenase | 98% | 2e−164 |
| | (G6PD) [Saccharomyces cerevisiae] | | |
| AAT93017.1 | YNL241C [Saccharomyces cerevisiae] | 98% | 3e−164 |
| AAA34619.1 | glucose-6-phosphate dehydrogenase (ZWF1) | 98% | 1e−163 |
| | (EC 1.1.1.49) [Saccharomyces cerevisiae] | | |
| XP_001876685.1 | glucose-6-P dehydrogenase [Laccaria bicolor | 100% | 2e−161 |
| | S238N-H82] | | |
| CAQ43421.1 | Glucose-6-phosphate 1-dehydrogenase | 98% | 1e−158 |
| | [Zygosaccharomyces rouxii] | | |
| EEY18838.1 | glucose-6-phosphate 1-dehydrogenase | 87% | 3e−158 |
| | [Verticillium albo-atrum VaMs.102] | | |
| XP_002173507.1 | glucose-6-phosphate 1-dehydrogenase | 97% | 7e−153 |
| | [Schizosaccharomyces japonicus yFS275] | | |
| NP_593344.2 | glucose-6-phosphate 1-dehydrogenase | 96% | 7e−147 |
| | (predicted) [Schizosaccharomyces pombe] | | |
| ABD72519.1 | glucose 6-phosphate dehydrogenase | 94% | 1e−138 |
| | [Trypanosoma cruzi] | | |
| XP_820060.1 | glucose-6-phosphate 1-dehydrogenase | 95% | 2e−137 |
| | [Trypanosoma cruzi strain CL Brener] | | |
| ABD72518.1 | glucose 6-phosphate dehydrogenase | 95% | 3e−137 |
| | [Trypanosoma cruzi] | | |
| NP_198892.1 | glucose-6-phosphate dehydrogenase | 98% | 2e−136 |
| | (G6PD6) [Arabidopsis thaliana] | | |
| EFA81744.1 | glucose 6-phosphate-1-dehydrogenase | 97% | 4e−136 |
| | [Polysphondylium pallidum PN500] | | |
| CAB52675.1 | glucose-6-phosphate 1-dehydrogenase | 98% | 5e−136 |
| | [Arabidopsis thaliana] | | |
| ABF20372.1 | glucose-6-phosphate dehydrogenase | 96% | 7e−136 |
| | [Leishmania gerbilli] | | |
| ABF20357.1 | glucose-6-phosphate dehydrogenase | 94% | 2e−135 |
| | [Leishmania donovani] | | |
| XP_644436.1 | glucose 6-phosphate-1-dehydrogenase | 98% | 3e−135 |
| | [Dictyostelium discoideum AX4] | | |
| ABF20355.1, | glucose-6-phosphate dehydrogenase | 94% | 3e−135 |
| ABF20345.1, | [Leishmania infantum] | | |
| XP_001468395.1 | | | |
| XP_001686097.1 | glucose-6-phosphate dehydrogenase | 96% | 6e−135 |
| | [Leishmania major] | | |
| ABF20370.1 | glucose-6-phosphate dehydrogenase | 94% | 8e−135 |
| | [Leishmania infantum] | | |
| XP_822502.1 | glucose-6-phosphate 1-dehydrogenase | 95% | 2e−134 |
| | [Trypanosoma brucei TREU927] | | |
| CAC07816.1 | glucose-6-phosphate 1-dehydrogenase | 95% | 3e−134 |
| | [Trypanosoma brucei] | | |
| CBH15225.1 | glucose-6-phosphate 1-dehydrogenase, | 95% | 3e−134 |
| | putative [Trypanosoma brucei gambiense | | |
| | DAL972] | | |
| XP_002126015.1 | PREDICTED: similar to glucose-6-phosphate | 97% | 4e−134 |
| | dehydrogenase isoform b (predicted) [Ciona | | |
| | intestinalis] | | |
| AAO37825.1 | glucose-6-phosphate dehydrogenase | 94% | 5e−134 |
| | [Leishmania mexicana] | | |
| BAB96757.1 | glucose-6-phosphate dehydrogenase 1 | 96% | 6e−134 |
| | [Chlorella vulgaris] | | |
| XP_001848152.1 | glucose-6-phosphate 1-dehydrogenase | 96% | 1e−133 |
| | [Culex quinquefasciatus] | | |
| AAM64228.1 | glucose-6-phosphate dehydrogenase | 96% | 2e−133 |
| | [Leishmania amazonensis] | | |
| ABU25160.1 | glucose-6-phosphate dehydrogenase | 96% | 7e−133 |
| | [Leishmania panamensis] | | |
| ABU25155.1 | glucose-6-phosphate dehydrogenase | 96% | 9e−133 |
| | [Leishmania braziliensis] | | |
| ABU25158.1, | glucose-6-phosphate dehydrogenase | 96% | 2e−132 |
| XP_001564303.1 | [Leishmania braziliensis] | | |
| AAM64230.1 | glucose-6-phosphate dehydrogenase | 96% | 2e−132 |
| | [Leishmania guyanensis] | | |

It should be noted that G6PDH is found in all organisms and cell types where it has been sought and considerable sequence conservation is observed. Nogae, I. and M. Johnston (Gene, 96:161-169 (1990)), who first isolated and characterized the ZWF1 gene of Saccharomyces cerevisiae encoding G6PDH, noted that the encoded protein was about 60% similar to G6PDH sequences from Drosophila, human and rat enzymes.

The Yarrowia lipolytica gene encoding 6-phosphogluconolactonase [“6PGL”] is set forth herein as SEQ ID NO:3 and corresponds to GenBank Accession No. XM_{—}503830. Annotated therein as Yarrowia lipolytica ORF YALI0E11671p, the 747 bp sequence is “similar to uniprot|P38858 Saccharomyces cerevisiae YHR163w SOL3 possible 6-phosphogluconolactonase”.

The 248 amino acid protein sequence encoding the Yarrowia lipolytica 6PGL (SEQ ID NO:4) was used as the query in a NCBI BLASTP 2.2.22+ search against the “nr” database in a manner similar to that as described above for the Y. lipolytica G6PDH protein. A large number of proteins were identified as sharing significant similarity to SEQ ID NO:4. Table 4 provides a partial summary of those hits having annotation that specifically identified the protein as a “6-phosphogluconolactonase”, although this should not be considered as limiting to the disclosure herein. The proteins in Table 4 had an e-value greater than 1e-40 with SEQ ID NO:4.

TABLE 4
Examples Of Some Publicly Available Genes Encoding 6-
Phosphogluconolactonase

| | | Query | |
|---|---|---|---|
| Accession | Description | coverage | E value |
| XP_001382491.2 | 6-phosphogluconolactonase-like protein [Pichia | 97% | 2e−60 |
| | stipitis CBS 6054] | | |
| XP_002422184.1 | 6-phosphogluconolactonase, putative [Candida | 97% | 2e−58 |
| | dubliniensis CD36] | | |
| XP_711795.1 | potential 6-phosphogluconolactonase [Candida | 97% | 3e−58 |
| | albicans SC5314] | | |
| XP_002493372.1 | 6-phosphogluconolactonase [Pichia pastoris | 99% | 4e−58 |
| | GS115] | | |
| XP_002372956.1 | 6-phosphogluconolactonase, putative [Aspergillus | 99% | 1e−55 |
| | flavus NRRL3357] | | |
| CBF89810.1 | TPA: 6-phosphogluconolactonase, putative | 99% | 5e−55 |
| | [Aspergillus nidulans FGSC A4] | | |
| XP_001481696.1 | 6-phosphogluconolactonase [Aspergillus fumigatus | 99% | 4e−54 |
| | Af293] | | |
| EDP55639.1 | 6-phosphogluconolactonase, putative [Aspergillus | 99% | 4e−54 |
| | fumigatus A1163] | | |
| XP_001269838.1 | 6-phosphogluconolactonase [Aspergillus clavatus | 99% | 1e−53 |
| | NRRL 1] | | |
| EEH34572.1 | 6-phosphogluconolactonase [Paracoccidioides | 98% | 1e−53 |
| | brasiliensis Pb01] | | |
| EEH42951.1 | 6-phosphogluconolactonase [Paracoccidioides | 98% | 2e−53 |
| | brasiliensis Pb18] | | |
| XP_001265354.1 | 6-phosphogluconolactonase, putative [Neosartorya | 99% | 3e−53 |
| | fischeri NRRL 181] | | |
| EEH16106.1 | 6-phosphogluconolactonase [Paracoccidioides | 98% | 7e−53 |
| | brasiliensis Pb03] | | |
| EEQ33166.1 | 6-phosphogluconolactonase [Microsporum canis | 91% | 2e−52 |
| | CBS 113480] | | |
| XP_002624608.1 | 6-phosphogluconolactonase [Ajellomyces | 97% | 1e−51 |
| | dermatitidis SLH14081] | | |
| EEQ86414.1 | 6-phosphogluconolactonase [Ajellomyces | 97% | 1e−51 |
| | dermatitidis ER-3] | | |
| EEH11202.1 | 6-phosphogluconolactonase [Ajellomyces | 94% | 6e−51 |
| | capsulatus G186AR] | | |
| XP_002149918.1 | 6-phosphogluconolactonase, putative [Penicillium | 99% | 1e−50 |
| | marneffei ATCC 18224] | | |
| XP_002484346.1 | 6-phosphogluconolactonase, putative | 89% | 2e−50 |
| | [Talaromyces stipitatus ATCC 10500] | | |
| XP_571054.1 | 6-phosphogluconolactonase [Cryptococcus | 99% | 2e−50 |
| | neoformans var. neoformans JEC21] | | |
| NP_012033.2 | 6-phosphogluconolactonase (6PGL), catalyzes the | 88% | 6e−50 |
| | 2^{nd} step of the pentose phosphate pathway; | | |
| | homologous to Sol2p and Sol1p [Saccharomyces | | |
| | cerevisiae] | | |
| AAB68008.1 | Sol3p [Saccharomyces cerevisiae] | 88% | 6e−50 |
| EER29331.1 | 6-phosphogluconolactonase, putative [Coccidioides | 97% | 2e−49 |
| | posadasii C735 delta SOWgp] | | |
| EEY55014.1 | 6-phosphogluconolactonase, putative | 91% | 1e−48 |
| | [Phytophthora infestans T30-4] | | |
| EER43253.1 | 6-phosphogluconolactonase [Ajellomyces | 94% | 2e−48 |
| | capsulatus H143] | | |
| NP_587920.1 | 6-phosphogluconolactonase (predicted) | 98% | 5e−48 |
| | [Schizosaccharomyces pombe 972h-] | | |
| NP_079672.1 | 6-phosphogluconolactonase [Mus musculus] | 96% | 5e−47 |
| NP_001099536.1 | 6-phosphogluconolactonase [Rattus norvegicus] | 96% | 2e−46 |
| XP_001873891.1 | 6-phosphogluconolactonase [Laccaria bicolor | 93% | 1e−45 |
| | S238N-H82] | | |
| NP_014432.1 | 6-phosphogluconolactonase-like protein 1; Sol1p | 84% | 5e−45 |
| | [Saccharomyces cerevisiae] | | |
| XP_002173062.1 | 6-phosphogluconolactonase | 97% | 6e−45 |
| | [Schizosaccharomyces japonicus yFS275] | | |
| EEY22743.1 | 6-phosphogluconolactonase [Verticillium albo- | 75% | 7e−45 |
| | atrum VaMs.102] | | |
| XP_002496785.1 | Probable 6-phosphogluconolactonase 1 and | 85% | 1e−43 |
| | Probable 6-phosphogluconolactonase 2 | | |
| | [Zygosaccharomyces rouxii] | | |
| XP_001173626.1 | PREDICTED: 6-phosphogluconolactonase isoform | 95% | 1e−43 |
| | 3 [Pan troglodytes] | | |
| NP_009999.2 | 6-phosphogluconolactonase-like protein 2; Sol2p | 85% | 2e−43 |
| | [Saccharomyces cerevisiae] | | |
| NP_036220.1 | 6-phosphogluconolactonase (6PGL) [Homo | 92% | 3e−43 |
| | sapiens] | | |
| XP_001517951.1 | PREDICTED: similar to 6- | 96% | 2e−42 |
| | phosphogluconolactonase [Ornithorhynchus | | |
| | anatinus] | | |
| XP_001937609.1 | 6-phosphogluconolactonase [Pyrenophora tritici- | 90% | 3e−42 |
| | repentis Pt-1C-BFP] | | |
| ACO09969.1 | 6-phosphogluconolactonase [Osmerus mordax] | 88% | 3e−42 |
| XP_852582.1 | PREDICTED: similar to 6- | 96% | 3e−42 |
| | phosphogluconolactonase [Canis familiaris] | | |
| XP_570172.1 | 6-phosphogluconolactonase [Cryptococcus | 91% | 1e−41 |
| | neoformans var. neoformans JEC21] | | |
| XP_001648196.1 | 6-phosphogluconolactonase [Aedes aegypti] | 93% | 5e−41 |
| XP_001368707.1 | PREDICTED: similar to 6- | 85% | 7e−41 |
| | phosphogluconolactonase [Monodelphis | | |
| | domestica] | | |
| NP_001140068.1 | 6-phosphogluconolactonase [Salmo salar] | 92% | 1e−40 |

Similarly, the Yarrowia lipolytica gene encoding 6-phosphogluconate dehydrogenase [“6PGDH”] is set forth herein as SEQ ID NO:5 and corresponds to GenBank Accession No. XM_{—}500938. Annotated therein as Yarrowia lipolytica ORF YALI0B15598p, the 1470 bp sequence is “highly similar to uniprot|P38720 Saccharomyces cerevisiae YHR183w GND1 6-phosphogluconate dehydrogenase”.

The 489 amino acid protein sequence encoding the Yarrowia lipolytica 6PGDH (SEQ ID NO:6) was used as the query in a NCBI BLASTP 2.2.22+ search against the “nr” database in a manner similar to that as described above for the Y. lipolytica G6PDH and 6PGL proteins. A large number of proteins were identified as sharing significant similarity to SEQ ID NO:6. Table 5 provides a partial summary of those hits having annotation that specifically identified the protein as a “6-phosphogluconate dehydrogenase”, although this should not be considered as limiting to the disclosure herein. The proteins in Table 5 had an e-value greater than 0.0 with SEQ ID NO:6.

TABLE 5
Examples Of Some Publicly Available Genes Encoding 6-
Phosphogluconate Dehydrogenase

| | | Query | |
|---|---|---|---|
| Accession | Description | coverage | E value |
| XP_001525552.1 | 6-phosphogluconate dehydrogenase [Lodderomyces | 99% | 0.0 |
| | elongisporus NRRL YB-4239] | | |
| XP_002541572.1 | 6-phosphogluconate dehydrogenase | 98% | 0.0 |
| | (decarboxylating) [Uncinocarpus reesii 1704] | | |
| EDN61841.1 | 6-phosphogluconate dehydrogenase | 98% | 0.0 |
| | [Saccharomyces cerevisiae YJM789] | | |
| XP_001387191.1 | 6-phosphogluconate dehydrogenase [Pichia stipitis | 99% | 0.0 |
| | CBS 6054] | | |
| XP_002417924.1 | 6-phosphogluconate dehydrogenase, | 97% | 0.0 |
| | decarboxylating 1, putative [Candida dubliniensis | | |
| | CD36] | | |
| NP_011772.1 | 6-phosphogluconate dehydrogenase | 98% | 0.0 |
| | (decarboxylating) [Saccharomyces cerevisiae] | | |
| ACJ12750.1 | 6-phosphogluconate dehydrogenase [Candida | 99% | 0.0 |
| | tropicalis] | | |
| O13287.1 | 6-phosphogluconate dehydrogenase [Candida | 97% | 0.0 |
| | albicans] | | |
| EER23859.1 | 6-phosphogluconate dehydrogenase, putative | 98% | 0.0 |
| | [Coccidioides posadasii C735 delta SOWgp] | | |
| EDV10005.1 | 6-phosphogluconate dehydrogenase | 98% | 0.0 |
| | [Saccharomyces cerevisiae RM11-1a] | | |
| XP_001247382.1 | 6-phosphogluconate dehydrogenase, | 98% | 0.0 |
| | decarboxylating [Coccidioides immitis RS] | | |
| XP_002549363.1 | 6-phosphogluconate dehydrogenase [Candida | 100% | 0.0 |
| | tropicalis MYA-3404] | | |
| XP_002492495.1 | 6-phosphogluconate dehydrogenase | 98% | 0.0 |
| | (decarboxylating) [Pichia pastoris GS115] | | |
| XP_001257925.1 | 6-phosphogluconate dehydrogenase, | 97% | 0.0 |
| | decarboxylating [Neosartorya fischeri NRRL 181] | | |
| XP_001267994.1 | 6-phosphogluconate dehydrogenase, | 97% | 0.0 |
| | decarboxylating [Aspergillus clavatus NRRL 1] | | |
| XP_750696.1 | 6-phosphogluconate dehydrogenase Gnd1 | 97% | 0.0 |
| | [Aspergillus fumigatus Af293] | | |
| CAD80254.1 | 6-phosphogluconate dehydrogenase [Aspergillus | 98% | 0.0 |
| | niger] | | |
| EEQ35807.1 | 6-phosphogluconate dehydrogenase [Microsporum | 98% | 0.0 |
| | canis CBS 113480] | | |
| XP_002626217.1 | 6-phosphogluconate dehydrogenase [Ajellomyces | 97% | 0.0 |
| | dermatitidis SLH14081] | | |
| XP_002496776.1 | 6-phosphogluconate dehydrogenase, | 97% | 0.0 |
| | [Zygosaccharomyces rouxii] | | |
| XP_001819351.1 | 6-phosphogluconate dehydrogenase Gnd1, putative | 98% | 0.0 |
| | [Aspergillus flavus NRRL3357] | | |
| XP_002146717.1 | 6-phosphogluconate dehydrogenase Gnd1, putative | 98% | 0.0 |
| | [Penicillium marneffei ATCC 18224] | | |
| EEH47567.1 | 6-phosphogluconate dehydrogenase | 98% | 0.0 |
| | [Paracoccidioides brasiliensis Pb18] | | |
| EEH38257.1 | 6-phosphogluconate dehydrogenase | 100% | 0.0 |
| | [Paracoccidioides brasiliensis Pb01] | | |
| XP_002479015.1 | 6-phosphogluconate dehydrogenase Gnd1, putative | 98% | 0.0 |
| | [Talaromyces stipitatus ATCC 10500] | | |
| XP_001215029.1 | 6-phosphogluconate dehydrogenase [Aspergillus | 95% | 0.0 |
| | terreus NIH2624] | | |
| O60037.1 | 6-phosphogluconate dehydrogenase, | 98% | 0.0 |
| | decarboxylating [Cunninghamella elegans] | | |
| XP_002174980.1 | 6-phosphogluconate dehydrogenase | 100% | 0.0 |
| | [Schizosaccharomyces japonicus yFS275] | | |
| XP_001558673.1 | 6-phosphogluconate dehydrogenase [Botryotinia | 98% | 0.0 |
| | fuckeliana B05.10] | | |
| XP_964959.1 | 6-phosphogluconate dehydrogenase [Neurospora | 99% | 0.0 |
| | crassa OR74A] | | |
| BAD98151.1 | 6-phosphogluconate dehydrogenase [Ascidia | 98% | 0.0 |
| | sydneiensis samea] | | |
| XP_625090.1 | PREDICTED: similar to 6-phosphogluconate | 97% | 0.0 |
| | dehydrogenase, decarboxylating, partial [Apis | | |
| | mellifera] | | |
| XP_001880085.1 | 6-phosphogluconate dehydrogenase [Laccaria | 97% | 0.0 |
| | bicolor S238N-H82] | | |
| XP_567793.1 | phosphogluconate dehydrogenase (decarboxylating) | 98% | 0.0 |
| | [Cryptococcus neoformans var. neoformans JEC21] | | |
| NP_595095.1 | phosphogluconate dehydrogenase, decarboxylating | 98% | 0.0 |
| | [Schizosaccharomyces pombe] | | |
| YP_828280.1 | 6-phosphogluconate dehydrogenase [Solibacter | 98% | 0.0 |
| | usitatus Ellin6076] | | |
| XP_001932608.1 | 6-phosphogluconate dehydrogenase 1 [Pyrenophora | 98% | 0.0 |
| | tritici-repentis Pt-1C-BFP] | | |
| NP_998717.1, | phosphogluconate dehydrogenase isoform 2, 1 | 97% | 0.0 |
| NP_998618.1 | [Danio rerio] | | |
| XP_972051.1 | PREDICTED: similar to 6-phosphogluconate | 97% | 0.0 |
| | dehydrogenase [Tribolium castaneum] | | |
| XP_001600933.1 | PREDICTED: similar to 6-phosphogluconate | 98% | 0.0 |
| | dehydrogenase [Nasonia vitripennis] | | |
| ZP_01877330.1 | 6-phosphogluconate dehydrogenase [Lentisphaera | 97% | 0.0 |
| | araneosa HTCC2155] | | |
| YP_007316.1 | 6-phosphogluconate dehydrogenase [Candidatus | 97% | 0.0 |
| | Protochlamydia amoebophila UWE25] | | |
| YP_003072132.1 | 6-phosphogluconate dehydrogenase, | 98% | 0.0 |
| | decarboxylating [Teredinibacter turnerae T7901] | | |
| ZP_05103058.1 | 6-phosphogluconate dehydrogenase, | 97% | 0.0 |
| | decarboxylating [Methylophaga thiooxidans] | | |
| NP_501998.1 | 6-phosphogluconate dehydrogenase, | 97% | 0.0 |
| | decarboxylating [Caenorhabditis elegans] | | |
| ZP_05103246.1 | 6-phosphogluconate dehydrogenase, | 97% | 0.0 |
| | decarboxylating [Methylophaga thiooxidans | | |
| | DMS010] | | |
| NP_001006303.1 | phosphogluconate dehydrogenase [Gallus gallus] | 97% | 0.0 |
| ZP_05709847.1 | 6-phosphogluconate dehydrogenase, | 97% | 0.0 |
| | decarboxylating [Desulfurivibrio alkaliphilus AHT2] | | |
| NP_001083291.1 | phosphogluconate dehydrogenase [Xenopus laevis] | 95% | 0.0 |
| ZP_03627847.1 | 6-phosphogluconate dehydrogenase, | 98% | 0.0 |
| | decarboxylating [bacterium Ellin514] | | |
| YP_001983553.1 | 6-phosphogluconate dehydrogenase [Cellvibrio | 98% | 0.0 |
| | japonicus Ueda107] | | |
| NP_002622.2, | phosphogluconate dehydrogenase [Homo sapiens] | 97% | 0.0 |
| AAA75302.1 | | | |
| ZP_03127624.1 | 6-phosphogluconate dehydrogenase, | 98% | 0.0 |
| | decarboxylating [Chthoniobacter flavus Ellin428] | | |
| XP_001651702.1 | 6-phosphogluconate dehydrogenase [Aedes aegypti] | 98% | 0.0 |
| ACN10812.1 | 6-phosphogluconate dehydrogenase, | 97% | 0.0 |
| | decarboxylating [Salmo salar] | | |
| XP_001509796.1 | PREDICTED: similar to Phosphogluconate | 97% | 0.0 |
| | dehydrogenase [Ornithorhynchus anatinus] | | |
| YP_661682.1 | 6-phosphogluconate dehydrogenase | 98% | 0.0 |
| | [Pseudoalteromonas atlantica T6c] | | |
| NP_001009467.1 | phosphogluconate dehydrogenase [Ovis aries] | 97% | 0.0 |

### Example 1

### Over-expression Of Glucose-6-Phosphate Dehydrogenase (“G6PDH”) In Yarrowia lipolytica Strain Y2107U

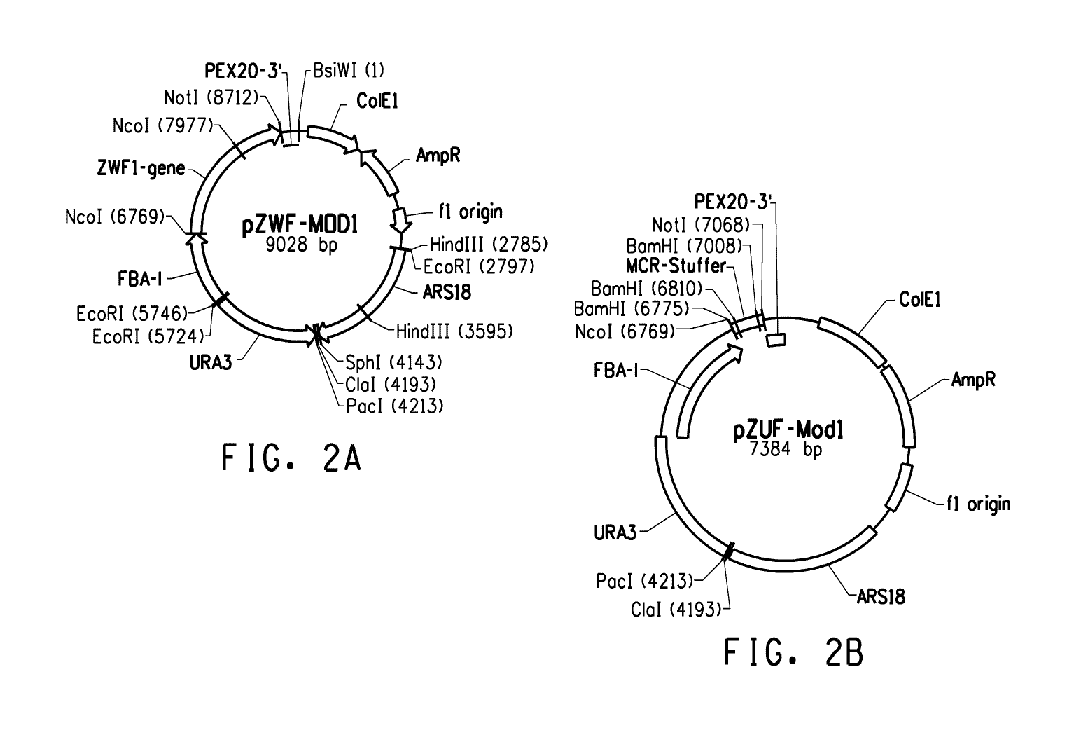

The present Example describes construction of plasmid pZWF-MOD1 (FIG. 2A; SEQ ID NO:7), to enable over-expression of the Yarrowia gene encoding glucose-6-phosphate dehydrogenase [“G6PDH”] under the control of a strong native Yarrowia promoter.

Transformation of the PUFA-producing Y. lipolytica strain Y2107U with the over-expression plasmid was performed, and the effect of the over-expression on cell growth and lipid synthesis was determined and compared. Specifically, over-expression of G6PDH resulted in decreased cell growth.

Construction of Plasmid pZWF-MOD1, Comprising Yarrowia G6PDH

The Yarrowia lipolytica G6PDH ORF contained an intron near the 5′-end (nucleotides 85-524 of SEQ ID NO:10). The nucleotide sequence of the cDNA encoding G6PDH is set forth as SEQ ID NO:1.

Primers YZWF-F1 (SEQ ID NO:8) and YZWF-R (SEQ ID NO:9) were designed for amplification of the coding region of the Yarrowia gene encoding G6PDH. Primer YZWF-F1 contains an inserted 6 bases “GGATCC” (creating a BamHI site) after the translation initiation “ATG” codon. Both genomic DNA and cDNA were used as templates in two separate PCR amplifications (General Methods), such that the coding region of G6PDH was obtained both with and without the 440 bp intron (SEQ ID NO:12).

Amplified DNA fragments were digested with BamHI and NotI, and ligated to BamHI and NotI digested pZUF-MOD1 (SEQ ID NO:13; FIG. 2B). Plasmid pZUF-MOD1 has been previously described in Example 5 of U.S. Pat. No. 7,192,762. The “MCR-Stuffer” fragment in FIG. 2B corresponds to a 253 bp “stuffer” DNA fragment amplified from a portion of pDNR-LIB (ClonTech, Palo Alto, Calif.); this fragment was operably linked to the strong Yarrowia FBAIN promoter (U.S. Pat. No. 7,202,356; SEQ ID NO:14).

Ligation mixtures were used to transform E. coli TOP10 competent cells. No colonies were obtained with the ligation mixture containing amplified cDNA fragments, despite several attempts. Colonies were readily obtained with the amplified genomic DNA fragments. DNA from these colonies was purified with Qiagen Miniprep kits and the identity of the plasmid was confirmed by restriction mapping. The resulting plasmid, comprising a chimeric FBAIN::G6PDH::Pex20 gene, was designated “pZWF-MOD1” (FIG. 2A; SEQ ID NO:7).

Effect Of G6PDH Over-Expression In Yarrowia lipolytica Strain Y2107U

Y. lipolytica strain Y2107U, which collectively refers to strains Y2107U1 and Y2107U2, producing about 16% EPA of total lipids after two-stage growth via expression of a Δ6 desaturase/Δ6 elongase pathway, was generated as described in Example 4 of U.S. Pat. No. 7,192,762, hereby incorporated herein by reference. Briefly, strain Y2107U was derived from Yarrowia lipolytica ATCC #20362, via construction of strain M4 (producing 8% DGLA), strain Y2047 (producing 11% ARA), strain Y2048 (producing 11% EPA), strain Y2060 (producing 13% EPA), strain Y2072 (producing 15% EPA), strain Y2072U1 (producing 14% EPA) and Y2089 (producing 18% EPA). The final genotype of strain Y2107U with respect to wild type Yarrowia lipolytica ATCC #20362 was FBAIN::EL1S:Pex20, GPDIN::EL1S::Lip2, GPAT::EL1S::Pex20, GPAT::EL1S::XPR, TEF::EL2S::XPR, TEF::Δ6S::Lip1, FBAIN::Δ6S::Lip1, FBA::F.Δ12::Lip2, TEF::F.Δ12::Pex16, FBAIN::M.Δ12::Pex20, FBAIN::MAΔ5::Pex20, TEF::MAΔ5::Lip1, TEF::HΔ5S::Pex16, TEF::I.Δ5S::Pex20, GPAT::I.Δ5S::Pex20, TEF::Δ17S::Pex20, FBAIN::Δ17S::Lip2, FBAINm::Δ17S::Pex16, TEF::rELO2S::Pex20 (2 copies). Abbreviations are as follows: EL1S is a codon-optimized elongase 1 gene derived from Mortierella alpina (GenBank Accession No. AX464731); EL2S is a codon-optimized elongase gene derived from Thraustochytrium aureum [U.S. Pat. No. 6,677,145]; Δ65 is a codon-optimized Δ6 desaturase gene derived from Mortierella alpina (GenBank Accession No. AF465281); F.Δ12 is a Fusarium moniliforme Δ12 desaturase gene [U.S. Pat. No. 7,504,259]; M.Δ12 is a Mortierella isabellina Δ12 desaturase gene (GenBank Accession No. AF417245); MAΔ5 is a Mortierella alpina Δ5 desaturase gene (GenBank Accession No. AF067654); HΔ5S is a codon-optimized Δ5 desaturase gene derived from Homo sapiens (GenBank Accession No. NP_{—}037534); I.Δ55 is a codon-optimized Δ5 desaturase gene, derived from Isochrysis galbana (WO 2002/081668); Δ175 is a codon-optimized Δ17 desaturase gene derived from S. diclina [U.S. Pat. No. 7,125,672]; and, rELO2S is a codon-optimized rELO2 C_{16/18} elongase gene derived from rat (GenBank Accession No. AB071986).

Plasmid pZWF-MOD1 (SEQ ID NO:7) and control plasmid pZUF-MOD1 (SEQ ID NO:13) were used to transform strain Y2107U. Transformants were grown in 25 mL SD medium for 2 days at 30° C. and 250 rpm. Cells were then collected by centrifugation and resuspended in HGM medium. The cultures were allowed to grow for 5 more days at 30° C. and 250 rpm.

For dry cell weight determination, 10 mL of each culture were centrifuged at 3750 rpm for 5 min. Each cell pellet was resuspended in 10 mL water and centrifuged again. The cell pellet was then transferred to a pre-weighted aluminum pan, dried at 80° C. overnight and weighted to determine the dry cell weight [“DCW”] from 10 mL cell culture.

For lipid determination, the cells were collected by centrifugation, lipids were extracted, and FAMEs were prepared by trans-esterification, and subsequently analyzed with a Hewlett-Packard 6890 GC (as described in the General Methods).

The DCW, total lipid content of cells [“TFAs % DCW”], and the concentration of EPA as a weight percent of TFAs [“EPA % TFAs”] for three pZUF-MOD1 transformants, comprising the chimeric FBAIN::MCR-Stuffer::Pex20 gene, and nine pZWF-MOD1 transformants, comprising the chimeric FBAIN::G6PDH::Pex20 gene, are shown below in Table 6, with the average of each highlighted in bold text.

More specifically, the term “total fatty acids” [“TFAs”] herein refer to the sum of all cellular fatty acids that can be derivatized to fatty acid methyl esters [“FAMEs”] by the base transesterification method (as known in the art) in a given sample, which may be the biomass or oil, for example. Thus, total fatty acids include fatty acids from neutral lipid fractions (including diacylglycerols, monoacylglycerols and triacylglycerols [“TAGs”]) and from polar lipid fractions (including the phosphatidylcholine and phosphatidylethanolamine fractions) but not free fatty acids.

The term “total lipid content” of cells is a measure of TFAs as a percent of the DCW, although total lipid content can be approximated as a measure of FAMEs as a percent of the DCW [“FAMEs % DCW”]. Thus, total lipid content [“TFAs % DCW”] is equivalent to, e.g., milligrams of total fatty acids per 100 milligrams of DCW.

The concentration of a fatty acid in the total lipid is expressed herein as a weight percent of TFAs [“% TFAs”], e.g., milligrams of the given fatty acid per 100 milligrams of TFAs. Unless otherwise specifically stated in the disclosure herein, reference to the percent of a given fatty acid with respect to total lipids is equivalent to concentration of the fatty acid as % TFAs (e.g., % EPA of total lipids is equivalent to EPA % TFAs).

In some cases, it is useful to express the content of a given fatty acid(s) in a cell as its weight percent of the dry cell weight [“% DCW”]. Thus, for example, eicosapentaenoic acid % DCW would be determined according to the following formula: [(eicosapentaenoic acid % TFAs)*(TFAs % DCW)]/100. The content of a given fatty acid(s) in a cell as its weight percent of the dry cell weight [“% DCW”] can be approximated, however, as: [(eicosapentaenoic acid % TFAs)*(FAMEs % DCW)]/100.

TABLE 6
G6PDH Over-expression In Yarrowia lipolytica Strain Y2107U

| | | DCW | TFAs | EPA % |
|---|---|---|---|---|
| Sample | Plasmid | (g/L) | % DCW | TFAs |
| Control-1 | pZUF-MOD1 | 2.22 | 16 | 13.5 |
| Control-2 | | 1.77 | 15 | 15.9 |
| Control-3 | | 1.85 | 19 | 16.4 |
| Control-Average | pZUF-MOD1 | 1.94 | 16.7 | 15.3 |
| G6PDH-1 | pZWF-MOD1 | 1.8 | 15 | 13.3 |
| G6PDH-2 | | 1.56 | 16 | 16.5 |
| G6PDH-3 | | 1.40 | 19 | 16.4 |
| G6PDH-4 | | 0.35 | nd* | nd* |
| G6PDH-5 | | 1.33 | 16 | 17.8 |
| G6PDH-6 | | 1.02 | 21 | 18.1 |
| G6PDH-7 | | 0.18 | nd* | nd* |
| G6PDH-8 | | 0.17 | nd* | nd* |
| G6PDH-9 | | 0.98 | nd* | nd* |
| G6PDH-Average | pZWF-MOD1 | 0.98 | 17.4 | 16.4 |

*“nd” indicates non-detectable.

The results shown above in Table 6 demonstrated that cells carrying pZWF-MOD1, and expressing the chimeric FBAIN::G6PDH::Pex20 gene, had an average DCW only about half as great as the control. This indicated that the cells over-expressing G6PDH did not grow well. Specifically, some colonies had less than 10% of the DCW. For those colonies having a DCW more than 50% of the control, the total lipid and EPA content was slightly increased when compared to the control values.

On the basis of the results above, and the observed cellular phenotype wherein cells were unable to grow well, it was concluded that over-expression of G6PD alone under the control of a very strong promoter resulted in unacceptable quantities of 6-phosphogluconolactone that inhibit the growth of Yarrowia lipolytica.

### Example 2

Construction of Plasmid pZKLY-PP2, for Coordinately Regulated Over-Expression of Glucose-6-Phosphate Dehydrogenase [“G6PDH”] and 6-Phosphogluconolatonase [“6PGL”]

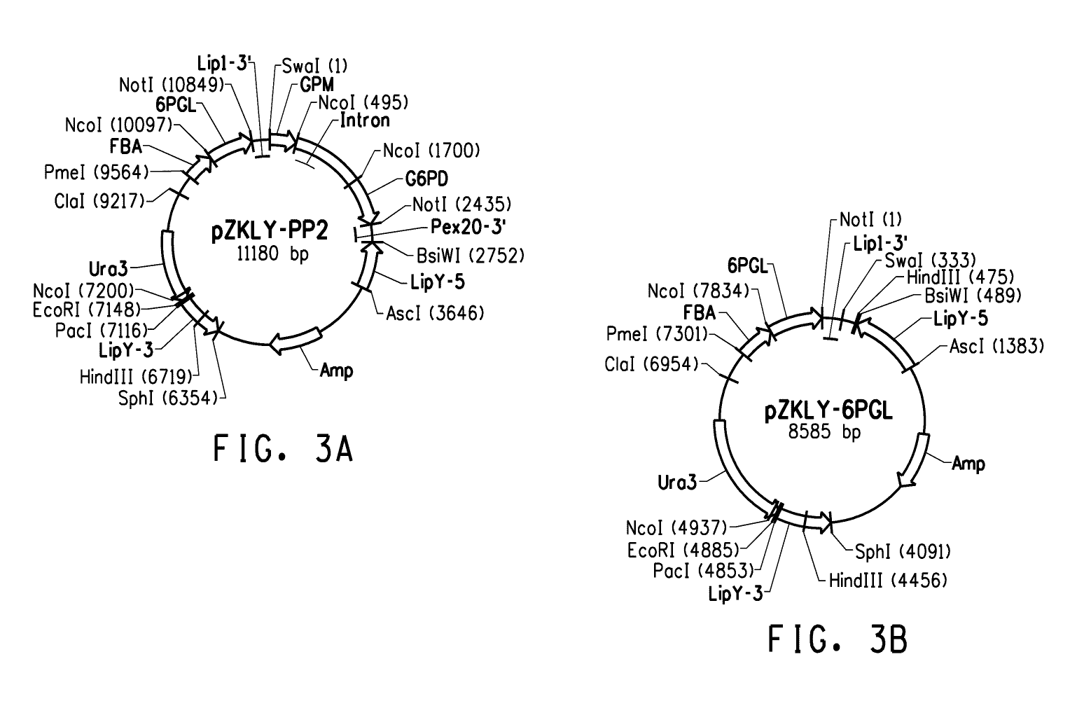

The present Example describes construction of plasmid pZKLY-PP2 (FIG. 3A; SEQ ID NO:15) to over-express the Yarrowia genes encoding glucose-6-phosphate dehydrogenase [“G6PDH”] and 6-phosphogluconolatonase [“6PGL”] in a coordinately regulated fashion. Specifically, a weak native Yarrowia promoter was selected to drive expression of G6PD, while a strong native Yarrowia promoter was operably linked to 6PGL. This strategy was designed to ensure rapid conversion of 6-phosphogluconolactone to 6-phosphogluconate and thereby avoid accumulation of toxic levels of 6-phosphogluconolactone.

Construction of Plasmid pZKLY-PP2 for Over-Expression of G6PDH and 6PGL

Construction of plasmid pZKLY-PP2 first required individual amplification of the Yarrowia 6PGL and G6PDH genes and ligation of each respective gene to a suitable Yarrowia promoter to create an individual expression cassette. The two expression cassettes were then assembled in plasmid pZKLY-PP2 for coordinately regulated over-expression.

Specifically, the Yarrowia 6PGL gene was amplified from Y. lipolytica genomic DNA using PCR primers YL961 (SEQ ID NO:16) and YL962 (SEQ ID NO:17) (General Methods). Primer YL961 contained an inserted three bases “GCT” after the translation initiation “ATG” codon. A 752 bp NcoI/NotI fragment comprising 6PGL and a 533 bp Pmel/NcoI fragment comprising the Yarrowia FBA promoter (U.S. Pat. No. 7,202,356; SEQ ID NO:18) were ligated together with Pmel/NotI digested pZKLY plasmid (SEQ ID NO:25) to produce pZKLY-6PGL (SEQ ID NO:19; FIG. 3B).

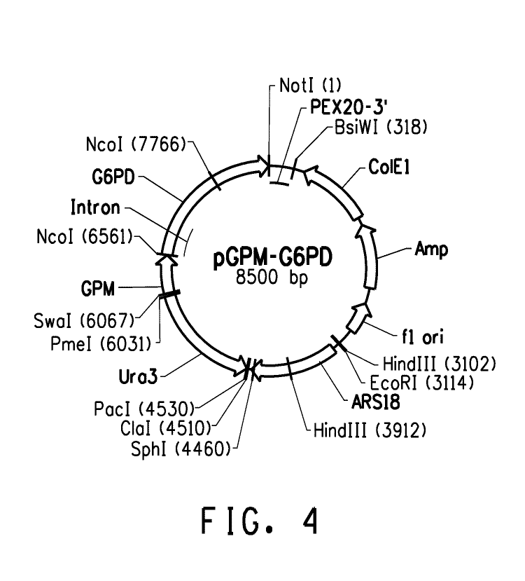

Similarly, the Yarrowia G6PDH was amplified from genomic DNA by PCR using primers YL959 (SEQ ID NO:20) and YL960 (SEQ ID NO:21) (General Methods). Primer YL959 created one base pair mutation within the G6PDH coding region, as the fourth nucleotide “A” was changed to “G” to generate a NcoI site for cloning purposes. Thus, the amplified coding region of G6PDH contained an amino acid change with respect to the wildtype enzyme, such that the second amino acid “Thr” was changed to “Ala”. The PCR product was digested with NcoI/EcoRV to produce a 496 bp fragment, or digested with EcoRV/NotI to produce a 1.4 kB fragment. These two fragments were then ligated together into NcoI/NotI sites of pDMW224-S2 (SEQ ID NO:22) to produce pGPM-G6PD (SEQ ID NO:23; FIG. 4), such that G6PDH was operably linked to the Yarrowia GPM promoter (U.S. Pat. No. 7,259,255; SEQ ID NO:24).

A 2.8 kB fragment comprising GPM::G6PD was subsequently excised from pGPM-G6PD by digestion with SwaI/BsiWI restriction enzymes. The isolated fragment was then cloned into the SwaI/BsiWI sites of pZKLY-6PGL (SEQ ID NO:19; FIG. 3B) to produce pZKLY-PP2.

Thus, plasmid pZKLY-PP2 (FIG. 3A) contained the following components:

TABLE 7
Description of Plasmid pZKL-PP2 (SEQ ID NO: 15)

| RE Sites And | |
|---|---|
| Nucleotides | |
| Within SEQ ID | Description Of |
| NO: 15 | Fragment And Chimeric Gene Components |
| AscI/BsiWI | 887 bp 5′ portion of Yarrowia Lip7 gene (labeled as |
| (3474-2658) | “LipY-5′N” in Figure; GenBank Accession No. |
| | AJ549519) |
| PacI/SphI | 756 bp 3′ portion of Yarrowia Lip7 gene (labeled as |
| (6951-6182) | “LipY-5′N” in Figure; GenBank Accession No. |
| | AJ549519) |
| SwaI/BsiWI | GPM::G6PDH::Pex20, comprising: |
| (1-2752) | GPM: Yarrowia lipolytica GPM promoter (U.S. Pat. No. |
| | 7,259,255); |
| | G6PDH: derived from Yarrowia lipolytica glucose-6- |
| | phosphate dehydrogenase gene (SEQ ID NO: 1; |
| | GenBank Accession No. XM_504275); |
| | Pex20: Pex20 terminator sequence from Yarrowia |
| | Pex20 gene (GenBank Accession No. AF054613) |
| PmeI/SwaI | FBA::6PGL::Lip1 comprising: |
| (9217-1) | FBA: Yarrowia lipolytica FBA promoter (U.S. Pat. No. |
| | 7,202,356); |
| | 6PGL: derived from Yarrowia lipolytica 6- |
| | phosphogluconolatonase gene (SEQ ID NO: 3; GenBank |
| | Accession No. XM_503830) |
| | Lip1: Lip1 terminator sequence from Yarrowia Lip1 |
| | gene (GenBank Accession No. Z50020) |
| SalI/EcoRI | Yarrowia Ura3 gene |
| (8767-7148) | (GenBank Accession No. AJ306421) |

### Example 3

### Coordinately Regulated Over-Expression of Glucose-6-Phosphate Dehydrogenase [“G6PDH”] and 6-Phosphogluconolatonase [“6PGL”] in Yarrowia lipolytica Strain Y4305U Increases Total Lipids Accumulated

The present Example describes transformation of PUFA-producing Y. lipolytica strain Y4305U with plasmid pZKLY-PP2 and the effect of coordinately regulated over-expression of G6PDH and 6PGL on cell growth and lipid synthesis. Specifically, coordinately regulated over-expression of G6PDH and 6PGL resulted in an increased amount of total lipid, as a percent of DCW, and an increased amount of PUFAs, as a percent of TFAs, in the transformant cells.

Y. lipolytica strain Y4305U (General Methods) was transformed with an 8.5 kB AscI/SphI fragment of pZKLY-PP2 (SEQ ID NO:15; Example 2), according to the General Methods. Transformants were selected on SD media plates lacking uracil. Three pZKLY-PP2 transformants were designated as strains PP12, PP13 and PP14.

For lipid analysis, pZKLY-PP2 transformants and Y4305 cells (control) were grown under comparable oleaginous conditions. Cultures of each strain were first grown at a starting OD₆₀₀ of ˜0.1 in 25 mL of SD media in a 125 mL flask for 48 hrs. The cells were harvested by centrifugation for 5 min at 4300 rpm in a 50 mL conical tube. The supernatant was discarded, and the cells were re-suspended in 25 mL of HGM and transferred to a new 125 mL flask. The cells were incubated with aeration for an additional 120 hrs at 30° C. HGM cultured cells (1 mL) were collected by centrifugation for 1 min at 13,000 rpm, total lipids were extracted, and fatty acid methyl esters (FAMEs) were prepared by trans-esterification, and subsequently analyzed with a Hewlett-Packard 6890 GC (General Methods).

Dry cell weight [“DCW”], total lipid content [“TFAs % DCW”], concentration of a given fatty acid(s) expressed as a weight percent of total fatty acids [“% TFAs”], and content of a given fatty acid(s) as its percent of the dry cell weight [“% DCW”] are shown below in Table 8. Specifically, fatty acids are identified as 18:0 (stearic acid), 18:1 (oleic acid), 18:2 (linoleic acid; ω-6), eicosatetraenoic acid [“ETA”; 20:4 ω-3] and eicosapentaenoic acid [“EPA”; 20:5 ω-3]. The average fatty acid composition of triplicate samples of pZKLY-PP2 transformants of Y. lipolytica Y4305U (i.e., PP12, PP13 and PP14) and Y4305 control strains are highlighted in gray and indicated with “Ave”.

TABLE 8
Lipid Content And Composition In Y. lipolytica Strain Y4305U With Coordinately Regulated
Over-expression Of G6PDH And 6PGL In SD/HGM Medium

| | | | % TFAs | | | | | | |
|---|---|---|---|---|---|---|---|---|---|
| | DCW | TFAs % | 18:0 | 18:1 | 18:2 | 20:4 | 20:5 | EPA | ETA + EPA |
| Sample | (g/L) | DCW | Stearic | Oleic | Linoleic | ETA | EPA | % DCW | % DCW |
| Y4305-1 | 2.50 | 35 | 1.3 | 5.1 | 18.6 | 1.8 | 47.7 | 16.6 | 17.2 |
| Y4305-2 | 2.60 | 33 | 1.3 | 5.0 | 18.6 | 1.9 | 47.9 | 16.0 | 16.6 |
| Y4305-3 | 2.46 | 34 | 1.3 | 5.1 | 18.7 | 1.9 | 47.6 | 16.4 | 17.0 |
| Y4305 Avg | 2.52 | 34 | 1.3 | 5.1 | 18.6 | 1.9 | 47.7 | 16.3 | 16.9 |
| PP12-1 | 2.30 | 38 | 1.2 | 5.7 | 18.7 | 1.8 | 45.6 | 17.5 | 18.2 |
| PP12-2 | 2.36 | 37 | 1.3 | 5.8 | 19.0 | 1.8 | 46.1 | 17.1 | 17.8 |
| PP12-3 | 1.68 | 37 | 1.2 | 5.1 | 18.6 | 1.8 | 47.5 | 17.5 | 18.2 |
| PP12 Avg | 2.11 | 38 | 1.2 | 5.5 | 18.8 | 1.8 | 46.4 | 17.4 | 18.1 |
| PP13-1 | 1.86 | 37 | 1.3 | 5.7 | 19.4 | 1.9 | 45.3 | 16.7 | 17.4 |
| PP13-2 | 1.92 | 38 | 1.3 | 5.7 | 19.2 | 1.9 | 45.3 | 17.1 | 17.8 |
| PP13-3 | 1.88 | 40 | 1.3 | 5.8 | 19.0 | 2.0 | 44.2 | 17.8 | 18.6 |
| PP13 Avg | 1.89 | 38 | 1.3 | 5.7 | 19.2 | 2.0 | 44.9 | 17.2 | 18.0 |
| PP14-1 | 1.72 | 38 | 1.3 | 5.6 | 18.8 | 2.0 | 45.3 | 17.1 | 17.9 |
| PP14-2 | 1.72 | 37 | 1.4 | 5.7 | 19.0 | 2.0 | 45.0 | 16.5 | 17.3 |
| PP14-3 | 1.64 | 39 | 1.3 | 5.7 | 18.9 | 2.0 | 45.2 | 17.7 | 18.5 |
| PP14 Avg | 1.69 | 38 | 1.3 | 5.7 | 18.9 | 2.0 | 45.2 | 17.1 | 17.9 |
| PP12, PP13 | 1.89 | 38 | 1.3 | 5.6 | 19.0 | 2.0 | 45.5 | 17.2 | 18.0 |
| and PP14 | | | | | | | | | |
| Avg | | | | | | | | | |

The results in Table 8 showed that over-expression of PP pathway enzymes G6PDH and 6PGL in Y4305U increased the total lipid content [“TFAs % DCW”] by about 12%, compared to the percentage in the control strain Y4305. Also, the EPA productivity [“EPA % DCW”] and ETA+EPA productivity [“ETA+EPA % DCW”] increased about 6-7% in the transformant strains. The EPA titer, measured as “EPA % TFAs”, was slightly diminished in the PP12, PP13 and PP14 strains.

The Y. lipolytica Y4305U pZKLY-PP2 transformants PP12, PP13 and PP14 were also evaluated when grown in an alternate medium. Each strain was grown in 25 mL of FM medium in a 125 mL flask at 30° C. and 250 rpm for 48 hrs. Following centrifugation of 5 mL of each culture at 3600 rpm in a Beckman GS-6R centrifuge, cells were resuspended in 25 mL HGM medium in 125 mL flasks and allowed to grow for 5 days at 30° C. and 250 rpm.

Cells from each culture were harvested by centrifugation and total lipids were extracted, and FAMEs were prepared by trans-esterification, and subsequently analyzed with a Hewlett-Packard 6890 GC. Results are shown in Table 9, using similar quantification as that described in Table 8.

TABLE 9
Lipid Content And Composition In Y. lipolytica Strain Y4305U
With Coordinately Regulated Over-expression Of G6PDH and 6PGL In FM/HGM Medium

| | DCW | TFAs % | 18:0 | 18:1 | 18:2 | 20:4 | 20:5 | EPA | ETA + EPA |
|---|---|---|---|---|---|---|---|---|---|
| Sample | (g/L) | DCW | Stearic | Oleic | Linoleic | ETA | EPA | % DCW | % DCW |
| Y4305-1 | 4.33 | 28.45 | 1.1 | 5.56 | 18.95 | 1.93 | 46.57 | 13.25 | 13.80 |
| Y4305-2 | 4.09 | 28.87 | 1.1 | 5.49 | 18.87 | 1.93 | 46.24 | 14.35 | 13.91 |
| Y4305 Avg | 4.21 | 28.66 | 1.1 | 5.53 | 18.91 | 1.93 | 46.40 | 13.00 | 13.86 |
| PP12 | 4.05 | 32.16 | 1.56 | 6.72 | 19.62 | 1.98 | 48.93 | 15.74 | 16.37 |
| PP13 | 4.28 | 30.89 | 1.42 | 6.38 | 19.33 | 2.06 | 49.16 | 15.18 | 15.82 |
| PP14 | 4.26 | 28.56 | 1.41 | 5.59 | 18.63 | 2.02 | 50.84 | 14.52 | 15.10 |
| PP12, PP13 | 4.20 | 30.53 | 1.46 | 6.23 | 19.20 | 2.02 | 49.64 | 15.15 | 15.76 |
| and PP14 | | | | | | | | | |
| Avg | | | | | | | | | |

The results in Table 9 showed that coordinately regulated over-expression of the PP pathway enzymes G6PDH and 6PGL in Y4305U increased the total lipid content [“TFAs % DCW”], the EPA productivity [“EPA % DCW”] and ETA+EPA productivity [“ETA+EPA % DCW”], as well as the EPA titer [“EPA % TFAs”]. This effect is attributed to the increased availability of cellular NADPH, generated by G6PDH.

What is claimed is:

1. A transgenic microorganism comprising:
(a) at least one gene encoding glucose-6-phosphate dehydrogenase;
(b) at least one gene encoding 6-phosphogluconolactonase; and,
(c) at least one heterologous gene encoding a non-native product of interest;
wherein biosynthesis of the non-native product of interest comprises at least one enzymatic reaction that requires nicotinamide adenine dinucleotide phosphate;
wherein coordinately regulated over-expression of (a) and (b) results in an increased quantity of nicotinamide adenine dinucleotide phosphate; and,
wherein the increased quantity of nicotinamide adenine dinucleotide phosphate results in an increased quantity of the product of interest produced by expression of (c) in the transgenic microorganism, when compared to the quantity of nicotinamide adenine dinucleotide phosphate and the quantity of the product of interest produced by a transgenic microorganism comprising (c) and either lacking or not over-expressing (a) and (b) in a coordinately regulated fashion.

2. The transgenic microorganism of claim 1, wherein coordinately regulated over-expression of the at least one gene encoding glucose-6-phosphate dehydrogenase and the at least one gene encoding 6-phosphogluconolactonase is achieved by a means selected from the group consisting of:
(a) the at least one gene encoding glucose-6-phosphate dehydrogenase is operably linked to a first promoter and the at least one gene encoding 6-phosphogluconolactonase is operably linked to a second promoter, wherein the first promoter has equivalent or reduced activity when compared to the second promoter;
(b) the at least one gene encoding glucose-6-phosphate dehydrogenase is expressed in multicopy and the at least one gene encoding 6-phosphogluconolactonase is expressed in multicopy, wherein the copy number of the at least one gene encoding glucose-6-phosphate dehydrogenase is equivalent or reduced when compared to the copy number of the at least one gene encoding 6-phosphogluconolactonase;
(c) the enzymatic activity of the at least one gene encoding glucose-6-phosphate dehydrogenase is linked to the enzymatic activity of the at least one gene encoding 6-phosphogluconolactonase as a multizyme; and,
(d) a combination of any of the means set forth in (a), (b) and (c).

3. The transgenic microorganism of claim 1, wherein at least one gene encoding 6-phosphogluconate dehydrogenase is expressed in addition to the genes of (a), (b) and (c).

4. The transgenic microorganism of claim 1, wherein the non-native product of interest is selected from the group consisting of: polyunsaturated fatty acids, carotenoids, amino acids, vitamins, sterols, flavonoids, organic acids, polyols and hydroxyesters.

5. The transgenic microorganism of claim 4, wherein: the non-native product of interest is selected from the group consisting of: an omega-3 fatty acid and an omega-6 fatty acid; and, the at least one heterologous gene of (c) is selected from the group consisting of: delta-12 desaturase, delta-6 desaturase, delta-8 desaturase, delta-5 desaturase, delta-17 desaturase, delta-15 desaturase, delta-9 desaturase, delta-4 desaturase, C_{14/16} elongase, C_{16/18} elongase, C_{18/20} elongase, C_{20/22} elongase and delta-9 elongase.

6. The transgenic microorganism of claim 1, wherein the microorganism is selected from the group consisting of: algae, yeast, euglenoids, stramenopiles, oomycetes and fungi.

7. The transgenic microorganism of claim 6, wherein the yeast is an oleaginous yeast.

8. A transgenic oleaginous yeast comprising:
(a) at least one gene encoding glucose-6-phosphate dehydrogenase;
(b) at least one gene encoding 6-phosphogluconolactonase; and,
(c) at least one heterologous gene encoding a non-native product of interest, wherein the product of interest is selected from the group consisting of: at least one polyunsaturated fatty acid, at least one quinone-derived compound, at least one carotenoid and at least one sterol;
wherein coordinately regulated over-expression of (a) and (b) results in an increased quantity of nicotinamide adenine dinucleotide phosphate; and,
wherein the increased quantity of nicotinamide adenine dinucleotide phosphate results in an increased quantity of the product of interest produced by expression of (c) in the transgenic oleaginous yeast when compared to the quantity of nicotinamide adenine dinucleotide phosphate and the quantity of the product of interest produced by a transgenic oleaginous yeast comprising (c) and either lacking or not over-expressing (a) and (b) in a coordinately regulated fashion.

9. The transgenic oleaginous yeast of claim 8 wherein the oleaginous yeast is Yarrowia lipolytica.

10. The transgenic oleaginous yeast of claim 8 or 9 wherein the at least one polyunsaturated fatty acid is selected from the group consisting of: linoleic acid, gamma-linolenic acid, eicosadienoic acid, dihomo-gamma-linolenic acid, arachidonic acid, docosatetraenoic acid, omega-6 docosapentaenoic acid, alpha-linolenic acid, stearidonic acid, eicosatrienoic acid, eicosatetraenoic acid, eicosapentaenoic acid, omega-3 docosapentaenoic acid and docosahexaenoic acid.

11. The transgenic oleaginous yeast of claim 10 wherein total lipid content is increased in addition to the quantity of nicotinamide adenine dinucleotide phosphate and the quantity of the at least one polyunsaturated fatty acid, when compared to the total lipid content produced by a transgenic oleaginous yeast comprising (c) and either lacking or not over-expressing (a) and (b) in a coordinately regulated fashion.

12. The transgenic oleaginous yeast of claim 8 wherein the at least one carotenoid is selected from the group consisting of: antheraxanthin, adonirubin, adonixanthin, astaxanthin, canthaxanthin, capsorubrin, β-cryptoxanthin, α-carotene, β-carotene, β,ψ-carotene, δ-carotene, ε-carotene, echinenone, 3-hydroxyechinenone, 3′-hydroxyechinenone, γ-carotene, ψ-carotene, 4-keto-γ-carotene, ζ-carotene, α-cryptoxanthin, deoxyflexixanthin, diatoxanthin, 7,8-didehydroastaxanthin, didehydrolycopene, fucoxanthin, fucoxanthinol, isorenieratene, β-isorenieratene, lactucaxanthin, lutein, lycopene, myxobactone, neoxanthin, neurosporene, hydroxyneurosporene, peridinin, phytoene, phytofluene, rhodopin, rhodopin glucoside, 4-keto-rubixanthin, siphonaxanthin, spheroidene, spheroidenone, spirilloxanthin, torulene, 4-keto-torulene, 3-hydroxy-4-keto-torulene, uriolide, uriolide acetate, violaxanthin, zeaxanthin-β-diglucoside, zeaxanthin, a C30 carotenoid, and combinations thereof.

13. The transgenic oleaginous yeast of claim 8 wherein the at least one quinone derived compound is selected from the group consisting of: a ubiquinone, a vitamin K compound, and a vitamin E compound, and combinations thereof.

14. The transgenic oleaginous yeast of claim 8 wherein the at least one sterol compound is selected from the group consisting of: squalene, lanosterol, zymosterol, ergosterol, 7-dehydrocholesterol (provitamin D3), and combinations thereof.

15. A method for the production of a non-native product of interest comprising:
(a) providing a transgenic microorganism comprising:
(i) at least one gene encoding glucose-6-phosphate dehydrogenase;
(ii) at least one gene encoding 6-phosphogluconolactonase; and,
(iii) at least one heterologous gene encoding a non-native product of interest;
wherein (i) and (ii) are over-expressed in a coordinately regulated fashion and wherein an increased quantity of nicotinamide adenine dinucleotide phosphate is produced when compared to the quantity of nicotinamide adenine dinucleotide phosphate produced by a transgenic microorganism either lacking or not over-expressing (i) and (ii) in a coordinately regulated fashion;
(b) growing the transgenic microorganism of step (a) in the presence of a fermentable carbon source whereby expression of (iii) results in production of the non-native product of interest; and
(c) optionally recovering the non-native product of interest.


SEQUENCE LISTING (25 sequences)

SEQ ID NO: 1 - DNA, 1497 bp, Yarrowia lipolytica
  CDS (1)..(1497): GenBank Accession No. XM_504275
atgactggcaccttacccaagttcggcgacggaaccaccattgtggttcttggagcctccggcgacctcgctaagaagaagaccttccccgccctcttcggcctttaccgaaacggcctgctgcccaaaaatgttgaaatcatcggctacgcacggtcgaaaatgactcaggaggagtaccacgagcgaatcagccactacttcaagacccccgacgaccagtccaaggagcaggccaagaagttccttgagaacacctgctacgtccagggcccttacgacggtgccgagggctaccagcgactgaatgaaaagattgaggagtttgagaagaagaagcccgagccccactaccgtcttttctacctggctctgccccccagcgtcttccttgaggctgccaacggtctgaagaagtatgtctaccccggcgagggcaaggcccgaatcatcatcgagaagccctttggccacgacctggcctcgtcacgagagctccaggacggccttgctcctctctggaaggagtctgagatcttccgaatcgaccactacctcggaaaggagatggtcaagaacctcaacattctgcgatttggcaaccagttcctgtccgccgtgtgggacaagaacaccatttccaacgtccagatctccttcaaggagccctttggcactgagggccgaggtggatacttcaacgacattggaatcatccgagacgttattcagaaccatctgttgcaggttctgtccattctagccatggagcgacccgtcactttcggcgccgaggacattcgagatgagaaggtcaaggtgctccgatgtgtcgacattctcaacattgacgacgtcattctcggccagtacggcccctctgaagacggaaagaagcccggatacaccgatgacgatggcgttcccgatgactcccgagctgtgacctttgctgctctccatctccagatccacaacgacagatgggagggtgttcctttcatcctccgagccggtaaggctctggacgagggcaaggtcgagatccgagtgcagttccgagacgtgaccaagggcgttgtggaccatctgcctcgaaatgagctcgtcatccgaatccagccctccgagtccatctacatgaagatgaactccaagctgcctggccttactgccaagaacattgtcaccgacctggatctgacctacaaccgacgatactcggacgtgcgaatccctgaggcttacgagtctctcattctggactgcctcaagggtgaccacaccaactttgtgcgaaacgacgagctggacatttcctggaagattttcaccgatctgctgcacaagattgacgaggacaagagcattgtgcccgagaagtacgcctacggctctcgtggccccgagcgactcaagcagtggctccgagaccgaggctacgtgcgaaacggcaccgagctgtaccaatggcctgtcaccaagggctcctcgtga

SEQ ID NO: 2 - protein, 498 aa, Yarrowia lipolytica
MTGTLPKFGDGTTIVVLGASGDLAKKKTFPALFGLYRNGLLPKNVEIIGYARSKMTQEEYHERISHYFKTPDDQSKEQAKKFLENTCYVQGPYDGAEGYQRLNEKIEEFEKKKPEPHYRLFYLALPPSVFLEAANGLKKYVYPGEGKARIIIEKPFGHDLASSRELQDGLAPLWKESEIFRIDHYLGKEMVKNLNILRFGNQFLSAVWDKNTISNVQISFKEPFGTEGRGGYFNDIGIIRDVIQNHLLQVLSILAMERPVTFGAEDIRDEKVKVLRCVDILNIDDVILGQYGPSEDGKKPGYTDDDGVPDDSRAVTFAALHLQIHNDRWEGVPFILRAGKALDEGKVEIRVQFRDVTKGVVDHLPRNELVIRIQPSESIYMKMNSKLPGLTAKNIVTDLDLTYNRRYSDVRIPEAYESLILDCLKGDHTNFVRNDELDISWKIFTDLLHKIDEDKSIVPEKYAYGSRGPERLKQWLRDRGYVRNGTELYQWPVTKGSS

SEQ ID NO: 3 - DNA, 747 bp, Yarrowia lipolytica
  CDS (1)..(747): GenBank Accession No. XM_503830
atgcccaaggtcatctctaagaacgaatcgcaactggtcgctgaggctgctgccgctgagatcattcgactccagaacgagtcaattgctgccactggagctttccatgttgccgtatctggaggctctctggtgtctgctctccgaaagggtctggtcaacaactcggagaccaagttccccaagtggaagattttcttctccgacgaacggctggtcaagctggacgatgccgactccaactacggtctcctcaagaaggatctgctcgatcacatccccaaggatcagcaaccacaggtcttcaccgtcaaggagtctcttctgaacgactctgatgccgtctccaaggactaccaggagcagattgtcaagaatgtgcctctcaacggccagggagtgcctgttttcgatctcattctgctcggatgcggtcctgatggccacacttgctcgctgttccctggacacgctctgctcaaggaggagaccaagtttgtcgccaccattgaggactctcccaagcctcctcctcgacgaatcaccatcactttccccgttctcaaggctgccaaggccatcgctttcgtcgccgagggagccggaaaggcccctgtcctcaagcagatcttcgaggagcccgagcccactcttccctctgccattgtcaacaaggtcgctaccggacccgttttctggtttgtttccgactctgccgttgagggcgtcaacctctccaagatctag

SEQ ID NO: 4 - protein, 248 aa, Yarrowia lipolytica
MPKVISKNESQLVAEAAAAEIIRLQNESIAATGAFHVAVSGGSLVSALRKGLVNNSETKFPKWKIFFSDERLVKLDDADSNYGLLKKDLLDHIPKDQQPQVFTVKESLLNDSDAVSKDYQEQIVKNVPLNGQGVPVFDLILLGCGPDGHTCSLFPGHALLKEETKFVATIEDSPKPPPRRITITFPVLKAAKAIAFVAEGAGKAPVLKQIFEEPEPTLPSAIVNKVATGPVFWFVSDSAVEGVNLSKI

SEQ ID NO: 5 - DNA, 1470 bp, Yarrowia lipolytica
  CDS (1)..(1470): GenBank Accession No. XM_500938
atgactgacacttcaaacatcaagcctgtcgctgacattgccctcatcggtctcgccgtcatgggccagaacctgatcctcaacatggccgaccacggctacgaggttgttgcctacaaccgaaccacctccaaggtcgaccacttcctcgagaacgaggccaagggaaagtccattattggtgctcactctatcaaggagctgtgtgctctgctgaagcgaccccgacgaatcattctgctcgttaaggccggtgctgctgtcgattctttcatcgaacagctcctgccctatctcgataagggtgatatcatcattgacggtggtaactcccacttccccgactccaaccgacgatacgaggagcttaacgagaagggaatcctctttgttggttccggtgtttccggcggtgaggagggtgcccgatacggtccctccatcatgcccggtggaaacaaggaggcctggccccacattaagaagattttccaggacatctctgctaaggctgatggtgagccctgctgtgactgggtcggtgacgctggtgccggccactttgtcaagatggttcacaacggtattgagtatggtgacatgcagcttatctgcgaggcttacgacctcatgaagcgaggtgctggtttcaccaatgaggagattggagacgttttcgccaagtggaacaacggtatcctcgactccttcctcattgagatcacccgagacatcttcaagtacgacgacggctctggaactcctctcgttgagaagatctccgacactgctggccagaagggtactggaaagtggaccgctatcaacgctcttgaccttggtatgcccgtcaccctgatcggtgaggccgtcttcgctcgatgcctttctgccctcaagcaggagcgtgtccgagcttccaaggttcttgatggccccgagcccgtcaagttcactggtgacaagaaggagtttgtcgaccagctcgagcaggccctttacgcctccaagatcatctcttacgcccagggtttcatgcttatccgagaggccgccaagacctacggctgggagctcaacaacgccggtattgccctcatgtggcgaggtggttgcatcatccgatccgtcttccttgctgacatcaccaaggcttaccgacaggaccccaacctcgagaacctgctgttcaacgacttcttcaagaacgccatctccaaggccaacccctcttggcgagctaccgtggccaaggctgtcacctggggtgttcccactcccgcctttgcctcggctctggctttctacgacggttaccgatctgccaagctccccgctaacctgctccaggcccagcgagactacttcggcgcccacacctaccagctcctcgatggtgatggaaagtggatccacaccaactggaccggccgaggtggtgaggtttcttcttccacttacgatgcttaa

SEQ ID NO: 6 - protein, 489 aa, Yarrowia lipolytica
MTDTSNIKPVADIALIGLAVMGQNLILNMADHGYEVVAYNRTTSKVDHFLENEAKGKSIIGAHSIKELCALLKRPRRIILLVKAGAAVDSFIEQLLPYLDKGDIIIDGGNSHFPDSNRRYEELNEKGILFVGSGVSGGEEGARYGPSIMPGGNKEAWPHIKKIFQDISAKADGEPCCDWVGDAGAGHFVKMVHNGIEYGDMQLICEAYDLMKRGAGFTNEEIGDVFAKWNNGILDSFLIEITRDIFKYDDGSGTPLVEKISDTAGQKGTGKWTAINALDLGMPVTLIGEAVFARCLSALKQERVRASKVLDGPEPVKFTGDKKEFVDQLEQALYASKIISYAQGFMLIREAAKTYGWELNNAGIALMWRGGCIIRSVFLADITKAYRQDPNLENLLFNDFFKNAISKANPSWRATVAKAVTWGVPTPAFASALAFYDGYRSAKLPANLLQAQRDYFGAHTYQLLDGDGKWIHTNWTGRGGEVSSSTYDA

SEQ ID NO: 7 - DNA, 9028 bp, Artificial Sequence
  Plasmid pZWF-MOD1
gtacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccgggccccccctcgaggtcgatggtgtcgataagcttgatatcgaattcatgtcacacaaaccgatcttcgcctcaaggaaacctaattctacatccgagagactgccgagatccagtctacactgattaattttcgggccaataatttaaaaaaatcgtgttatataatattatatgtattatatatatacatcatgatgatactgacagtcatgtcccattgctaaatagacagactccatctgccgcctccaactgatgttctcaatatttaaggggtcatctcgcattgtttaataataaacagactccatctaccgcctccaaatgatgttctcaaaatatattgtatgaacttatttttattacttagtattattagacaacttacttgctttatgaaaaacacttcctatttaggaaacaatttataatggcagttcgttcatttaacaatttatgtagaataaatgttataaatgcgtatgggaaatcttaaatatggatagcataaatgatatctgcattgcctaattcgaaatcaacagcaacgaaaaaaatcccttgtacaacataaatagtcatcgagaaatatcaactatcaaagaacagctattcacacgttactattgagattattattggacgagaatcacacactcaactgtctttctctcttctagaaatacaggtacaagtatgtactattctcattgttcatacttctagtcatttcatcccacatattccttggatttctctccaatgaatgacattctatcttgcaaattcaacaattataataagatataccaaagtagcggtatagtggcaatcaaaaagcttctctggtgtgcttctcgtatttatttttattctaatgatccattaaaggtatatatttatttcttgttatataatccttttgtttattacatgggctggatacataaaggtattttgatttaattttttgcttaaattcaatcccccctcgttcagtgtcaactgtaatggtaggaaattaccatacttttgaagaagcaaaaaaaatgaaagaaaaaaaaaatcgtatttccaggttagacgttccgcagaatctagaatgcggtatgcggtacattgttcttcgaacgtaaaagttgcgctccctgagatattgtacatttttgcttttacaagtacaagtacatcgtacaactatgtactactgttgatgcatccacaacagtttgttttgtttttttttgttttttttttttctaatgattcattaccgctatgtatacctacttgtacttgtagtaagccgggttattggcgttcaattaatcatagacttatgaatctgcacggtgtgcgctgcgagttacttttagcttatgcatgctacttgggtgtaatattgggatctgttcggaaatcaacggatgctcaatcgatttcgacagtaattaattaagtcatacacaagtcagctttcttcgagcctcatataagtataagtagttcaacgtattagcactgtacccagcatctccgtatcgagaaacacaacaacatgccccattggacagatcatgcggatacacaggttgtgcagtatcatacatactcgatcagacaggtcgtctgaccatcatacaagctgaacaagcgctccatacttgcacgctctctatatacacagttaaattacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggactaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtgacaccggtactggtgcttgacagtgttgccaatatctgcgaactttctgtcctcgaacaggaagaaaccgtgcttaagagcaagttccttgagggggagcacagtgccggcgtaggtgaagtcgtcaatgatgtcgatatgggttttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcgtcgcctttgccgacaaaaatgtgatcatgatgaaagccagcaatgacgttgcagctgatattgttgtcggccaaccgcgccgaaaacgcagctgtcagacccacagcctccaacgaagaatgtatcgtcaaagtgatccaagcacactcatagttggagtcgtactccaaaggcggcaatgacgagtcagacagatactcgtcgactcaggcgacgacggaattcctgcagcccatctgcagaattcaggagagaccgggttggcggcgtatttgtgtcccaaaaaacagccccaattgccccggagaagacggccaggccgcctagatgacaaattcaacaactcacagctgactttctgccattgccactaggggggggcctttttatatggccaagccaagctctccacgtcggttgggctgcacccaacaataaatgggtagggttgcaccaacaaagggatgggatggggggtagaagatacgaggataacggggctcaatggcacaaataagaacgaatactgccattaagactcgtgatccagcgactgacaccattgcatcatctaagggcctcaaaactacctcggaactgctgcgctgatctggacaccacagaggttccgagcactttaggttgcaccaaatgtcccaccaggtgcaggcagaaaacgctggaacagcgtgtacagtttgtcttaacaaaaagtgagggcgctgaggtcgagcagggtggtgtgacttgttatagcctttagagctgcgaaagcgcgtatggatttggctcatcaggccagattgagggtctgtggacacatgtcatgttagtgtacttcaatcgccccctggatatagccccgacaataggccgtggcctcatttttttgccttccgcacatttccattgctcggtacccacaccttgcttctcctgcacttgccaaccttaatactggtttacattgaccaacatcttacaagcggggggcttgtctagggtatatataaacagtggctctcccaatcggttgccagtctcttttttcctttctttccccacagattcgaaatctaaactacacatcacacaatgcctgttactgacgtccttaagcgaaagtccggtgtcatcgtcggcgacgatgtccgagccgtgagtatccacgacaagatcagtgtcgagacgacgcgttttgtgtaatgacacaatccgaaagtcgctagcaacacacactctctacacaaactaacccagctctccatggatccaggcaccttacccaagttcggcgacggaaccaccattgtggttcttggagcctccggcgacctcgctaagaagaagaccgtgagtattgaaccagactgaggtcaattgaagagtaggagagtctgagaacattcgacggacctgattgtgctctggaccactcaattgactcgttgagagccccaatgggtcttggctagccgagtcgttgacttgttgacttgttgagcccagaacccccaacttttgccaccatacaccgccatcaccatgacacccagatgtgcgtgcgtatgtgagagtcaattgttccgtggcaaggcacagcttattccaccgtgttccttgcacaggtggtctttacgctctcccactctatccgagcaataaaagcggaaaaacagcagcaagtcccaacagacttctgctccgaataaggcgtctagcaagtgtgcccaaaactcaattcaaaaatgtcagaaacctgatatcaacccgtcttcaaaagctaaccccagttccccgccctcttcggcctttaccgaaacggcctgctgcccaaaaatgttgaaatcatcggctacgcacggtcgaaaatgactcaggaggagtaccacgagcgaatcagccactacttcaagacccccgacgaccagtccaaggagcaggccaagaagttccttgagaacacctgctacgtccagggcccttacgacggtgccgagggctaccagcgactgaatgaaaagattgaggagtttgagaagaagaagcccgagccccactaccgtcttttctacctggctctgccccccagcgtcttccttgaggctgccaacggtctgaagaagtatgtctaccccggcgagggcaaggcccgaatcatcatcgagaagccctttggccacgacctggcctcgtcacgagagctccaggacggccttgctcctctctggaaggagtctgagatcttccgaatcgaccactacctcggaaaggagatggtcaagaacctcaacattctgcgatttggcaaccagttcctgtccgccgtgtgggacaagaacaccatttccaacgtccagatctccttcaaggagccctttggcactgagggccgaggtggatacttcaacgacattggaatcatccgagacgttattcagaaccatctgttgcaggttctgtccattctagccatggagcgacccgtcactttcggcgccgaggacattcgagatgagaaggtcaaggtgctccgatgtgtcgacattctcaacattgacgacgtcattctcggccagtacggcccctctgaagacggaaagaagcccggatacaccgatgacgatggcgttcccgatgactcccgagctgtgacctttgctgctctccatctccagatccacaacgacagatgggagggtgttcctttcatcctccgagccggtaaggctctggacgagggcaaggtcgagatccgagtgcagttccgagacgtgaccaagggcgttgtggaccatctgcctcgaaatgagctcgtcatccgaatccagccctccgagtccatctacatgaagatgaactccaagctgcctggccttactgccaagaacattgtcaccgacctggatctgacctacaaccgacgatactcggacgtgcgaatccctgaggcttacgagtctctcattctggactgcctcaagggtgaccacaccaactttgtgcgaaacgacgagctggacatttcctggaagattttcaccgatctgctgcacaagattgacgaggacaagagcattgtgcccgagaagtacgcctacggctctcgtggccccgagcgactcaagcagtggctccgagaccgaggctacgtgcgaaacggcaccgagctgtaccaatggcctgtcaccaagggctcctcgtgagcggccgcaagtgtggatggggaagtgagtgcccggttctgtgtgcacaattggcaatccaagatggatggattcaacacagggatatagcgagctacgtggtggtgcgaggatatagcaacggatatttatgtttgacacttgagaatgtacgatacaagcactgtccaagtacaatactaaacatactgtacatactcatactcgtacccgggcaacggtttcacttgagtgcagtggctagtgctcttactcgtacagtgtgcaatactgcgtatcatagtctttgatgtatatcgtattcattcatgttagttgc

SEQ ID NO: 8 - DNA, 27 bp, Artificial Sequence
  Primer YZWF-F1
gatcggatccaggcaccttacccaagt

SEQ ID NO: 9 - DNA, 34 bp, Artificial Sequence
  Primer YZWF-R
gatcgcggccgctcacgaggagcccttggtgaca

SEQ ID NO: 10 - DNA, 1937 bp, Yarrowia lipolytica
  CDS (1)..(84)
  Intron (85)..(524)
  CDS (525)..(1934)
atgactggcaccttacccaagttcggcgacggaaccaccattgtggttcttggagcctccggcgacctcgctaagaagaagaccgtgagtattgaaccagactgaggtcaattgaagagtaggagagtctgagaacattcgacggacctgattgtgctctggaccactcaattgactcgttgagagccccaatgggtcttggctagccgagtcgttgacttgttgacttgttgagcccagaacccccaacttttgccaccatacaccgccatcaccatgacacccagatgtgcgtgcgtatgtgagagtcaattgttccgtggcaaggcacagcttattccaccgtgttccttgcacaggtggtctttacgctctcccactctatccgagcaataaaagcggaaaaacagcagcaagtcccaacagacttctgctccgaataaggcgtctagcaagtgtgcccaaaactcaattcaaaaatgtcagaaacctgatatcaacccgtcttcaaaagctaaccccagttccccgccctcttcggcctttaccgaaacggcctgctgcccaaaaatgttgaaatcatcggctacgcacggtcgaaaatgactcaggaggagtaccacgagcgaatcagccactacttcaagacccccgacgaccagtccaaggagcaggccaagaagttccttgagaacacctgctacgtccagggcccttacgacggtgccgagggctaccagcgactgaatgaaaagattgaggagtttgagaagaagaagcccgagccccactaccgtcttttctacctggctctgccccccagcgtcttccttgaggctgccaacggtctgaagaagtatgtctaccccggcgagggcaaggcccgaatcatcatcgagaagccctttggccacgacctggcctcgtcacgagagctccaggacggccttgctcctctctggaaggagtctgagatcttccgaatcgaccactacctcggaaaggagatggtcaagaacctcaacattctgcgatttggcaaccagttcctgtccgccgtgtgggacaagaacaccatttccaacgtccagatctccttcaaggagccctttggcactgagggccgaggtggatacttcaacgacattggaatcatccgagacgttattcagaaccatctgttgcaggttctgtccattctagccatggagcgacccgtcactttcggcgccgaggacattcgagatgagaaggtcaaggtgctccgatgtgtcgacattctcaacattgacgacgtcattctcggccagtacggcccctctgaagacggaaagaagcccggatacaccgatgacgatggcgttcccgatgactcccgagctgtgacctttgctgctctccatctccagatccacaacgacagatgggagggtgttcctttcatcctccgagccggtaaggctctggacgagggcaaggtcgagatccgagtgcagttccgagacgtgaccaagggcgttgtggaccatctgcctcgaaatgagctcgtcatccgaatccagccctccgagtccatctacatgaagatgaactccaagctgcctggccttactgccaagaacattgtcaccgacctggatctgacctacaaccgacgatactcggacgtgcgaatccctgaggcttacgagtctctcattctggactgcctcaagggtgaccacaccaactttgtgcgaaacgacgagctggacatttcctggaagattttcaccgatctgctgcacaagattgacgaggacaagagcattgtgcccgagaagtacgcctacggctctcgtggccccgagcgactcaagcagtggctccgagaccgaggctacgtgcgaaacggcaccgagctgtaccaatggcctgtcaccaagggctcctcgtga

SEQ ID NO: 11 - protein, 498 aa, Yarrowia lipolytica; identical to SEQ ID NO: 2

SEQ ID NO: 12 - DNA, 440 bp, Yarrowia lipolytica
gtgagtattgaaccagactgaggtcaattgaagagtaggagagtctgagaacattcgacggacctgattgtgctctggaccactcaattgactcgttgagagccccaatgggtcttggctagccgagtcgttgacttgttgacttgttgagcccagaacccccaacttttgccaccatacaccgccatcaccatgacacccagatgtgcgtgcgtatgtgagagtcaattgttccgtggcaaggcacagcttattccaccgtgttccttgcacaggtggtctttacgctctcccactctatccgagcaataaaagcggaaaaacagcagcaagtcccaacagacttctgctccgaataaggcgtctagcaagtgtgcccaaaactcaattcaaaaatgtcagaaacctgatatcaacccgtcttcaaaagctaaccccag

SEQ ID NO: 13 - DNA, 7323 bp, Artificial Sequence
  Plasmid pZUF-MOD-1
gtacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccgggccccccctcgaggtcgatggtgtcgataagcttgatatcgaattcatgtcacacaaaccgatcttcgcctcaaggaaacctaattctacatccgagagactgccgagatccagtctacactgattaattttcgggccaataatttaaaaaaatcgtgttatataatattatatgtattatatatatacatcatgatgatactgacagtcatgtcccattgctaaatagacagactccatctgccgcctccaactgatgttctcaatatttaaggggtcatctcgcattgtttaataataaacagactccatctaccgcctccaaatgatgttctcaaaatatattgtatgaacttatttttattacttagtattattagacaacttacttgctttatgaaaaacacttcctatttaggaaacaatttataatggcagttcgttcatttaacaatttatgtagaataaatgttataaatgcgtatgggaaatcttaaatatggatagcataaatgatatctgcattgcctaattcgaaatcaacagcaacgaaaaaaatcccttgtacaacataaatagtcatcgagaaatatcaactatcaaagaacagctattcacacgttactattgagattattattggacgagaatcacacactcaactgtctttctctcttctagaaatacaggtacaagtatgtactattctcattgttcatacttctagtcatttcatcccacatattccttggatttctctccaatgaatgacattctatcttgcaaattcaacaattataataagatataccaaagtagcggtatagtggcaatcaaaaagcttctctggtgtgcttctcgtatttatttttattctaatgatccattaaaggtatatatttatttcttgttatataatccttttgtttattacatgggctggatacataaaggtattttgatttaattttttgcttaaattcaatcccccctcgttcagtgtcaactgtaatggtaggaaattaccatacttttgaagaagcaaaaaaaatgaaagaaaaaaaaaatcgtatttccaggttagacgttccgcagaatctagaatgcggtatgcggtacattgttcttcgaacgtaaaagttgcgctccctgagatattgtacatttttgcttttacaagtacaagtacatcgtacaactatgtactactgttgatgcatccacaacagtttgttttgtttttttttgttttttttttttctaatgattcattaccgctatgtatacctacttgtacttgtagtaagccgggttattggcgttcaattaatcatagacttatgaatctgcacggtgtgcgctgcgagttacttttagcttatgcatgctacttgggtgtaatattgggatctgttcggaaatcaacggatgctcaatcgatttcgacagtaattaattaagtcatacacaagtcagctttcttcgagcctcatataagtataagtagttcaacgtattagcactgtacccagcatctccgtatcgagaaacacaacaacatgccccattggacagatcatgcggatacacaggttgtgcagtatcatacatactcgatcagacaggtcgtctgaccatcatacaagctgaacaagcgctccatacttgcacgctctctatatacacagttaaattacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggactaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtgacaccggtactggtgcttgacagtgttgccaatatctgcgaactttctgtcctcgaacaggaagaaaccgtgcttaagagcaagttccttgagggggagcacagtgccggcgtaggtgaagtcgtcaatgatgtcgatatgggttttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcgtcgcctttgccgacaaaaatgtgatcatgatgaaagccagcaatgacgttgcagctgatattgttgtcggccaaccgcgccgaaaacgcagctgtcagacccacagcctccaacgaagaatgtatcgtcaaagtgatccaagcacactcatagttggagtcgtactccaaaggcggcaatgacgagtcagacagatactcgtcgactcaggcgacgacggaattcctgcagcccatctgcagaattcaggagagaccgggttggcggcgtatttgtgtcccaaaaaacagccccaattgccccggagaagacggccaggccgcctagatgacaaattcaacaactcacagctgactttctgccattgccactaggggggggcctttttatatggccaagccaagctctccacgtcggttgggctgcacccaacaataaatgggtagggttgcaccaacaaagggatgggatggggggtagaagatacgaggataacggggctcaatggcacaaataagaacgaatactgccattaagactcgtgatccagcgactgacaccattgcatcatctaagggcctcaaaactacctcggaactgctgcgctgatctggacaccacagaggttccgagcactttaggttgcaccaaatgtcccaccaggtgcaggcagaaaacgctggaacagcgtgtacagtttgtcttaacaaaaagtgagggcgctgaggtcgagcagggtggtgtgacttgttatagcctttagagctgcgaaagcgcgtatggatttggctcatcaggccagattgagggtctgtggacacatgtcatgttagtgtacttcaatcgccccctggatatagccccgacaataggccgtggcctcatttttttgccttccgcacatttccattgctcggtacccacaccttgcttctcctgcacttgccaaccttaatactggtttacattgaccaacatcttacaagcggggggcttgtctagggtatatataaacagtggctctcccaatcggttgccagtctcttttttcctttctttccccacagattcgaaatctaaactacacatcacacaatgcctgttactgacgtccttaagcgaaagtccggtgtcatcgtcggcgacgatgtccgagccgtgagtatccacgacaagatcagtgtcgagacgacgcgttttgtgtaatgacacaatccgaaagtcgctagcaacacacactctctacacaaactaacccagctctccatggatccaggcctgttaacggccattacggcctgcaggatccgaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgcggccgcaagtgtggatggggaagtgagtgcccggttctgtgtgcacaattggcaatccaagatggatggattcaacacagggatatagcgagctacgtggtggtgcgaggatatagcaacggatatttatgtttgacacttgagaatgtacgatacaagcactgtccaagtacaatactaaacatactgtacatactcatactcgtacccgggcaacggtttcacttgagtgcagtggctagtgctcttactcgtacagtgtgcaatactgcgtatcatagtctttgatgtatatcgtattcattcatgttagttgc

SEQ ID NO: 14 - DNA, 973 bp, Yarrowia lipolytica
  misc_feature: Promoter FBAIN
aaattgccccggagaagacggccaggccgcctagatgacaaattcaacaactcacagctgactttctgccattgccactaggggggggcctttttatatggccaagccaagctctccacgtcggttgggctgcacccaacaataaatgggtagggttgcaccaacaaagggatgggatggggggtagaagatacgaggataacggggctcaatggcacaaataagaacgaatactgccattaagactcgtgatccagcgactgacaccattgcatcatctaagggcctcaaaactacctcggaactgctgcgctgatctggacaccacagaggttccgagcactttaggttgcaccaaatgtcccaccaggtgcaggcagaaaacgctggaacagcgtgtacagtttgtcttaacaaaaagtgagggcgctgaggtcgagcagggtggtgtgacttgttatagcctttagagctgcgaaagcgcgtatggatttggctcatcaggccagattgagggtctgtggacacatgtcatgttagtgtacttcaatcgccccctggatatagccccgacaataggccgtggcctcatttttttgccttccgcacatttccattgctcggtacccacaccttgcttctcctgcacttgccaaccttaatactggtttacattgaccaacatcttacaagcggggggcttgtctagggtatatataaacagtggctctcccaatcggttgccagtctcttttttcctttctttccccacagattcgaaatctaaactacacatcacacaatgcctgttactgacgtccttaagcgaaagtccggtgtcatcgtcggcgacgatgtccgagccgtgagtatccacgacaagatcagtgtcgagacgacgcgttttgtgtaatgacacaatccgaaagtcgctagcaacacacactctctacacaaactaacccagctctcc

SEQ ID NO: 15 - DNA, 11180 bp, Artificial Sequence
  Plasmid pZKLY-PP2
aaatgcgtttggatagcactagtctatgaggagcgttttatgttgcggtgagggcgattggtgctcatatgggttcaattgaggtggcggaacgagcttagtcttcaattgaggtgcgagcgacacaattgggtgtcacgtggcctaattgacctcgggtcgtggagtccccagttatacagcaaccacgaggtgcatgggtaggagacgtcaccagacaatagggttttttttggactggagagggttgggcaaaagcgctcaacgggctgtttggggagctgtgggggaggaattggcgatatttgtgaggttaacggctccgatttgcgtgttttgtcgctcctgcatctccccatacccatatcttccctccccacctctttccacgataattttacggatcagcaataaggttccttctcctagtttccacgtccatatatatctatgctgcgtcgtccttttcgtgacatcaccaaaacacatacaaccatggctggcaccttacccaagttcggcgacggaaccaccattgtggttcttggagcctccggcgacctcgctaagaagaagaccgtgagtattgaaccagactgaggtcaattgaagagtaggagagtctgagaacattcgacggacctgattgtgctctggaccactcaattgactcgttgagagccccaatgggtcttggctagccgagtcgttgacttgttgacttgttgagcccagaacccccaacttttgccaccatacaccgccatcaccatgacacccagatgtgcgtgcgtatgtgagagtcaattgttccgtggcaaggcacagcttattccaccgtgttccttgcacaggtggtctttacgctctcccactctatccgagcaataaaagcggaaaaacagcagcaagtcccaacagacttctgctccgaataaggcgtctagcaagtgtgcccaaaactcaattcaaaaatgtcagaaacctgatatcaacccgtcttcaaaagctaaccccagttccccgccctcttcggcctttaccgaaacggcctgctgcccaaaaatgttgaaatcatcggctacgcacggtcgaaaatgactcaggaggagtaccacgagcgaatcagccactacttcaagacccccgacgaccagtccaaggagcaggccaagaagttccttgagaacacctgctacgtccagggcccttacgacggtgccgagggctaccagcgactgaatgaaaagattgaggagtttgagaagaagaagcccgagccccactaccgtcttttctacctggctctgccccccagcgtcttccttgaggctgccaacggtctgaagaagtatgtctaccccggcgagggcaaggcccgaatcatcatcgagaagccctttggccacgacctggcctcgtcacgagagctccaggacggccttgctcctctctggaaggagtctgagatcttccgaatcgaccactacctcggaaaggagatggtcaagaacctcaacattctgcgatttggcaaccagttcctgtccgccgtgtgggacaagaacaccatttccaacgtccagatctccttcaaggagccctttggcactgagggccgaggtggatacttcaacgacattggaatcatccgagacgttattcagaaccatctgttgcaggttctgtccattctagccatggagcgacccgtcactttcggcgccgaggacattcgagatgagaaggtcaaggtgctccgatgtgtcgacattctcaacattgacgacgtcattctcggccagtacggcccctctgaagacggaaagaagcccggatacaccgatgacgatggcgttcccgatgactcccgagctgtgacctttgctgctctccatctccagatccacaacgacagatgggagggtgttcctttcatcctccgagccggtaaggctctggacgagggcaaggtcgagatccgagtgcagttccgagacgtgaccaagggcgttgtggaccatctgcctcgaaatgagctcgtcatccgaatccagccctccgagtccatctacatgaagatgaactccaagctgcctggccttactgccaagaacattgtcaccgacctggatctgacctacaaccgacgatactcggacgtgcgaatccctgaggcttacgagtctctcattctggactgcctcaagggtgaccacaccaactttgtgcgaaacgacgagctggacatttcctggaagattttcaccgatctgctgcacaagattgacgaggacaagagcattgtgcccgagaagtacgcctacggctctcgtggccccgagcgactcaagcagtggctccgagaccgaggctacgtgcgaaacggcaccgagctgtaccaatggcctgtcaccaagggctcctcgtgagcggccgcaagtgtggatggggaagtgagtgcccggttctgtgtgcacaattggcaatccaagatggatggattcaacacagggatatagcgagctacgtggtggtgcgaggatatagcaacggatatttatgtttgacacttgagaatgtacgatacaagcactgtccaagtacaatactaaacatactgtacatactcatactcgtacccgggcaacggtttcacttgagtgcagtggctagtgctcttactcgtacagtgtgcaatactgcgtatcatagtctttgatgtatatcgtattcattcatgttagttgcgtacgttgattgaggtggagccagatgggctattgtttcatatatagactggcagccacctctttggcccagcatgtttgtatacctggaagggaaaactaaagaagctggctagtttagtttgattattatagtagatgtcctaatcactagagattagaatgtcttggcgatgattagtcgtcgtcccctgtatcatgtctagaccaactgtgtcatgaagttggtgctggtgttttacctgtgtactacaagtaggtgtcctagatctagtgtacagagccgtttagacccatgtggacttcaccattaacgatggaaaatgttcattatatgacagtatattacaatggacttgctccatttcttccttgcatcacatgttctccacctccatagttgatcaacacatcatagtagctaaggctgctgctctcccactacagtccaccacaagttaagtagcaccgtcagtacagctaaaagtacacgtctagtacgtttcataactagtcaagtagcccctattacagatatcagcactatcacgcacgagtttttctctgtgctatctaatcaacttgccaagtattcggagaagatacactttcttggcatcaggtatacgagggagcctatcagatgaaaaagggtatattggatccattcatatccacctacacgttgtcataatctcctcattcacgtgattcatttcgtgacactagtttctcactttcccccccgcacctatagtcaacttggcggacacgctacttgtagctgacgttgatttatagacccaatcaaagcgggttatcggtcaggtagcacttatcattcatcgttcatactacgatgagcaatctcgggcatgtccggaaaagtgtcgggcgcgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggcccgacgtcgcatgcattccgacagcagcgactgggcaccatgatcaagcgaaacaccttcccccagctgccctggcaaaccatcaagaaccctactttcatcaagtgcaagaacggttctactcttctcacctccggtgtctacggctggtgccgaaagcctaactacaccgctgatttcatcatgtgcctcacctgggctctcatgtgcggtgttgcttctcccctgccttacttctacccggtcttcttcttcctggtgctcatccaccgagcttaccgagactttgagcgactggagcgaaagtacggtgaggactaccaggagttcaagcgacaggtcccttggatcttcatcccttatgttttctaaacgataagcttagtgagcgaatggtgaggttacttaattgagtggccagcctatgggattgtataacagacagtcaatatattactgaaaagactgaacagccagacggagtgaggttgtgagtgaatcgtagagggcggctattacagcaagtctactctacagtgtactaacacagcagagaacaaatacaggtgtgcattcggctatctgagaattagttggagagctcgagaccctcggcgataaactgctcctcggttttgtgtccatacttgtacggaccattgtaatggggcaagtcgttgagttctcgtcgtccgacgttcagagcacagaaaccaatgtaatcaatgtagcagagatggttctgcaaaagattgatttgtgcgagcaggttaattaagttgcgacacatgtcttgatagtatcttgaattctctctcttgagcttttccataacaagttcttctgcctccaggaagtccatgggtggtttgatcatggttttggtgtagtggtagtgcagtggtggtattgtgactggggatgtagttgagaataagtcatacacaagtcagctttcttcgagcctcatataagtataagtagttcaacgtattagcactgtacccagcatctccgtatcgagaaacacaacaacatgccccattggacagatcatgcggatacacaggttgtgcagtatcatacatactcgatcagacaggtcgtctgaccatcatacaagctgaacaagcgctccatacttgcacgctctctatatacacagttaaattacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggactaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtgacaccggtactggtgcttgacagtgttgccaatatctgcgaactttctgtcctcgaacaggaagaaaccgtgcttaagagcaagttccttgagggggagcacagtgccggcgtaggtgaagtcgtcaatgatgtcgatatgggttttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcgtcgcctttgccgacaaaaatgtgatcatgatgaaagccagcaatgacgttgcagctgatattgttgtcggccaaccgcgccgaaaacgcagctgtcagacccacagcctccaacgaagaatgtatcgtcaaagtgatccaagcacactcatagttggagtcgtactccaaaggcggcaatgacgagtcagacagatactcgtcgaccttttccttgggaaccaccaccgtcagcccttctgactcacgtattgtagccaccgacacaggcaacagtccgtggatagcagaatatgtcttgtcggtccatttctcaccaactttaggcgtcaagtgaatgttgcagaagaagtatgtgccttcattgagaatcggtgttgctgatttcaataaagtcttgagatcagtttggccagtcatgttgtggggggtaattggattgagttatcgcctacagtctgtacaggtatactcgctgcccactttatactttttgattccgctgcacttgaagcaatgtcgtttaccaaaagtgagaatgctccacagaacacaccccagggtatggttgagcaaaaaataaacactccgatacggggaatcgaaccccggtctccacggttctcaagaagtattcttgatgagagcgtatcgatgagcctaaaatgaacccgagtatatctcataaaattctcggtgagaggtctgtgactgtcagtacaaggtgccttcattatgccctcaaccttaccatacctcactgaatgtagtgtacctctaaaaatgaaatacagtgccaaaagccaaggcactgagctcgtctaacggacttgatatacaaccaattaaaacaaatgaaaagaaatacagttctttgtatcatttgtaacaattaccctgtacaaactaaggtattgaaatcccacaatattcccaaagtccacccctttccaaattgtcatgcctacaactcatataccaagcactaacctaccgtttaaaccatcatctaagggcctcaaaactacctcggaactgctgcgctgatctggacaccacagaggttccgagcactttaggttgcaccaaatgtcccaccaggtgcaggcagaaaacgctggaacagcgtgtacagtttgtcttaacaaaaagtgagggcgctgaggtcgagcagggtggtgtgacttgttatagcctttagagctgcgaaagcgcgtatggatttggctcatcaggccagattgagggtctgtggacacatgtcatgttagtgtacttcaatcgccccctggatatagccccgacaataggccgtggcctcatttttttgccttccgcacatttccattgctcggtacccacaccttgcttctcctgcacttgccaaccttaatactggtttacattgaccaacatcttacaagcggggggcttgtctagggtatatataaacagtggctctcccaatcggttgccagtctcttttttcctttctttccccacagattcgaaatctaaactacacatcacaccatggctcccaaggtcatctctaagaacgaatcgcaactggtcgctgaggctgctgccgctgagatcattcgactccagaacgagtcaattgctgccactggagctttccatgttgccgtatctggaggctctctggtgtctgctctccgaaagggtctggtcaacaactcggagaccaagttccccaagtggaagattttcttctccgacgaacggctggtcaagctggacgatgccgactccaactacggtctcctcaagaaggatctgctcgatcacatccccaaggatcagcaaccacaggtcttcaccgtcaaggagtctcttctgaacgactctgatgccgtctccaaggactaccaggagcagattgtcaagaatgtgcctctcaacggccagggagtgcctgttttcgatctcattctgctcggatgcggtcctgatggccacacttgctcgctgttccctggacacgctctgctcaaggaggagaccaagtttgtcgccaccattgaggactctcccaagcctcctcctcgacgaatcaccatcactttccccgttctcaaggctgccaaggccatcgctttcgtcgccgagggagccggaaaggcccctgtcctcaagcagatcttcgaggagcccgagcccactcttccctctgccattgtcaacaaggtcgctaccggacccgttttctggtttgtttccgactctgccgttgagggcgtcaacctctccaagatctagcggccgcatgagaagataaatatataaatacattgagatattaaatgcgctagattagagagcctcatactgctcggagagaagccaagacgagtactcaaaggggattacaccatccatatccacagacacaagctggggaaaggttctatatacactttccggaataccgtagtttccgatgttatcaatgggggcagccaggatttcaggcacttcggtgtctcggggtgaaatggcgttcttggcctccatcaagtcgtaccatgtcttcatttgcctgtcaaagtaaaacagaagcagatgaagaatgaacttgaagtgaaggaattt

SEQ ID NO: 16 - DNA, 37 bp, Artificial Sequence
  Primer YL961
tttccatggctcccaaggtcatctctaagaacgaatc

SEQ ID NO: 17 - DNA, 39 bp, Artificial Sequence
  Primer YL962
tttgcggccgcttagatcttggagaggttgacgccctca

SEQ ID NO: 18 - DNA, 1001 bp, Yarrowia lipolytica
  misc_feature: Promoter FBA
taaacagtgtacgcagtactatagaggaacaattgccccggagaagacggccaggccgcctagatgacaaattcaacaactcacagctgactttctgccattgccactagggggggcctttttatatggccaagccaagctctccacgtcggttgggctgcacccaacaataaatgggtagggttgcaccaacaaagggatgggatggggggtagaagatacgaggataacggggctcaatggcacaaataagaacgaatactgccattaagactcgtgatccagcgactgacaccattgcatcatctaagggcctcaaaactacctcggaactgctgcgctgatctggacaccacagaggttccgagcactttaggttgcaccaaatgtcccaccaggtgcaggcagaaaacgctggaacagcgtgtacagtttgtcttaacaaaaagtgagggcgctgaggtcgagcagggtggtgtgacttgttatagcctttagagctgcgaaagcgcgtatggatttggctcatcaggccagattgagggtctgtggacacatgtcatgttagtgtacttcaatcgccccctggatatagccccgacaataggccgtggcctcatttttttgccttccgcacatttccattgctcggtacccacaccttgcttctcctgcacttgccaaccttaatactggtttacattgaccaacatcttacaagcggggggcttgtctagggtatatataaacagtggctctcccaatcggttgccagtctcttttttcctttctttccccacagattcgaaatctaaactacacatcacacaatgcctgttactgacgtccttaagcgaaagtccggtgtcatcgtcggcgacgatgtccgagccgtgagtatccacgacaagatcagtgtcgagacgacgcgttttgtgtaatgacacaatccgaaagtcgctagcaacacacactctctacacaaactaacccagctctcc

SEQ ID NO: 19 - DNA, 8585 bp, Artificial Sequence
  Plasmid pZKLY-6PGL
ggccgcatgagaagataaatatataaatacattgagatattaaatgcgctagattagagagcctcatactgctcggagagaagccaagacgagtactcaaaggggattacaccatccatatccacagacacaagctggggaaaggttctatatacactttccggaataccgtagtttccgatgttatcaatgggggcagccaggatttcaggcacttcggtgtctcggggtgaaatggcgttcttggcctccatcaagtcgtaccatgtcttcatttgcctgtcaaagtaaaacagaagcagatgaagaatgaacttgaagtgaaggaatttaaatgtaacgaaactgaaatttgaccagatattgtgtccgcggtggagctccagcttttgttccctttagtgagggttaatttcgagcttggcgtaatcatggtcatagctgtttcctgtgtgaaattgttatccgctcacaagcttccacacaacgtacgttgattgaggtggagccagatgggctattgtttcatatatagactggcagccacctctttggcccagcatgtttgtatacctggaagggaaaactaaagaagctggctagtttagtttgattattatagtagatgtcctaatcactagagattagaatgtcttggcgatgattagtcgtcgtcccctgtatcatgtctagaccaactgtgtcatgaagttggtgctggtgttttacctgtgtactacaagtaggtgtcctagatctagtgtacagagccgtttagacccatgtggacttcaccattaacgatggaaaatgttcattatatgacagtatattacaatggacttgctccatttcttccttgcatcacatgttctccacctccatagttgatcaacacatcatagtagctaaggctgctgctctcccactacagtccaccacaagttaagtagcaccgtcagtacagctaaaagtacacgtctagtacgtttcataactagtcaagtagcccctattacagatatcagcactatcacgcacgagtttttctctgtgctatctaatcaacttgccaagtattcggagaagatacactttcttggcatcaggtatacgagggagcctatcagatgaaaaagggtatattggatccattcatatccacctacacgttgtcataatctcctcattcacgtgattcatttcgtgacactagtttctcactttcccccccgcacctatagtcaacttggcggacacgctacttgtagctgacgttgatttatagacccaatcaaagcgggttatcggtcaggtagcacttatcattcatcgttcatactacgatgagcaatctcgggcatgtccggaaaagtgtcgggcgcgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggcccgacgtcgcatgcattccgacagcagcgactgggcaccatgatcaagcgaaacaccttcccccagctgccctggcaaaccatcaagaaccctactttcatcaagtgcaagaacggttctactcttctcacctccggtgtctacggctggtgccgaaagcctaactacaccgctgatttcatcatgtgcctcacctgggctctcatgtgcggtgttgcttctcccctgccttacttctacccggtcttcttcttcctggtgctcatccaccgagcttaccgagactttgagcgactggagcgaaagtacggtgaggactaccaggagttcaagcgacaggtcccttggatcttcatcccttatgttttctaaacgataagcttagtgagcgaatggtgaggttacttaattgagtggccagcctatgggattgtataacagacagtcaatatattactgaaaagactgaacagccagacggagtgaggttgtgagtgaatcgtagagggcggctattacagcaagtctactctacagtgtactaacacagcagagaacaaatacaggtgtgcattcggctatctgagaattagttggagagctcgagaccctcggcgataaactgctcctcggttttgtgtccatacttgtacggaccattgtaatggggcaagtcgttgagttctcgtcgtccgacgttcagagcacagaaaccaatgtaatcaatgtagcagagatggttctgcaaaagattgatttgtgcgagcaggttaattaagttgcgacacatgtcttgatagtatcttgaattctctctcttgagcttttccataacaagttcttctgcctccaggaagtccatgggtggtttgatcatggttttggtgtagtggtagtgcagtggtggtattgtgactggggatgtagttgagaataagtcatacacaagtcagctttcttcgagcctcatataagtataagtagttcaacgtattagcactgtacccagcatctccgtatcgagaaacacaacaacatgccccattggacagatcatgcggatacacaggttgtgcagtatcatacatactcgatcagacaggtcgtctgaccatcatacaagctgaacaagcgctccatacttgcacgctctctatatacacagttaaattacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggactaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtgacaccggtactggtgcttgacagtgttgccaatatctgcgaactttctgtcctcgaacaggaagaaaccgtgcttaagagcaagttccttgagggggagcacagtgccggcgtaggtgaagtcgtcaatgatgtcgatatgggttttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcgtcgcctttgccgacaaaaatgtgatcatgatgaaagccagcaatgacgttgcagctgatattgttgtcggccaaccgcgccgaaaacgcagctgtcagacccacagcctccaacgaagaatgtatcgtcaaagtgatccaagcacactcatagttggagtcgtactccaaaggcggcaatgacgagtcagacagatactcgtcgaccttttccttgggaaccaccaccgtcagcccttctgactcacgtattgtagccaccgacacaggcaacagtccgtggatagcagaatatgtcttgtcggtccatttctcaccaactttaggcgtcaagtgaatgttgcagaagaagtatgtgccttcattgagaatcggtgttgctgatttcaataaagtcttgagatcagtttggccagtcatgttgtggggggtaattggattgagttatcgcctacagtctgtacaggtatactcgctgcccactttatactttttgattccgctgcacttgaagcaatgtcgtttaccaaaagtgagaatgctccacagaacacaccccagggtatggttgagcaaaaaataaacactccgatacggggaatcgaaccccggtctccacggttctcaagaagtattcttgatgagagcgtatcgatgagcctaaaatgaacccgagtatatctcataaaattctcggtgagaggtctgtgactgtcagtacaaggtgccttcattatgccctcaaccttaccatacctcactgaatgtagtgtacctctaaaaatgaaatacagtgccaaaagccaaggcactgagctcgtctaacggacttgatatacaaccaattaaaacaaatgaaaagaaatacagttctttgtatcatttgtaacaattaccctgtacaaactaaggtattgaaatcccacaatattcccaaagtccacccctttccaaattgtcatgcctacaactcatataccaagcactaacctaccgtttaaaccatcatctaagggcctcaaaactacctcggaactgctgcgctgatctggacaccacagaggttccgagcactttaggttgcaccaaatgtcccaccaggtgcaggcagaaaacgctggaacagcgtgtacagtttgtcttaacaaaaagtgagggcgctgaggtcgagcagggtggtgtgacttgttatagcctttagagctgcgaaagcgcgtatggatttggctcatcaggccagattgagggtctgtggacacatgtcatgttagtgtacttcaatcgccccctggatatagccccgacaataggccgtggcctcatttttttgccttccgcacatttccattgctcggtacccacaccttgcttctcctgcacttgccaaccttaatactggtttacattgaccaacatcttacaagcggggggcttgtctagggtatatataaacagtggctctcccaatcggttgccagtctcttttttcctttctttccccacagattcgaaatctaaactacacatcacaccatggctcccaaggtcatctctaagaacgaatcgcaactggtcgctgaggctgctgccgctgagatcattcgactccagaacgagtcaattgctgccactggagctttccatgttgccgtatctggaggctctctggtgtctgctctccgaaagggtctggtcaacaactcggagaccaagttccccaagtggaagattttcttctccgacgaacggctggtcaagctggacgatgccgactccaactacggtctcctcaagaaggatctgctcgatcacatccccaaggatcagcaaccacaggtcttcaccgtcaaggagtctcttctgaacgactctgatgccgtctccaaggactaccaggagcagattgtcaagaatgtgcctctcaacggccagggagtgcctgttttcgatctcattctgctcggatgcggtcctgatggccacacttgctcgctgttccctggacacgctctgctcaaggaggagaccaagtttgtcgccaccattgaggactctcccaagcctcctcctcgacgaatcaccatcactttccccgttctcaaggctgccaaggccatcgctttcgtcgccgagggagccggaaaggcccctgtcctcaagcagatcttcgaggagcccgagcccactcttccctctgccattgtcaacaaggtcgctaccggacccgttttctggtttgtttccgactctgccgttgagggcgtcaacctctccaagatctagc

SEQ ID NO: 20 - DNA, 35 bp, Artificial Sequence
  Primer YL959
tttccatggctggcaccttacccaagttcggcgac

SEQ ID NO: 21 - DNA, 39 bp, Artificial Sequence
  Primer YL960
tttgcggccgctcacgaggagcccttggtgacaggccat

SEQ ID NO: 22 - DNA, 9519 bp, Artificial Sequence
  Plasmid pDMW224-S2
catggatggtacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccgggatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttacgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaagtgcacgggaatatttcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgattaattaactagagcggccgccaccgcggcccgagattccggcctcttcggccgccaagcgacccgggtggacgtctagaggtacctagcaattaacagatagtttgccggtgataattctcttaacctcccacactcctttgacataacgatttatgtaacgaaactgaaatttgaccagatattgtgtccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccgggccccccctcgaggtcgatggtgtcgataagcttgatatcgaattcatgtcacacaaaccgatcttcgcctcaaggaaacctaattctacatccgagagactgccgagatccagtctacactgattaattttcgggccaataatttaaaaaaatcgtgttatataatattatatgtattatatatatacatcatgatgatactgacagtcatgtcccattgctaaatagacagactccatctgccgcctccaactgatgttctcaatatttaaggggtcatctcgcattgtttaataataaacagactccatctaccgcctccaaatgatgttctcaaaatatattgtatgaacttatttttattacttagtattattagacaacttacttgctttatgaaaaacacttcctatttaggaaacaatttataatggcagttcgttcatttaacaatttatgtagaataaatgttataaatgcgtatgggaaatcttaaatatggatagcataaatgatatctgcattgcctaattcgaaatcaacagcaacgaaaaaaatcccttgtacaacataaatagtcatcgagaaatatcaactatcaaagaacagctattcacacgttactattgagattattattggacgagaatcacacactcaactgtctttctctcttctagaaatacaggtacaagtatgtactattctcattgttcatacttctagtcatttcatcccacatattccttggatttctctccaatgaatgacattctatcttgcaaattcaacaattataataagatataccaaagtagcggtatagtggcaatcaaaaagcttctctggtgtgcttctcgtatttatttttattctaatgatccattaaaggtatatatttatttcttgttatataatccttttgtttattacatgggctggatacataaaggtattttgatttaattttttgcttaaattcaatcccccctcgttcagtgtcaactgtaatggtaggaaattaccatacttttgaagaagcaaaaaaaatgaaagaaaaaaaaaatcgtatttccaggttagacgttccgcagaatctagaatgcggtatgcggtacattgttcttcgaacgtaaaagttgcgctccctgagatattgtacatttttgcttttacaagtacaagtacatcgtacaactatgtactactgttgatgcatccacaacagtttgttttgtttttttttgttttttttttttctaatgattcattaccgctatgtatacctacttgtacttgtagtaagccgggttattggcgttcaattaatcatagacttatgaatctgcacggtgtgcgctgcgagttacttttagcttatgcatgctacttgggtgtaatattgggatctgttcggaaatcaacggatgctcaaccgatttcgacagtaataatttgaatcgaatcggagcctaaaatgaacccgagtatatctcataaaattctcggtgagaggtctgtgactgtcagtacaaggtgccttcattatgccctcaaccttaccatacctcactgaatgtagtgtacctctaaaaatgaaatacagtgccaaaagccaaggcactgagctcgtctaacggacttgatatacaaccaattaaaacaaatgaaaagaaatacagttctttgtatcatttgtaacaattaccctgtacaaactaaggtattgaaatcccacaatattcccaaagtccacccctttccaaattgtcatgcctacaactcatataccaagcactaacctaccaaacaccactaaaaccccacaaaatatatcttaccgaatatacagtaacaagctaccaccacactcgttgggtgcagtcgccagcttaaagatatctatccacatcagccacaactcccttcctttaataaaccgactacacccttggctattgaggttatgagtgaatatactgtagacaagacactttcaagaagactgtttccaaaacgtaccactgtcctccactacaaacacacccaatctgcttcttctagtcaaggttgctacaccggtaaattataaatcatcatttcattagcagggcagggccctttttatagagtcttatacactagcggaccctgccggtagaccaacccgcaggcgcgtcagtttgctccttccatcaatgcgtcgtagaaacgacttactccttcttgagcagctccttgaccttgttggcaacaagtctccgacctcggaggtggaggaagagcctccgatatcggcggtagtgataccagcctcgacggactccttgacggcagcctcaacagcgtcaccggcgggcttcatgttaagagagaacttgagcatcatggcggcagacagaatggtggcaatggggttgaccttctgcttgccgagatcgggggcagatccgtgacagggctcgtacagaccgaacgcctcgttggtgtcgggcagagaagccagagaggcggagggcagcagacccagagaaccggggatgacggaggcctcgtcggagatgatatcgccaaacatgttggtggtgatgatgataccattcatcttggagggctgcttgatgaggatcatggcggccgagtcgatcagctggtggttgagctcgagctgggggaattcgtccttgaggactcgagtgacagtctttcgccaaagtcgagaggaggccagcacgttggccttgtcaagagaccacacgggaagaggggggttgtgctgaagggccaggaaggcggccattcgggcaattcgctcaacctcaggaacggagtaggtctcggtgtcggaagcgacgccagatccgtcatcctcctttcgctctccaaagtagatacctccgacgagctctcggacaatgatgaagtcggtgccctcaacgtttcggatgggggagagatcggcgagcttgggcgacagcagctggcagggtcgcaggttggcgtacaggttcaggtcctttcgcagcttgaggagaccctgctcgggtcgcacgtcggttcgtccgtcgggagtggtccatacggtgttggcagcgcctccgacagcaccgagcataatagagtcagcctttcggcagatgtcgagagtagcgtcggtgatgggctcgccctccttctcaatggcagctcctccaatgagtcggtcctcaaacacaaactcggtgccggaggcctcagcaacagacttgagcaccttgacggcctcggcaatcacctcggggccacagaagtcgccgccgagaagaacaatcttcttggagtcagtcttggtcttcttagtttcgggttccattgtggatgtgtgtggttgtatgtgtgatgtggtgtgtggagtgaaaatctgtggctggcaaacgctcttgtatatatacgcacttttgcccgtgctatgtggaagactaaacctccgaagattgtgactcaggtagtgcggtatcggctagggacccaaaccttgtcgatgccgatagcgctatcgaacgtaccccagccggccgggagtatgtcggaggggacatacgagatcgtcaagggtttgtggccaactggtaaataaatgatgtcgaccattaattctcacgtgacacagattattaacgtctcgtaccaaccacagattacgacccattcgcagtcacagttcactagggtttgggttgcatccgttgagagcggtttgtttttaaccttctccatgtgctcactcaggttttgggttcagatcaaatcaaggcgtgaaccactttgtttgaggacaaatgtgacacaaccaaccagtgtcaggggcaagtccgtgacaaaggggaagatacaatgcaattactgacagttacagactgcctcgatgccctaaccttgccccaaaataagacaactgtcctcgtttaagcgcaaccctattcagcgtcacgtcatttaaatgcgtttggatagcactagtctatgaggagcgttttatgttgcggtgagggcgattggtgctcatatgggttcaattgaggtggcggaacgagcttagtcttcaattgaggtgcgagcgacacaattgggtgtcacgtggcctaattgacctcgggtcgtggagtccccagttatacagcaaccacgaggtgcatgggtaggagacgtcaccagacaatagggttttttttggactggagagggttgggcaaaagcgctcaacgggctgtttggggagctgtgggggaggaattggcgatatttgtgaggttaacggctccgatttgcgtgttttgtcgctcctgcatctccccatacccatatcttccctccccacctctttccacgataattttacggatcagcaataaggttccttctcctagtttccacgtccatatatatctatgctgcgtcgtccttttcgtgacatcaccaaaacacatacaaccatggctgttactgacgtccttaagcgaaagtccggtgtcatcgtcggcgacgatgtccgagccgtgagtatccacgacaagatcagtgtcgagacgacgcgttttgtgtaatgacacaatccgaaagtcgctagcaacacacactctctacacaaactaacccagctctc

SEQ ID NO: 23 - DNA, 8500 bp, Artificial Sequence
  Plasmid pGPM-G6PD
ggccgcaagtgtggatggggaagtgagtgcccggttctgtgtgcacaattggcaatccaagatggatggattcaacacagggatatagcgagctacgtggtggtgcgaggatatagcaacggatatttatgtttgacacttgagaatgtacgatacaagcactgtccaagtacaatactaaacatactgtacatactcatactcgtacccgggcaacggtttcacttgagtgcagtggctagtgctcttactcgtacagtgtgcaatactgcgtatcatagtctttgatgtatatcgtattcattcatgttagttgcgtacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccgggccccccctcgaggtcgatggtgtcgataagcttgatatcgaattcatgtcacacaaaccgatcttcgcctcaaggaaacctaattctacatccgagagactgccgagatccagtctacactgattaattttcgggccaataatttaaaaaaatcgtgttatataatattatatgtattatatatatacatcatgatgatactgacagtcatgtcccattgctaaatagacagactccatctgccgcctccaactgatgttctcaatatttaaggggtcatctcgcattgtttaataataaacagactccatctaccgcctccaaatgatgttctcaaaatatattgtatgaacttatttttattacttagtattattagacaacttacttgctttatgaaaaacacttcctatttaggaaacaatttataatggcagttcgttcatttaacaatttatgtagaataaatgttataaatgcgtatgggaaatcttaaatatggatagcataaatgatatctgcattgcctaattcgaaatcaacagcaacgaaaaaaatcccttgtacaacataaatagtcatcgagaaatatcaactatcaaagaacagctattcacacgttactattgagattattattggacgagaatcacacactcaactgtctttctctcttctagaaatacaggtacaagtatgtactattctcattgttcatacttctagtcatttcatcccacatattccttggatttctctccaatgaatgacattctatcttgcaaattcaacaattataataagatataccaaagtagcggtatagtggcaatcaaaaagcttctctggtgtgcttctcgtatttatttttattctaatgatccattaaaggtatatatttatttcttgttatataatccttttgtttattacatgggctggatacataaaggtattttgatttaattttttgcttaaattcaatcccccctcgttcagtgtcaactgtaatggtaggaaattaccatacttttgaagaagcaaaaaaaatgaaagaaaaaaaaaatcgtatttccaggttagacgttccgcagaatctagaatgcggtatgcggtacattgttcttcgaacgtaaaagttgcgctccctgagatattgtacatttttgcttttacaagtacaagtacatcgtacaactatgtactactgttgatgcatccacaacagtttgttttgtttttttttgttttttttttttctaatgattcattaccgctatgtatacctacttgtacttgtagtaagccgggttattggcgttcaattaatcatagacttatgaatctgcacggtgtgcgctgcgagttacttttagcttatgcatgctacttgggtgtaatattgggatctgttcggaaatcaacggatgctcaatcgatttcgacagtaattaattaagtcatacacaagtcagctttcttcgagcctcatataagtataagtagttcaacgtattagcactgtacccagcatctccgtatcgagaaacacaacaacatgccccattggacagatcatgcggatacacaggttgtgcagtatcatacatactcgatcagacaggtcgtctgaccatcatacaagctgaacaagcgctccatacttgcacgctctctatatacacagttaaattacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggactaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtgacaccggtactggtgcttgacagtgttgccaatatctgcgaactttctgtcctcgaacaggaagaaaccgtgcttaagagcaagttccttgagggggagcacagtgccggcgtaggtgaagtcgtcaatgatgtcgatatgggttttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcgtcgcctttgccgacaaaaatgtgatcatgatgaaagccagcaatgacgttgcagctgatattgttgtcggccaaccgcgccgaaaacgcagctgtcagacccacagcctccaacgaagaatgtatcgtcaaagtgatccaagcacactcatagttggagtcgtactccaaaggcggcaatgacgagtcagacagatactcgtcgacgtttaaacagtgtacgcagatctactatagaggaacatttaaatgcgtttggatagcactagtctatgaggagcgttttatgttgcggtgagggcgattggtgctcatatgggttcaattgaggtggcggaacgagcttagtcttcaattgaggtgcgagcgacacaattgggtgtcacgtggcctaattgacctcgggtcgtggagtccccagttatacagcaaccacgaggtgcatgggtaggagacgtcaccagacaatagggttttttttggactggagagggttgggcaaaagcgctcaacgggctgtttggggagctgtgggggaggaattggcgatatttgtgaggttaacggctccgatttgcgtgttttgtcgctcctgcatctccccatacccatatcttccctccccacctctttccacgataattttacggatcagcaataaggttccttctcctagtttccacgtccatatatatctatgctgcgtcgtccttttcgtgacatcaccaaaacacatacaaccatggctggcaccttacccaagttcggcgacggaaccaccattgtggttcttggagcctccggcgacctcgctaagaagaagaccgtgagtattgaaccagactgaggtcaattgaagagtaggagagtctgagaacattcgacggacctgattgtgctctggaccactcaattgactcgttgagagccccaatgggtcttggctagccgagtcgttgacttgttgacttgttgagcccagaacccccaacttttgccaccatacaccgccatcaccatgacacccagatgtgcgtgcgtatgtgagagtcaattgttccgtggcaaggcacagcttattccaccgtgttccttgcacaggtggtctttacgctctcccactctatccgagcaataaaagcggaaaaacagcagcaagtcccaacagacttctgctccgaataaggcgtctagcaagtgtgcccaaaactcaattcaaaaatgtcagaaacctgatatcaacccgtcttcaaaagctaaccccagttccccgccctcttcggcctttaccgaaacggcctgctgcccaaaaatgttgaaatcatcggctacgcacggtcgaaaatgactcaggaggagtaccacgagcgaatcagccactacttcaagacccccgacgaccagtccaaggagcaggccaagaagttccttgagaacacctgctacgtccagggcccttacgacggtgccgagggctaccagcgactgaatgaaaagattgaggagtttgagaagaagaagcccgagccccactaccgtcttttctacctggctctgccccccagcgtcttccttgaggctgccaacggtctgaagaagtatgtctaccccggcgagggcaaggcccgaatcatcatcgagaagccctttggccacgacctggcctcgtcacgagagctccaggacggccttgctcctctctggaaggagtctgagatcttccgaatcgaccactacctcggaaaggagatggtcaagaacctcaacattctgcgatttggcaaccagttcctgtccgccgtgtgggacaagaacaccatttccaacgtccagatctccttcaaggagccctttggcactgagggccgaggtggatacttcaacgacattggaatcatccgagacgttattcagaaccatctgttgcaggttctgtccattctagccatggagcgacccgtcactttcggcgccgaggacattcgagatgagaaggtcaaggtgctccgatgtgtcgacattctcaacattgacgacgtcattctcggccagtacggcccctctgaagacggaaagaagcccggatacaccgatgacgatggcgttcccgatgactcccgagctgtgacctttgctgctctccatctccagatccacaacgacagatgggagggtgttcctttcatcctccgagccggtaaggctctggacgagggcaaggtcgagatccgagtgcagttccgagacgtgaccaagggcgttgtggaccatctgcctcgaaatgagctcgtcatccgaatccagccctccgagtccatctacatgaagatgaactccaagctgcctggccttactgccaagaacattgtcaccgacctggatctgacctacaaccgacgatactcggacgtgcgaatccctgaggcttacgagtctctcattctggactgcctcaagggtgaccacaccaactttgtgcgaaacgacgagctggacatttcctggaagattttcaccgatctgctgcacaagattgacgaggacaagagcattgtgcccgagaagtacgcctacggctctcgtggccccgagcgactcaagcagtggctccgagaccgaggctacgtgcgaaacggcaccgagctgtaccaatggcctgtcaccaagggctcctcgtgagc

SEQ ID NO: 24 - DNA, 878 bp, Yarrowia lipolytica
  misc_feature: Promoter GPM
gcctctgaatactttcaacaagttacacccttcattaattctcacgtgacacagattattaacgtctcgtaccaaccacagattacgacccattcgcagtcacagttcactagggtttgggttgcatccgttgagagcggtttgtttttaaccttctccatgtgctcactcaggttttgggttcagatcaaatcaaggcgtgaaccactttgtttgaggacaaatgtgacacaaccaaccagtgtcaggggcaagtccgtgacaaaggggaagatacaatgcaattactgacagttacagactgcctcgatgccctaaccttgccccaaaataagacaactgtcctcgtttaagcgcaaccctattcagcgtcacgtcataatagcgtttggatagcactagtctatgaggagcgttttatgttgcggtgagggcgattggtgctcatatgggttcaattgaggtggcggaacgagcttagtcttcaattgaggtgcgagcgacacaattgggtgtcacgtggcctaattgacctcgggtcgtggagtccccagttatacagcaaccacgaggtgcatgggtaggagacgtcaccagacaatagggttttttttggactggagagggttgggcaaaagcgctcaacgggctgtttggggagctgtgggggaggaattggcgatatttgtgaggttaacggctccgatttgcgtgttttgtcgctcctgcatctccccatacccatatcttccctccccacctctttccacgataattttacggatcagcaataaggttccttctcctagtttccacgtccatatatatctatgctgcgtcgtccttttcgtgacatcaccaaaacacatacaaaaatg

SEQ ID NO: 25 - DNA, 9045 bp, Artificial Sequence
  Plasmid pZKLY
catggccacccgacagcgaactgctaccactgtcgtggtcgaggacctgcccaaggttaccctcgaggccaagtccgaacctgtctttcccgacatcaagaccatcaaggatgccattcctgctcactgctttcagccctctctggtcacctccttctactatgtgttccgagactttgctatggtttctgccctcgtctgggctgcccttacctacattccctcgatccctgatcagactctgcgagtggcagcttggatggtctacggcttcgttcagggactcttctgtaccggtgtctggattctcggacacgagtgcggtcatggagccttctctctgcacggcaaggtcaacaatgtcaccggatggtttcttcattccttcctgctcgttccctacttcagctggaagtactctcatcaccgacatcaccgattcacaggtcacatggatctggacatggctttcgttcccaagaccgagcccaaaccctccaagtctctcatgattgctggcattgacgttgccgaacttgtcgaggacactcctgctgcccagatggtcaagctcatcttccatcagctgttcggatggcaggcgtacctcttcttcaacgccagctctggcaagggttccaagcagtgggagcccaagactggactctcgaagtggtttcgagtgtctcacttcgagcctaccagcgctgtcttcagacccaacgaggccatcttcattctcatctcggacatcggtcttgctctcatgggcactgcactgtactttgcttccaagcaagtcggagtttctaccattctgttcctctaccttgttccctacctgtgggtccatcactggctcgtggccattacttaccttcaccatcaccataccgaactgcctcactacaccgctgagggctggacctacgtcaagggtgcactcgccactgtggatcgagagtttggattcatcggcaagcatctctttcacggtatcattgagaagcacgttgtgcatcacttgtttcccaagattcccttctacaaggctgacgaagccaccgaggccatcaagcctgtcattggcgaccactactgtcacgacgatcggtccttcctgggtcagctgtggaccatcttcggaactctcaagtacgtggagcacgatcctgcccgacccggtgccatgcgatggaacaaggactaagcggccgcatgagaagataaatatataaatacattgagatattaaatgcgctagattagagagcctcatactgctcggagagaagccaagacgagtactcaaaggggattacaccatccatatccacagacacaagctggggaaaggttctatatacactttccggaataccgtagtttccgatgttatcaatgggggcagccaggatttcaggcacttcggtgtctcggggtgaaatggcgttcttggcctccatcaagtcgtaccatgtcttcatttgcctgtcaaagtaaaacagaagcagatgaagaatgaacttgaagtgaaggaatttaaatgtaacgaaactgaaatttgaccagatattgtgtccgcggtggagctccagcttttgttccctttagtgagggttaatttcgagcttggcgtaatcatggtcatagctgtttcctgtgtgaaattgttatccgctcacaagcttccacacaacgtacgttgattgaggtggagccagatgggctattgtttcatatatagactggcagccacctctttggcccagcatgtttgtatacctggaagggaaaactaaagaagctggctagtttagtttgattattatagtagatgtcctaatcactagagattagaatgtcttggcgatgattagtcgtcgtcccctgtatcatgtctagaccaactgtgtcatgaagttggtgctggtgttttacctgtgtactacaagtaggtgtcctagatctagtgtacagagccgtttagacccatgtggacttcaccattaacgatggaaaatgttcattatatgacagtatattacaatggacttgctccatttcttccttgcatcacatgttctccacctccatagttgatcaacacatcatagtagctaaggctgctgctctcccactacagtccaccacaagttaagtagcaccgtcagtacagctaaaagtacacgtctagtacgtttcataactagtcaagtagcccctattacagatatcagcactatcacgcacgagtttttctctgtgctatctaatcaacttgccaagtattcggagaagatacactttcttggcatcaggtatacgagggagcctatcagatgaaaaagggtatattggatccattcatatccacctacacgttgtcataatctcctcattcacgtgattcatttcgtgacactagtttctcactttcccccccgcacctatagtcaacttggcggacacgctacttgtagctgacgttgatttatagacccaatcaaagcgggttatcggtcaggtagcacttatcattcatcgttcatactacgatgagcaatctcgggcatgtccggaaaagtgtcgggcgcgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggcccgacgtcgcatgcattccgacagcagcgactgggcaccatgatcaagcgaaacaccttcccccagctgccctggcaaaccatcaagaaccctactttcatcaagtgcaagaacggttctactcttctcacctccggtgtctacggctggtgccgaaagcctaactacaccgctgatttcatcatgtgcctcacctgggctctcatgtgcggtgttgcttctcccctgccttacttctacccggtcttcttcttcctggtgctcatccaccgagcttaccgagactttgagcgactggagcgaaagtacggtgaggactaccaggagttcaagcgacaggtcccttggatcttcatcccttatgttttctaaacgataagcttagtgagcgaatggtgaggttacttaattgagtggccagcctatgggattgtataacagacagtcaatatattactgaaaagactgaacagccagacggagtgaggttgtgagtgaatcgtagagggcggctattacagcaagtctactctacagtgtactaacacagcagagaacaaatacaggtgtgcattcggctatctgagaattagttggagagctcgagaccctcggcgataaactgctcctcggttttgtgtccatacttgtacggaccattgtaatggggcaagtcgttgagttctcgtcgtccgacgttcagagcacagaaaccaatgtaatcaatgtagcagagatggttctgcaaaagattgatttgtgcgagcaggttaattaagttgcgacacatgtcttgatagtatcttgaattctctctcttgagcttttccataacaagttcttctgcctccaggaagtccatgggtggtttgatcatggttttggtgtagtggtagtgcagtggtggtattgtgactggggatgtagttgagaataagtcatacacaagtcagctttcttcgagcctcatataagtataagtagttcaacgtattagcactgtacccagcatctccgtatcgagaaacacaacaacatgccccattggacagatcatgcggatacacaggttgtgcagtatcatacatactcgatcagacaggtcgtctgaccatcatacaagctgaacaagcgctccatacttgcacgctctctatatacacagttaaattacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggactaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtgacaccggtactggtgcttgacagtgttgccaatatctgcgaactttctgtcctcgaacaggaagaaaccgtgcttaagagcaagttccttgagggggagcacagtgccggcgtaggtgaagtcgtcaatgatgtcgatatgggttttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcgtcgcctttgccgacaaaaatgtgatcatgatgaaagccagcaatgacgttgcagctgatattgttgtcggccaaccgcgccgaaaacgcagctgtcagacccacagcctccaacgaagaatgtatcgtcaaagtgatccaagcacactcatagttggagtcgtactccaaaggcggcaatgacgagtcagacagatactcgtcgaccttttccttgggaaccaccaccgtcagcccttctgactcacgtattgtagccaccgacacaggcaacagtccgtggatagcagaatatgtcttgtcggtccatttctcaccaactttaggcgtcaagtgaatgttgcagaagaagtatgtgccttcattgagaatcggtgttgctgatttcaataaagtcttgagatcagtttggccagtcatgttgtggggggtaattggattgagttatcgcctacagtctgtacaggtatactcgctgcccactttatactttttgattccgctgcacttgaagcaatgtcgtttaccaaaagtgagaatgctccacagaacacaccccagggtatggttgagcaaaaaataaacactccgatacggggaatcgaaccccggtctccacggttctcaagaagtattcttgatgagagcgtatcgatgagcctaaaatgaacccgagtatatctcataaaattctcggtgagaggtctgtgactgtcagtacaaggtgccttcattatgccctcaaccttaccatacctcactgaatgtagtgtacctctaaaaatgaaatacagtgccaaaagccaaggcactgagctcgtctaacggacttgatatacaaccaattaaaacaaatgaaaagaaatacagttctttgtatcatttgtaacaattaccctgtacaaactaaggtattgaaatcccacaatattcccaaagtccacccctttccaaattgtcatgcctacaactcatataccaagcactaacctaccgtttaaaccatcatctaagggcctcaaaactacctcggaactgctgcgctgatctggacaccacagaggttccgagcactttaggttgcaccaaatgtcccaccaggtgcaggcagaaaacgctggaacagcgtgtacagtttgtcttaacaaaaagtgagggcgctgaggtcgagcagggtggtgtgacttgttatagcctttagagctgcgaaagcgcgtatggatttggctcatcaggccagattgagggtctgtggacacatgtcatgttagtgtacttcaatcgccccctggatatagccccgacaataggccgtggcctcatttttttgccttccgcacatttccattgctcggtacccacaccttgcttctcctgcacttgccaaccttaatactggtttacattgaccaacatcttacaagcggggggcttgtctagggtatatataaacagtggctctcccaatcggttgccagtctcttttttcctttctttccccacagattcgaaatctaaactacacatcacac